(19) United States
(12) Patent Application Publication
(10) Pub. No.: US 2019/0262431 A1
(43) Pub. Date: Aug. 29, 2019

(54) CBL-FAMILY INHIBITOR COMPRISING A CBL-FAMILY TKB DOMAIN BINDING PEPTIDE FOR THE PREVENTION OR TREATMENT OF DISEASES

(71) Applicants: IMBA - INSTITUT FÜR MOLEKULARE BIOTECHNOLOGIE GMBH, Vienna (AT); MEDIZINISCHE UNIVERSITÄT WIEN, Vienna (AT)

(72) Inventors: Josef PENNINGER, Vienna (AT); Gerald WIRNSBERGER, Vienna (AT); Florian ZWOLANEK, Vienna (AT); Karl KUCHLER, Bad Vöslau (AT)

(21) Appl. No.: 16/307,735

(22) PCT Filed: Jun. 9, 2017
(86) PCT No.: PCT/EP2017/064072

§ 371 Date: Dec. 6, 2018

(30) Foreign Application Priority Data
Jun. 9, 2016 (EP) 16173740.8

(51) Int. Cl. A61K 38/45; A61K 47/50; C12N 15/52; A61P 33/00; A61P 35/00; A61P 37/04; A61P 31/10; C12N 9/12; A61K 9/00

(52) U.S. Cl. CPC A61K 38/45; A61K 47/50; C12N 15/52; A61P 33/00; A61K 9/0014; A61P 37/04; A61P 31/10; C12N 9/1205; A61K 9/0019; A61P 35/00

(57) ABSTRACT

The present disclosure relates to the prevention or treatment of diseases by regulating innate immunity. A Cbl-family inhibitor is provided for use in the prevention or treatment of diseases in a patient, a Cbl-family inhibitor including a binding peptide of Syk, a fusion peptide including a cell delivery portion and a Cbl-family TKB domain binding peptide, wherein the Cbl-family TKB domain binding peptide binds to the TKB domain of a Cbl-family protein, kits and uses for the same.

### FIELD OF THE INVENTION

The present invention relates to novel Cbl-family protein inhibitors and the inhibition of a Cbl-family member to modulate the activity of the immune system, especially in the prevention or treatment of fungal infections, in particular candidiasis, or cancer.

### BACKGROUND OF THE INVENTION

The Cbl-family of E3-ubiquitin-ligases contains three protein members, Cbl (also known as c-Cbl), Cbl-b and Cbl-c (also known as Cbl-3). Cbl-b (casitas b-lineage lymphoma-b) has originally been described as a negative regulator of the adaptive immune response. Both T cell receptor (TCR) signaling and B cell receptor (BCR) signaling are modulated by Cbl-b mediated ubiquitination of critical signaling molecules. Consequently Cbl-b has been a target for immune therapy in the treatment of several diseases, especially cancer (US 2010/0260808 A). U.S. Pat. No. 8,168,176 B2 describes methods of treating endotoxin-mediated disorders, including sepsis by administering Cbl-b. Additional roles for Cbl-b are in NK cell-mediated anti-tumor immunity (US 2014/0010781 A), FcεR signaling in mast cells, integrin signaling, and DC functions, extending the range of Cbl-b functions to cells of the innate immune system. Signaling molecules targeted by Cbl-b are often not specific to defined pathways, but can be used by multiple signaling modules.

Kawai, 2015, relates to a prevention of skeletal muscle atrophy by cell delivery of a pentapeptide as Cbl-b inhibitor with a atelocollagen (ATCOL)-based delivery method.

Kumar, 2012 relates to an identification of Cbl(TKB) (also known as c-Cbl) inhibitors.

Ruzza, 2009, relates to therapeutic prospects of Syk inhibitors. Syk inhibitors have the opposite effect of Cbl inhibitors, which enhance Syk's effect.

Fungi are increasingly recognized as major pathogens in critically ill patients (Enoch et al., 2006) and fungal infections claim an estimated 1.5 million lives every year (Brown et al., 2012). Candida species and Cryptococcus species are the yeasts most frequently isolated in clinical practice. Candida albicans is the most frequent fungal pathogen isolated from patients suffering from severe forms of fungal infections in the developed world (Kullberg et al., 2015). Aspergillus species are the most commonly isolated invasive molds (Denning, 1998). The use of antineoplastic and immunosuppressive agents, broad-spectrum antibiotics, prosthetic devices and grafts, and more aggressive surgery are reasons for the increase in invasive fungal infections. Patients with burns or pancreatitis are also predisposed to fungal infection (Enoch et al., 2006). Patients with an impaired immune status, as observed upon HIV infection (Armstrong-James et al., 2014; Brown et al., 2014; Park et al., 2009), immune suppression (Ravikumar et al., 2015), or certain types of primary immune deficiencies (Lanternier et al., 2013; Lortholary et al., 1997) are at high risk of developing a severe fungal infection.

Yeasts, molds and dimorphs are three types of fungi known to cause disease in humans (Jain et al., 2010). Fungal infections are classified into 3 groups: 1) superficial/cutaneous infections, present on skin, hair and nails, 2) subcutaneous infections, present in tissues under the skin and 3) systemic infections.

Several anti-fungal agents are used for the treatment of the different kinds of fungal infections. Amphotericin B, a polyene with a very broad spectrum of activity, including most yeasts and molds, belongs to the class of polyene anti-fungals and can be administered locally or systemically. However, side effects are occurring in 50-90% of cases, and are usually nephrotoxicity or infusion-related. Fever, chills, nausea, vomiting and hypotension are further side effects. Fluconazole belongs to the class of azole anti-fungals and is administered orally or intravenously. Disadvantages include resistance of several Candida strains and adverse effects such as headache, nausea, vomiting and elevated liver enzymes.

Furthermore, the different functions of Cbl proteins, and their roles both in the development of cancer and the regulation of immune responses to cancer provide multiple therapeutic opportunities. Thus, targeting the pathways regulated by Cbl proteins may provide attractive opportunities for treating cancer.

Taken together, efficacy and/or safety of known anti-fungal or anti-cancer agents are still lacking in many circumstances and there is a significant need for new anti-fungal and anti-cancer therapies. It is one of the objects of the present invention to provide novel compounds and methods for preventing or treating fungal infections and/or cancer.

### SUMMARY OF THE INVENTION

The present invention provides methods and means for inhibition of casitas b-lineage lymphoma-family (Cbl-family) activity, which can significantly ameliorate patient's health when diseased or infected, particularly by fungal infections or cancer, most strikingly by significantly reducing morbidity (see e.g. FIG. 1a) and mortality (FIG. 1b) in fungal infections; and by significant reduction of tumor growth in cancer (FIG. 15a). Accordingly, the present invention provides a Cbl-family protein inhibitor for use in the prevention or treatment of a fungal infection and/or cancer in a patient. Preferably, the fungal infection is a yeast infection, more preferably an opportunistic yeast infection. Even more preferably, the fungal infection is a candidiasis or a cryptococcosis. In an embodiment, the Cbl-family protein inhibitor comprises a binding peptide of spleen tyrosine kinase (Syk).

In another aspect of the invention, a fusion peptide comprising a cell delivery portion and a Cbl-family protein binding peptide, wherein the Cbl-family protein binding peptide binds to the tyrosine kinase binding (TKB) domain of a Cbl-family member (hence the peptide is also referred to as Cbl-family TKB domain binding peptide), is provided. Notably, this fusion peptide has turned out to be very effective in protecting from lethal fungal infections (FIG. 12b) and cancer (FIG. 15a), but it can be used as Cbl-family protein inhibitor in any application or use, including for use in therapy.

Moreover, the invention relates to a fusion peptide comprising a cell delivery portion and a Cbl-family TKB domain binding peptide, wherein the Cbl-family TKB domain binding peptide is an intramer or aptamer, and wherein the Cbl-family TKB domain binding peptide inhibits a Cbl-family protein. Preferably, the Cbl-family binding peptide binds to the TKB domain of a Cbl-family member, preferably Cbl-b.

The present invention further relates to a Cbl-family protein inhibitor for use in a patient as a vaccine adjuvant, wherein the Cbl-family protein inhibitor is a Cbl-family TKB domain binding peptide. Preferably, the Cbl-family binding peptide binds to the TKB domain of a Cbl-family protein. Especially preferred is a peptide (fragment) of or derived from Syk.

The present invention also relates to a pharmaceutical composition, comprising the fusion peptide or the Cbl-family family TKB domain binding peptide, and a pharmaceutically acceptable carrier promoting cellular uptake of said peptide.

Even further, the invention provides a vaccine, comprising as an adjuvant a Cbl-family protein inhibitor. Preferably, the Cbl-family protein inhibitor is a Cbl-family TKB domain binding peptide of Syk, and at least one antigen.

In another aspect, the present invention also relates to a kit comprising said peptide and a pharmaceutically acceptable excipient. Preferably, the pharmaceutically acceptable excipient is suitable for intracellular administration in the patient.

The present invention also relates to a method for regulating innate immunity, comprising the inhibition of a Cbl-family protein. Preferably, said fusion peptide or Cbl-family protein inhibitor is administered to the patient.

Moreover, in another aspect, the present invention relates to an in vitro or in vivo method for stimulating or activating an immune cell, comprising contacting or treating the cell with said fusion peptide or Cbl-family TKB domain binding peptide.

All aspects and embodiments of the invention relate to the inhibition of Cbl-family proteins using a Cbl-family protein binding peptide. All general and specific descriptions provided herein in more detail, including preferred embodiments relate to all aspect and uses of the invention equally, except where explicitly stated. E.g. any preferred definition of the peptide or its Cbl-family TKB domain binding portion can be used in any aspect or method of the invention.

### DETAILED DESCRIPTION OF THE INVENTION

The Cbl-family of E3-ubiquitin-ligases plays a key role in regulating signaling in a variety of cellular systems (Sohn et al., 2003). The amino-terminal domain of Cbl proteins is composed of a tyrosine kinase binding (TKB) domain and a RING finger domain, which are both modulating signal transduction and protein degradation (Ohno et al., 2016; Peschard et al., 2004). Substrate recognition by Cbl-family members, such as Cbl-b, is mainly mediated by its TKB domain, which recognizes phosphotyrosines on target molecules, such as ZAP-70, Syk and Src and with residues located in RTKs such as EGFR, Met and CSF-1 receptors (Mancini et al., 2002; Peschard et al., 2001; Peschard et al., 2004; Tsygankov et al., 2001). Cbl-b has originally been characterized as a negative regulator of the adaptive immune response (Bachmaier et al., 2000; Chiang et al., 2000). Both T cell receptor (TCR) signaling and B cell receptor (BCR) signaling are modulated by Cbl-b mediated ubiquitination of critical signaling molecules (Loeser et al., Cell cycle 2007; Kitaura et al., 2007). Genetic ablation of Cbl-b (Bachmaier et al., 2000; Chiang et al., 2000) or knock-in of a catalytically inactive Cbl-b^{C373A/C373A} variant (Paolino et al., 2011) has thus been reported to cause broad autoimmune manifestations, particularly after antigenic challenge. Recent observations uncovered additional roles for Cbl-b in NK cell-mediated anti-tumor immunity (Paolino et al., 2014), FcεR signaling in mast cells (Gustin et al., 2006; Qu et al., 2004), integrin signaling (Han et al., 2010), and DC functions (Wallner et al., 2013), extending the range of Cbl-b functions to cells of the innate immune system. Signaling molecules targeted by Cbl-b are often not specific to defined pathways, but can be used by multiple signaling modules.

Spleen tyrosine kinase (Syk) is a signaling protein targeted by Cbl-family proteins, especially Cbl-b. Canonical signaling induced by, amongst others, the C-type lectin receptors (CLRs) Dectin-1, -2, and -3 is critically dependent on activation of Syk (Gross et al., 2009; Mocsai et al., 2010). Syk is recruited to Dectin-1, Dectin-2/3, and Mincle receptor complexes and subsequently activated by both phosphorylation via Src family kinases and auto-phosphorylation (Deng et al., 2015). Activated Syk then triggers the inflammasome (Gross et al., 2009; Gringhuis et al., 2012; Zwolanek et al., 2014), canonical NF-κB signaling (LeibundGut-Landmann et al., 2007; Ruland, 2008), and production of reactive oxygen species (ROS) in monocytes, macrophages, neutrophils and dendritic cells (DCs) (Mocsai et al., 2010). Collectively, the activation of Syk by CLRs causes an immediate activation of fungicidal pathways, as well as inducing cell type specific transcriptional changes tailored to eradicating local or systemic fungal infections. Syk signaling also plays a role in cancer.

According to the invention, it was found that administration of a Cbl-family protein inhibitor, especially a Cbl-b inhibitor, protected from morbidity and mortality induced by infection with Candida albicans, the most prevalent yeast pathogen, and similar fungal pathogens. It has also been found that administration of a Cbl-family protein inhibitor, especially a Cbl-b inhibitor, significantly reduced tumor growth. Thus, in a first aspect, the present invention provides a Cbl-family protein inhibitor, such as a Cbl-b inhibitor for use in the prevention or treatment of a fungal infection or cancer in a patient, wherein the fungal infection is a yeast infection. The patient may be in need of a therapy, e.g. having disease symptoms or an adverse prognosis.

The Cbl-family genes and their gene products are well described in the art (see for instance also UniGene Id. Mm.328206 and Hs.430589 for Cbl-b). Cbl-family protein sequences are published e.g. for Cbl-b in GenBank database under acc. no. NP_001028410.1 (Mus musculus) and NP_733762.2 (Homo sapiens). Cbl-family protein inhibitors are known in the art. Anti-Cbl-family protein antibodies, siRNAs and antisense inhibitors suitable as Cbl-family protein inhibitors are commercially available. Specifically, siRNAs suitable for reduction or inhibition of Cbl-family protein expression and thus also of Cbl-family function are disclosed for example in US 2007/0054355 A1, US 2010/0260808 A and US 2014/0010781 A (all incorporated herein by reference). Cbl-family member inhibitory peptides are disclosed in Kawai 2015, Kumar 2012 and Bechara 2013.

Any Cbl-family protein inhibitor, such as a Cbl-b inhibitor can be used according to the present invention. In one embodiment, the Cbl-family protein inhibitor is selected from nucleic acids targeting a Cbl-family protein, e.g. Cbl-b, such as Cbl-family gene expression inhibiting nucleic acids, siRNA, shRNA, miRNA.

The Cbl-family contains the members c-Cbl (or Cbl), Cbl-b and Cbl-c (or Cbl-3). Preferably Cbl-b is inhibited or the target of the Cbl-family binding peptide. In further embodiments, any one of c-Cbl or Cbl-c is inhibited or the target of the Cbl-family binding peptide. Members of the Cbl-family are referred to herein as “Cbl-family-ligases”, “Cbl family ligases”, “Cbl proteins”, Cbl-family proteins” and “Cbl-family members”. In some embodiments, an inhibitor may bind to or inhibit more than one family member, such as c-Cbl and Cbl-b. Cbl-b is the most preferred target and any aspects and preferred embodiments of the present invention read on inhibiting Cbl-b, e.g. a Cbl-family protein inhibitor may be a Cbl-b inhibitor and a Cbl-family TKB domain binding peptide may be a Cbl-b binding peptide (that binds the TKB domain of Cbl-b) in all aspects and embodiments of the invention.

It has been shown that the immune response to several clinically relevant yeast infections such as Candida infections of the family Saccharomycetaceae are regulated by a Cbl-family member. Thus, in a preferred embodiment, the yeast infection is of a yeast of the family Saccharomycetaceae, more preferably a candidiasis or a cryptococcosis. More preferably, the fungal infection is a Candida albicans, Candida glabrata, Candida krusei or a Candida dubliniensis infection.

The present invention also relates to a Cbl-family protein inhibitor for use in the prevention or treatment of cancer and/or a fungal infection in a patient, wherein the Cbl-family protein inhibitor is a Cbl-family protein binding peptide of Syk, especially preferred a Cbl-b binding peptide of Syk. The Syk gene and its gene products are well known in the art (UniGene Id. Mm.375031, Rn.87407, Mmu.32294 and Hs.371720). Syk sequences are published for example in GeneBank database under acc. nr. NP_001185906.2 (Mus musculus), NP_036890.1 (Rattus norvegicus), NM_001261035.1 (Macaca mulatta) and NP_003168.2 (Homo sapiens). Fragments of Syk that contain the sequence responsible for binding to Cbl-family members (Cbl-family protein binding peptide) have been shown to be a particularly effective treatment agent for the therapeutic inhibition of Cbl-family proteins. In particular, the Cbl-family protein-binding peptide binds to the TKB domain of a Cbl-family member (a TBK (domain) binding peptide).

In a preference, the Cbl-family protein binding peptide comprises the phosphotyrosine and surrounding flanking regions of Syk (FIG. 11e, right part, showing a murine Syk peptide). In one embodiment of the invention, the TKB domain binding peptide has the following consensus sequence: SFNPYEPX₁X₂X₃ (SEQ ID NO: 1), wherein X₁, X₂ and X₃ are selected independently from any amino acid. The amino acids are preferably selected from proteinogenic amino acids, e.g. the peptide may comprise at least 50% proteinogenic amino acids. Also preferred is the inclusion of one or more non-proteinogenic amino acid, especially to modify stability and/or pharmacodynamics of the peptide in vivo. The Y (tyrosine) in this sequence is preferably phosphorylated. In mice, the homologous sequence is SFNPYEPTGG (SEQ ID NO: 2) having amino acids 313-322 of murine Syk (Syk³¹³⁻³²²). In rats, the homologous sequence is SFNPYEPTGG (SEQ ID NO: 2) in position 313-322 of Syk (Syk³¹³⁻³²²). In rhesus monkeys, the homologous sequence is SFNPYEPEVA (SEQ ID NO: 3) in position 319-328 of Syk (Syk³¹⁹⁻³²⁸). In humans, the homologous sequence is SFNPYEPELA (SEQ ID NO: 4) in position 296-305 of Syk (Syk²⁹⁶⁻³⁰⁵). Thus, in an especially preferred embodiment, the Cbl-family protein binding peptide comprises the sequence SFNPYEPTGG (SEQ ID NO: 2) or SFNPYEPEVA (SEQ ID NO: 3) or the sequence SFNPYEPELA (SEQ ID NO: 4). Preferably X₁ is T or E. Preferably X₂ is G, L or V. Preferably, X₃ is G, or A. Also variants of these sequences are possible, therefore, the invention also relates to providing or using peptides comprising a sequence selected from SFNPYEPX₁X₂X₃ (SEQ ID NO: 1), SFNPYEPTGG (SEQ ID NO: 2), SFNPYEPEVA (SEQ ID NO: 3) or SFNPYEPELA (SEQ ID NO: 4) with one or two amino acid deletions or substitutions at any position.

In an especially preferred embodiment of the invention, the Cbl-family protein binding peptide comprises the following consensus sequence: STVSFNPYEPX₁X₂X₃X₄WX₅ (SEQ ID NO: 5), wherein X₁, X₂, X₃, X₄ and X₅ are selected independently from any amino acid, preferably containing proteinogenic and/or non-proteinogenic amino acids as described above. In mice, the homologous sequence is STVSFNPYEPTGGPWG (SEQ ID NO: 6) in position 310-325 of Syk (Syk³¹⁰⁻³²⁵). In rats, the homologous sequence is STVSFNPYEPTGGAWG (SEQ ID NO: 7) in position 310-325 of Syk (Syk³¹⁰⁻³²⁵). In rhesus monkeys, the homologous sequence is STVSFNPYEPEVAPWA (SEQ ID NO: 8) in position 316-331 of Syk (Syk³¹⁶⁻³³¹). In humans, the homologous sequence is STVSFNPYEPELAPWA (SEQ ID NO: 9) in position 293-308 of Syk (Syk²⁹³⁻³⁰⁸). Thus, in a related embodiment, the Cbl-family protein binding peptide comprises, preferably consists of, the sequence STVSFNPYEPTGGPWG (SEQ ID NO: 6), STVSFNPYEPTGGAWG (SEQ ID NO: 7), STVSFNPYEPEVAPWA (SEQ ID NO: 8) or STVSFNPYEPELAPWA (SEQ ID NO: 9). Especially, the tyrosine (y) in the indicated sequence portion -PYE- in the disclosed sequences is phosphorylated. Preferably X₁ is T or E. Preferably X₂ is G, L or V. Preferably, X₃ is G, or A. Preferably, X₄ is P or A. Preferably, X₅ is G or A. Also, variants of these sequences are possible, therefore, the invention also relates to providing or using peptides comprising a sequence selected from STVSFNPYEPX₁X₂X₃X₄WX₅ (SEQ ID NO: 5), STVSFNPYEPTGGPWG (SEQ ID NO: 6), STVSFNPYEPTGGAWG (SEQ ID NO: 7), STVSFNPYEPEVAPWA (SEQ ID NO: 8) or STVSFNPYEPELAPWA (SEQ ID NO: 9) with one, two or three amino acid deletions or substitutions at any position.

Preferably the Cbl-family protein binding peptide (or its portion in the fusion peptide) is a short peptide with 5 to 100 amino acids in length, preferably 8 to 80 or 12 to 60 amino acids in length.

The entire fusion peptide in any embodiment can be a peptide with 15 to 500 amino acids in length, preferably 15 to 200 amino acids in length, even more preferred 18 to 100 amino acids in length.

The Cbl-family protein binding peptides that bind to the TKB domain of a Cbl-family member, thereby inhibiting its activity can be administered to a cell in any form, conjugate or composition. These forms, conjugates or compositions for administration allow entry of the Cbl-family protein peptide into a living cell, thereby passing a cell membrane. Preferred are conjugates with cell delivery or cell penetrating moieties, such as fusion peptides with cell penetrating peptides. Conjugates and fusion peptides may be connected by a linker moiety or linker peptide. Such linkers are usually small, e.g. less than 10 kDa, or less than 5 kDa, or in the range of 0, or 1 to 20 amino acids in length. Other forms for cellular administration include vesicles, micelles, liposomes or microsomes.

Interestingly, a fusion peptide binding to the TKB domain of Cbl-b (FIG. 11e) significantly improved immune function in response to Candida albicans stimulation (FIG. 11g,h; FIG. 12b) and cancer (FIG. 15a). Further effects were shown for both Cbl-b and c-Cbl in activating a T-cell response against tumor antigens. The present invention, in a further aspect, therefore relates to a fusion peptide comprising a cell delivery portion and a Cbl-b binding peptide (in particular as defined above), wherein the Cbl-family protein binding peptide binds to the TKB domain of a Cbl-family member, preferably Cbl-b and/or c-Cbl. In particular, the fusion peptide comprises or consists of a cell delivery portion and a Cbl-family protein binding peptide, wherein the Cbl-family protein binding peptide binds to the TKB domain of a Cbl-family member (hence the peptide is also referred to as Cbl-family TKB domain binding peptide). The cell delivery portion may be a cell penetrating peptide. Possible routes of cellular delivery are fusion of vesicles etc. with a cell membrane, penetration through the lipid bilayer, translocation or endocytosis.

Cell penetrating peptides (CPPs) are short peptides, usually cationic or amphipathic, that mediate the intracellular delivery of a variety of biological cargos (Bechara et al., 2013; Jones et al., 2005, en.wikipedia.org/wiki/Cell-penetrating peptide, all incorporated herein by reference). Peptides derived from proteins, chimeric peptides and synthetic CPPs are the three main classes of CPPs that can be distinguished (Bechara et al., 2013). Having important advantages such as low toxicity, CPPs have become common biotechnological tools for cross-cellular transport of biologically active peptides in vitro and in vivo (Jones et al., 2005; Lindsay et al., 2002). Thus, in a preferred embodiment, the cell delivery portion of the fusion peptide is a cell penetrating peptide. Important representatives of CPPs are Antennapedia, Tat, Transportan, dNP1, dNP2 (Lim et al., 2015) or Polyarginine. Antennapedia has been shown to be especially useful for in vitro peptide delivery, taking into account both the magnitude of uptake and cytotoxicity (Jones et al., 2005). Especially effective are CPPs comprising two, three, four, five, six, seven or more cations, especially cationic amino acid residues, such as H, K or R, preferably selected from K or R. Preferably, the CPP is selected from DRQIKIWFQNRRMKWKK (SEQ ID NO: 10), GRKKRRQRRRPPQ (SEQ ID NO: 11), GWTLNSAGYLLGKINLKALAALAKKIL (SEQ ID NO: 12), (KKDKKDERRX_{A}K)ₘ (SEQ ID NO: 13), wherein 1≤m≤5 and X_{A} is selected from K or R, and (KIKKX_{B}KKKGRK)ₘ (SEQ ID NO: 14), wherein 1≤m≤5 and X_{B} is selected from V, I or T, or (R)ₙ, wherein 6≤n≤12. These CPP sequences can be repeated, e.g. duplicated. A preferred CPP is KIKKTKKKGRKKIKKTKKKGRK (aa 1-22 of SEQ ID NO: 43). Preferably the CPP (or its portion in the fusion peptide) is a short peptide with 8 to 100 amino acids in length, preferably 10 to 80 or 12 to 60 amino acids in length.

In another preferred embodiment, the Cbl-family TKB domain binding peptide of the fusion peptide is of or derived from Syk, as described above. Preferably, the Cbl-family TKB domain binding peptide is of Syk and the peptide is a regulator of innate immunity. The peptide is preferably a regulator of innate anti-parasitic immunity or anti-fungal immunity or anti-cancer immunity.

Other proteins or peptides binding to the TKB domain of a Cbl-family member can be used, such as proteins or Cbl-family TKB domain binding peptides of the insulin receptor substrate 1 (IRS-1), a key intermediate of skeletal muscle growth regulated by insulin-like growth factor (IGF-1) (Nakao et al., 2009; Ohno et al., 2016) or the Zeta-associated protein of 70 kDa (Zap-70), playing a role in T-cell development and lymphocyte activation. In addition, residues located in receptor tyrosine kinases (RTKs) such as EGFR, Met and CSF1 receptors that interact with the TKB domain of a Cbl-family member can be used (Mancini et al., 2002; Nakao et al., 2009; Ohno et al., 2016; Peschard et al., 2001; Peschard et al., 2004; Tsygankov et al., 2001). Some of the TKB binding sites of Cbl-b share a sequence motif comprising the phosphotyrosine. Thus, the Cbl-family TKB domain binding peptide preferably has the sequence motif N/DXpY, wherein X is selected from any amino acid, preferably containing proteinogenic and/or non-proteinogenic amino acids as described above. N/D is either asparagine (N) or aspartic acid (D), and pY is phosphotyrosine. More preferably, the Cbl-family TKB domain binding peptide has the sequence motif N/DX₁pYX₂P (SEQ ID NO: 15), wherein X₁ and X₂ are selected from any amino acid, preferably containing proteinogenic and/or non-proteinogenic amino acids as described above. N/D is either asparagine (N) or aspartic acid (D), and pY is phosphotyrosine. Even more preferred, the Cbl-family TKB domain binding peptide has a sequence length of 5 to 20 amino acids and/or a sequence identity to Syk of at least 80%, preferably at least 90%. In a further embodiment, the Cbl-family TKB domain binding peptide is a fragment of Zap-70, or IRS-1. In Zap-70 of mice and humans, the homologous sequence of the sequence motif N/DX₁pYX₂P (SEQ ID NO: 15) is DGpYTP (SEQ ID NO: 16) in position 288-292 in mice (mouse Zap-70²⁸⁸⁻²⁹²) and in position 290-294 in humans (human Zap-70²⁹⁰⁻²⁹⁴). In IRS-1 of mice and humans, the homologous sequence of the sequence motif N/DX₁pYX₂P is DGpYMP (SEQ ID NO: 17) in position 606-610 in mice (mouse IRS-1⁶⁰⁶⁻⁶¹⁰) and in position 610-614 in humans (human IRS-1⁶¹⁰⁻⁶¹⁴). Thus, in a further preferred embodiment, the Cbl-family TKB domain binding peptide comprises or consists of DGpYTP or DGpYMP.

Preferably, the Cbl-family TKB domain binding peptide comprises a tyrosine, wherein the tyrosine is phosphorylated. Phosphorylating the tyrosine increases interaction with a Cbl-family member, especially Cbl-b, and is a preferred embodiment in all aspects of the invention.

Any of these peptides can be used as retro-inverso peptide. A retro-inverso peptide has its sequence reversed, i.e. the left-to-right written sequence is not read from N- to C-terminus but the sequence is read as from C- to N-terminus. A benefit of retro-inverso peptides is increased stability. Any peptide in retro-inverso sequence is in D-form, instead of the L-form (see Brugidou et al., 1995; Snyder et al., 2004; Tugyi et al., 2005; Wang et al., 2017). For example, the consensus sequence of SEQ ID NO: 1 (SFNPYEPX₁X₂X₃) turns into dX₃dX₂dX₁dPdEdYdPdNdFdS with “d” depicting the D-form of the amino acid (also referred to as retro-inverso-SFNPYEPX₁X₂X₃) or the cell penetrating peptide of SEQ ID NO: 10 (DRQIKIWFQNRRMKWKK) turns into dKdKdWdKdMdRdRdNdQdFdWdIdKdIdQdRdD (also referred to as retro-inverso-DRQIKIWFQNRRMKWKK). Other properties remain as before, i.e. the tyrosine is preferably phosphorylated and it can be included, attached or fused to other peptides (both normal or further retro-inverso peptides). It is not necessary to turn the entire sequence of a given peptide sequence into a retro-inverso-form, a one or a few amino acids are sufficient to achieve increased stability of the peptide. E.g the Cbl-family binding peptide or the cell penetrating peptide or both contain 1, 2, 3, 4, 5, 6 or more retro-inverso amino acids, preferably the CPP contains the retro-inverso amino acid or sequence. For conventional reasons, if one D-amino acid without further flanking D-amino acid is included, this amino acid is simply referred to as D-form since no sequence inversion occurs. If 2 or more D-amino acids are in succession, the sequence of these successive D-amino acids is reversed and this part is referred to as retro-inverso-form.

The invention further relates to a pharmaceutical composition, comprising the fusion peptide or the Cbl-family TKB domain binding peptide, and a pharmaceutically acceptable carrier. The carrier may promote cellular uptake of said peptide.

In one preferred embodiment, the pharmaceutically acceptable carrier is selected from vesicles, micelles, liposomes and microsomes. These may increase cellular uptake of the peptide, for therapeutic or also non-therapeutic uses. Pharmaceutical compositions can comprise pharmaceutically acceptable salts, buffers, tonicity agents or pharmaceutically acceptable carriers. Pharmaceutical carriers provide improved compatibility of the composition and enable improved solubility as well as improved bioavailability of the active ingredients. Examples are emulsifiers, thickening agents, redox components, starch, alcohol solutions, polyethylene glycol or lipids. In another preferred embodiment, the pharmaceutical composition is prepared or used for parenteral administration, especially intravenous, intraarterial, intramuscular, intrathecal, subcutaneous or intraperitoneal administration. In another preferred embodiment, the pharmaceutical composition is prepared or used for topical administration, preferably further comprises a skin penetration enhancer. Suitable skin penetration enhancers are selected from the group comprising C12-C15 alkyl benzoate, pentylene glycol, polyethylene glycol, ethoxydiglycol, dimethyl sulfoxide, sodium lauryl sulfate, polysorbate polyethylene sorbitan monolaurate, lecithin, and mixtures thereof. In another preferred embodiment, the pharmaceutical composition is prepared or used for intra-tumoral and/or peri-tumoral administration.

Preferably, the peptide or pharmaceutical composition is for use in therapy, wherein the peptide or composition is administered to a patient. More preferably, the peptide or pharmaceutical composition is for use in the prevention or treatment of a fungal infection and/or cancer in the patient.

Toll-like receptors (TLRs) and CLRs have been shown to recognize conserved structures of invading pathogens, termed pathogen-associated molecular patterns (PAMPs), and thus induce immune responses (Kawai and Akira 2010; Kawai and Akira, 2011). Chiba et al. (2014) demonstrated that the CLR Dectin-1 recognizes tumor cells and, as a consequence, activates innate immune cells for anti-tumor responses. Dectin-1 serves as receptor for glyco-epitopes specifically expressed by tumor cells. Due to the function of Cbl-b in negative regulation of proximal Dectin-1 signaling, the TKB-binding peptide enhances Dectin-1 signal transduction impacting on the immune recognition of tumor cells by the innate immune system. In addition to the increase in activation of the immune system by the inventive inhibition of Cbl-b, this mechanism further enhances anti-tumor immunity driven by the innate immune system. Therefore, in a further preferred embodiment of the invention, the peptide or pharmaceutical composition is for use in the prevention or treatment of tumors, cancer, viral infections, bacterial infections, parasitic infections, especially intracellular parasites, mycosis, or drug-induced immunosuppression in a patient. Especially preferred is the treatment of a tumor and cancer. It has been shown herein that the inventive Cbl-family TKB domain binding peptides reduce tumor growth and enhance anti-tumor reactivity of the immune system. Further disease or conditions to be treated are characterized by a congenital or acquired immune insufficiency, such as AIDS, multiple myeloma, chronic lymphatic leukemia, drug-induced immunosuppression or a cancer, optionally with the selection of disease-specific antigens. Treatment of a cancer involving solid tumors is preferred in particular.

N-glycan structures are highly expressed in tumor cells. In another preferred embodiment of the invention, the peptide or pharmaceutical composition is for use in the prevention or treatment of such tumors, wherein the tumor cells express N-glycan at high amounts. High levels of N-glycan amounts (molar) in a particular cell type are at least 1.5-fold of the N-glycans found in said non-tumor cell type. These non-tumor cell types are e.g. melanocytes, epithelial cells, lymphatic cells, mesenchymal cells, lung cells, blood cells, liver cells. According to preferred embodiments of the invention, the cancer or tumor disease to be treated according to the present invention is selected from ovarian cancer, testicular cancer, prostate cancer, breast cancer; cancer diseases of the digestive tract, in particular stomach cancer, colon cancer, rectal cancer, pancreatic cancer, esophageal cancer and liver cancer; kidney cancer, skin cancer, in particular melanoma, basal cell carcinoma and squamous cell carcinoma; neuroblastoma and glioblastoma, lung cancer, thyroid cancer, sarcoma, head and neck cancer, squamous cell carcinoma, lymphoma and leukemia (wherein the terms “cancer”, “tumor”, “carcinoma” etc. are always used interchangeably herein and refer to malignant diseases).

Preferably, the peptide or pharmaceutical composition for use is administered to the patient by intraperitoneal injection, comprising 1 mg to 500 mg of the fusion peptide or Cbl-family TKB domain binding peptide, preferably for up to 7 days, 14 days, 21 days or 28 days and/or daily. Preferred doses are also 5 mg to 200 mg and 10 mg to 100 mg.

Cbl-b is a negative regulator of the innate and adaptive immune system (FIG. 3). Thus, the Cbl-b binding peptide as defined herein can positively stimulate both innate and adaptive immune reactions when administered as a vaccine adjuvant. Specifically, the Cbl-b binding peptide as defined herein can lift checkpoint blockades during vaccination. Therefore, the present invention also provides a Cbl-b inhibitor or any other Cbl-family protein inhibitor for use as a vaccine adjuvant in a patient, wherein the Cbl-family protein inhibitor is a Cbl-family TKB domain binding peptide as defined herein. In one embodiment, the vaccination is against tumors, or against viral, bacterial or parasitic infections. In an associated embodiment, the invention provides a vaccine, comprising as an adjuvant a Cbl-family protein inhibitor. Preferably, the Cbl-family protein inhibitor is a Cbl-family TKB domain binding peptide of Syk, and at least one antigen.

“Vaccination” as used herein is not to be understood in the absolute sense—i.e., administration of an immunogen which leads to absolute protection by the immune system—but rather as immunological administration to increase protection by the immune system and/or to activate the immune system, in particular the cells thereof against the vaccine antigen. The inventive Cbl-family protein inhibitor, especially the inventive peptide, increase the immunoreactivity to a vaccine antigen in a patient and/or increasing the immunoreactivity per se in patient.

In a preferred embodiment, the patient to be treated is a mammal. In an even more preferred embodiment, the patient is a human. In another embodiment, the Cbl-family protein inhibitor for use as a vaccine adjuvant is administered to the patient together with the vaccine that comprises the antigen against which an immune reaction shall be increased by Cbl-family protein inhibition. In an alternative embodiment, the Cbl-family protein inhibitor is administered to the patient before and/or after administration of the vaccine.

It has been shown by the present invention that inhibition of a Cbl-family protein is effective in the treatment of both systemic and cutaneous fungal infections, preferably candidiasis (e.g. example 3, FIG. 5h). A cutaneous fungal infection can further be a dermaphytosis, and a systemic fungal infection can also be an aspergillosis. Cutaneous fungal infections can be treated by topical therapy. Topical anti-fungal administration forms include creams, ointments, gels, lotions, powders, solutions or sprays. It has been further demonstrated in the present invention that treatment with the fusion peptide as described herein is effective in the treatment of already established fungal sepsis (Example 6, FIGS. 10j,k and 12g-k). Fungal sepsis has been reported to be increasing and to be associated with considerable morbidity and mortality (Lepak and Andes, 2011). In a preferred embodiment, the fungal infection to be treated with the inventive Cbl-family protein inhibitor, peptide or composition is a dermatophyte or an aspergillus infection. Preferably, the fungal infection is acute, systemic or cutaneous.

Preferably, the patient has or is at risk of developing sepsis, especially sepsis due to a fungal infection (“fungal sepsis”). The inventive increase in immune stimulation, especially of immune reactions against pathogens, may prevent or reduce the risk of sepsis. In an acute sepsis, it can quicken immune clearance.

The disease or condition to be treated may be acute or chronic. Chronic conditions are defined to last at least 90 days. Acute treatment may commence on days 0 to 89 after onset of the disease or condition, preferably on days 4 to 30.

In preferred embodiments of the present invention, the reduction or inhibition of the function of Cbl-family member is transient, i.e. the function is only temporarily reduced and can therefore recover again, e.g. by consumption or degradation of inhibitors. The transient reduction of a Cbl-family in immune cells in vitro or in a patient can also be performed in a repetitive manner, e.g. until a therapeutic success has been achieved.

In a further preferred embodiment of the invention, the Cbl-family protein inhibitor or peptide or pharmaceutical composition is administered to the patient intravenously, intraarterially, intramuscularly, intravascularly, subcutaneously, or intraperitoneally. More preferably, the patient to be treated is a mammal. Even more preferably, the patient is a human.

In a further preference, the Cbl-family protein inhibitor or peptide for use is administered to the patient using a pharmaceutically acceptable carrier, especially vesicles, micelles, liposomes or microsomes.

The present invention therefore also relates to a method for regulating, especially increasing, innate immunity, comprising the inhibition of a Cbl-family protein at least in immune cells, e.g. in a patient as described above. Preferably, a fusion peptide or Cbl-family TKB domain binding peptide as disclosed herein are administered to the patient. More preferably, the innate immunity is innate anti-parasitic immunity, innate anti-fungal, or innate anti-tumor immunity.

Immune cells targeted or affected by the inventive treatment are preferably antigen-presenting cells, PBMCs (peripheral blood mononuclear cells), T-lymphocytes, B-lymphocytes, monocytes, macrophages and/or dendritic cells, in particular T-lymphocytes, CD8+ T-lymphocytes, CD4+ T-lymphocytes, in particular Th1, Th2, Th17, Tregs (regulatory T-cells) or CTL (cytotoxic T-cells) or NK cells.

The present invention further relates to a kit comprising the peptide and a pharmaceutically acceptable excipient. Preferably, the kit is suitable for intracellular administration in the patient. Any route of administration to the patient as described above may be selected. The kit may comprise a container comprising the compounds, e.g. peptides or Cbl-family protein inhibitor of the invention and auxiliary substances of a pharmaceutical composition or other reagents to treat cells, the kit may be used in any method or any uses of the invention. The container and/or the kit may also comprise a further immunostimulating substance (apart from the peptide of the invention), preferably cytokine(s) or ligands of other receptors (e.g., TLRs) or antibodies to surface molecules, preferably CD3 and/or CD28, to additionally enhance the stimulation. Likewise, the kit or the container may also comprise stabilizing components, media or buffers.

In the kit or in a combinatory treatment of the patient or of cells, in vitro or in vivo, the Cbl-family protein inhibitor of the invention may be combined with further immune stimulating compounds such as an immune cell-stimulatory cytokine, e.g. a cytokine selected from the common gamma-chain cytokines, in particular IL-2, IL-15 and IL-21; cytokines that stimulate both the cells of the adaptive and of the innate immune system, in particular IL-12, IL-23 and IL-27; effector cell cytokines, such as IL-1, IL-17 and IL-18; an interferon, in particular interferon-alpha; or an interferon stimulator; an antibody, in particular an antibody which recognizes tumor cell surface molecules or a TLR or PAMP receptor ligand, in particular agonists, preferably of TLR-1, TLR-2, TLR-3, TLR-7, TLR-8 and TLR-9. An immune cell-stimulatory cytokine, a cytokine of both the adaptive and the innate immune system, an effector cell cytokine or an interferon stimulator according to the present invention are preferably selected from the group comprising IL-1, IL-2, IL-3, IL-4, IL-5, IL-6, IL-7, IL-8, IL-9, IL-11, IL-12, IL-13, IL-14, IL-15, IL-16, IL-17a, IL-17f, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IFN-alpha, IFN-beta, IFN-gamma, IFN-lambda, TNF (formerly TNF-alpha) and Lymphotoxin-alpha (formerly TNF-beta). The terms “simultaneous” or “together with” or “in combination with” or “combined with” as used in the context of the administration of the substances according to the present invention refer to the administration of at least one immune stimulating compound other than the Cbl-family protein inhibitor. If the administration is carried out using a plurality of different pharmaceutical compositions, the co-administration may be conducted simultaneously or sequentially.

Cbl-family proteins, such as Cbl-b, act in innate immune cells, especially in macrophages and dendritic cells, in vitro and that inhibition of Cbl-b increased immune response or activity of immune cells. Thus the invention relates to activating immune cells. The immune cells may be antigen-presenting cells, PBMCs (peripheral blood mononuclear cells), T-lymphocytes, B-lymphocytes, monocytes, macrophages and/or dendritic cells, in particular T-lymphocytes, CD8+ T-lymphocytes, CD4+ T-lymphocytes, in particular Th1, Th2, Th17, Tregs (regulatory T-cells) or CTL (cytotoxic T-cells) or NK cells. Such cells can also be treated and stimulated with the inventive Cbl-family protein inhibitor. The present invention therefore provides an in vitro method for stimulating or activating an immune cell, comprising contacting or treating the cell with a fusion peptide or a Cbl-family TKB domain binding peptide as disclosed herein. Preferably, the method comprises the production of cytokines by the activated or stimulated immune cells. E.g., the produced cytokines are selected from IL-6, IL-1β, IL-23 and TNF.

The term “treating” or “treatment” as used herein means to cure an already present disease state or condition or to increase the likelihood of recovery from the disease state or condition. Treating can also include inhibiting, i.e. arresting the development of a disease state or condition, and ameliorating, i.e. causing regression of a disease. Treatment can be to ameliorate disease symptoms without curing a patient.

The term “preventing” or “prevention” as used herein does not mean to stop a disease state or condition from occurring in a patient or subject completely but may also refer to a reduced risk of developing a disease state or condition.

In a preference, the pharmaceutical composition of the present invention can further comprise one or more excipients pharmaceutically acceptable for parenteral administration. Suitable excipients are known to the person skilled in the art, for example water (especially water for injection), saline, Ringer's solution, dextrose solution, buffers, Hank solution, vesicle forming compounds (e.g. lipids), fixed oils, ethyl oleate, 5% dextrose in saline, substances that enhance isotonicity and chemical stability, buffers and preservatives. This pharmaceutical composition can (as a drug) be administered via appropriate procedures known to the skilled person to a patient in need thereof (i.e. a patient having or having the risk of developing the diseases or conditions mentioned herein). The preferred route of administration of said pharmaceutical composition is parenteral administration, especially through intravenous, intraarterial, intramuscular, intrathecal, subcutaneous or intraperitoneal administration. For parenteral administration, the pharmaceutical composition of the present invention is provided in injectable dosage unit form, e.g. as a solution, suspension or emulsion, formulated in conjunction with the above-defined pharmaceutically acceptable excipients. The dosage and method of administration, however, depends on the individual patient to be treated. Said pharmaceutical composition, can be administered in any suitable dosage known from other peptide dosage regimens or specifically evaluated and optimized for a given individual. For example, the active agent (i.e. the fusion peptide of the present invention or the Cbl-family TKB domain binding peptide) may be present in the pharmaceutical composition in an amount from 1 mg to 10 g, preferably 50 mg to 2 g, in particular 100 mg to 1 g. Usual dosages can also be determined on the basis of kg body weight of the patient, for example preferred dosages are in the range of 0.1 mg to 100 mg/kg body weight, especially 1 to 10 mg/kg body weight (per administration session). The administration may occur e.g. once daily, once every other day, once per week or once every two weeks. As the preferred mode of administration of said pharmaceutical composition, is parenteral administration, the pharmaceutical composition according to the present invention is preferably liquid or ready to be dissolved in liquid such sterile, de-ionised or distilled water or sterile isotonic phosphate-buffered saline (PBS). Preferably, 1000 μg (dry-weight) of such a composition comprises or consists of 0.1-990 μg, preferably 1-900 μg, more preferably 10-200 μg active agent according to the present invention, and optionally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 100 mg of such a dry composition is dissolved in sterile, de-ionised/distilled water or sterile isotonic phosphate-buffered saline (PBS) to yield a final volume of 0.1-100 ml, preferably 0.5-20 ml, more preferably 1-10 ml.

Herein, the term “binding” or “binds” entails that molecule A (such as a molecule binding to a Cbl-family protein, such as the Cbl-family TKB domain binding peptide) has a dissociation constant (also called “affinity”) in regard to molecule B (such as a Cbl-family protein like Cbl-b, specifically the TKB domain thereof) that is lower than (i.e. “stronger than”) 1000 nM, preferably lower than 100 nM, more preferably lower than 50 nM, even more preferably lower than 10 nM, especially lower than 5 nM.

Preferably, the patient to be prophylactically or therapeutically treated is a mammal, especially a human. Typically, the patient (who preferably has a fungal infection as defined herein, and especially has been diagnosed with a fungal infection as defined herein) is in need of the inventive treatment. In the context of the inventive treatment for prevention of a fungal infection, the patient may be a patient that does not have the fungal infection but is at risk of developing the fungal infection, such as an immune-compromised patient. In another embodiment, the patient (who e.g. has cancer as defined herein, and especially has been diagnosed with cancer as defined herein) is in need of the inventive treatment. In the context of the inventive treatment for prevention of cancer, the patient may be a patient that does not have cancer but is at risk of developing cancer.

Herein, the term “TKB binding peptide” refers to a Cbl family binding protein, such as Cbl-b binding peptide which binds to the TKB domain of a Cbl-family member, such as Cbl-b.

“Comprising” is used as an open-ended term and means that other elements may also be present. “Consisting of” is used as a closed term, wherein, accordingly the described element or group of elements of the invention consist only of the recited element(s). An embodiment of the invention that has been described as comprising a certain element or group or elements, if not stated otherwise, may also only consist of this element or group of elements.

The use of the word “a” or “an” when used in conjunction with the term “comprising” in the claims and/or the specification may mean “one”, but it is also consistent with the meaning of “one or more”, “at least one”, and “one or more than one”.

Following embodiments further define the present invention:

1. A fusion peptide comprising a cell delivery portion and a casitas b-lineage lymphoma-family (Cbl-family) TKB domain binding peptide. The Cbl-family TKB domain binding peptide binds to the tyrosine kinase binding (TKB) domain of Cbl-family protein. In all embodiments and further definitions 2-47 of the invention, the Cbl-family protein is preferably Cbl-b and the Cbl-family TKB domain binding peptide is a Cbl-b binding peptide that binds to the TKB domain of Cbl-b.
2. The fusion peptide according to 1, wherein the cell delivery portion is a cell penetrating peptide.
3. The fusion peptide according to 1 or 2, wherein the Cbl-family TKB domain binding peptide is a peptide fragment of spleen tyrosine kinase (Syk).
4. The fusion peptide according to any one of 1 to 3, wherein the Cbl-family TKB domain binding peptide has the sequence motif N/DXpY, wherein X is selected from any amino acid, N/D is either asparagine (N) or aspartic acid (D), and pY is phosphotyrosine.
5. The fusion peptide according to any one of 1 to 4, wherein the Cbl-family TKB domain binding peptide has the sequence motif N/DX₁pYX₂P, wherein X₁ and X₂ are selected from any amino acid, preferably containing proteinogenic and/or non-proteinogenic amino acids as described above, N/D is either asparagine (N) or aspartic acid (D), and pY is phosphotyrosine.
6. The fusion peptide according to any one of 1, 2, 4 and 5, wherein the Cbl-family TKB domain binding peptide is a peptide fragment of zeta-associated protein of 70 kDa (Zap-70) or insulin receptor substrate 1 (IRS-1).
7. The fusion peptide according to any one of 1 to 6, wherein the Cbl-family TKB domain binding peptide binds to a Cbl-family member, the peptide comprising a tyrosine, wherein the tyrosine is phosphorylated.
8. The fusion peptide according to any one of 1 to 7, comprises the following consensus sequence:

SFNPYEPX₁X₂X₃,

wherein X₁-X₃ are selected independently from any amino acid, preferably containing proteinogenic and/or non-proteinogenic amino acids as described above, more preferably wherein the Cbl-family TKB domain binding peptide is a peptide fragment of Syk.
9. The fusion peptide according to 8, wherein the Cbl-family TKB domain binding peptide comprises the sequence SFNPYEPTGG.
10. The fusion peptide according to 8, wherein wherein the Cbl-family TKB domain binding peptide comprises the sequence SFNPYEPTGG.
11. The fusion peptide according to 8, wherein the Cbl-family TKB domain binding peptide comprises the sequence SFNPYEPEVA.
12. The fusion peptide according to 8, wherein the Cbl-family TKB domain binding peptide comprises the sequence SFNPYEPELA.
13. The fusion peptide according to any one of 1 to 12, wherein the Cbl-family TKB domain binding peptide is from Syk and comprises, preferably consists of, the following consensus sequence:

STVSFNPYEPX₁X₂X₃X₄WX₅,

wherein X₁-X₅ are selected independently from any amino acid, preferably containing proteinogenic and/or non-proteinogenic amino acids as described above, more preferably wherein the Cbl-family TKB domain binding peptide is a peptide fragment of Syk.
14. The fusion peptide according to 13, wherein the Cbl-family TKB domain binding peptide comprises, preferably consists of, the sequence STVSFNPYEPTGGPWG.
15. The fusion peptide according to 13, wherein the Cbl-family TKB domain binding peptide comprises, preferably consists of, the sequence STVSFNPYEPTGGAWG.
16. The fusion peptide according to 13, wherein the Cbl-family TKB domain binding peptide comprises, preferably consists of, the sequence STVSFNPYEPEVAPWA.
17. The fusion peptide according to 13, wherein the Cbl-family TKB domain binding peptide comprises, preferably consists of, the sequence STVSFNPYEPELAPWA.
18. The fusion peptide according to any one of 8 to 17, wherein the tyrosine in said sequence is phosphorylated.
19. The fusion peptide according to any one of 2 to 18, wherein the cell penetrating peptide is selected from DRQIKIWFQNRRMKWKK, GRKKRRQRRRPPQ, GWTLNSAGYLLGKINLKALAALAKKIL, (KKDKKDERRX_{A}K)ₘ, wherein 1≤m≤5 and X_{A} is selected from K or R, and (KIKKX_{B}KKKGRK)ₘ, wherein 1≤m≤5 and X_{B} is selected from V, I or T, or (R)ₙ, wherein 6≤n≤12.
20. A fusion peptide comprising a cell delivery portion as defined in any one of 1 to 19 and a Cbl-family TKB domain binding peptide, wherein the Cbl-family TKB domain binding peptide is an intramer or aptamer, wherein the Cbl-family TKB domain binding peptide inhibits a Cbl-family protein such as Cbl-b, preferably wherein the Cbl-family TKB domain binding peptide binds to the TKB domain of a Cbl-family protein.
21. The peptide according to any one of 1 to 20, wherein the Cbl-family TKB domain binding peptide is of Syk and wherein the peptide is a regulator of innate immunity.
22. The peptide according to any one of 1 to 21, wherein the Cbl-family TKB domain binding peptide is of Syk and wherein the peptide is a regulator of innate anti-parasitic, anti-fungal or anti-tumor immunity.
23. A pharmaceutical composition, comprising the fusion peptide or the Cbl-family TKB domain binding peptide as set forth in any one of 1 to 22, and a pharmaceutically acceptable carrier promoting cellular uptake of said peptide.
24. The pharmaceutical composition according to 23, wherein the pharmaceutically acceptable carrier is selected from vesicles, micelles, liposomes and microsomes.
25. The pharmaceutical composition according to 23 or 24, prepared for parenteral administration, especially intravenous, intraarterial, intramuscular, intrathecal, subcutaneous or intraperitoneal administration.
26. The pharmaceutical composition according to 23 or 24, prepared for topical administration, preferably further comprising a skin penetration enhancer.
27. The peptide or pharmaceutical composition according to any one of 1 to 26, for use in therapy, wherein the peptide or composition is administered to a patient.
28. The peptide or pharmaceutical composition for use according to 27 for use in the prevention or treatment of a fungal infection in the patient.
29. The peptide or pharmaceutical composition for use according to 27 or 28, wherein the peptide is a Cbl-family TKB domain binding peptide of Syk.
30. The peptide or pharmaceutical composition for use according to any one of 27 to 29 for use in the prevention or treatment of tumors, cancer, viral infections, bacterial infections, parasitic infections, especially intracellular parasites, mycosis, or drug-induced immunosuppression in a patient.
31. The peptide or pharmaceutical composition for use according to any one of 27 to 30, wherein the peptide is administered to the patient in a dose of 1 mg to 500 mg of the fusion peptide or Cbl-family TKB domain binding peptide, preferably for up to 7 days, 14 days, 21 days or 28 days and/or daily.
32. A Cbl-family protein inhibitor, preferably a Cbl-b inhibitor, for use in therapy in a patient as a vaccine adjuvant, preferably wherein the Cbl-family protein inhibitor is a Cbl-family TKB domain binding peptide, more preferably a Cbl-family TKB domain binding peptide of Syk.
33. A Cbl-family protein inhibitor for use in the prevention or treatment of a fungal infection in a patient, wherein the fungal infection is a yeast infection, preferably a candidiasis or a cryptococcosis.
34. A Cbl-family protein inhibitor for use in the prevention or treatment of a fungal infection in a patient, wherein the Cbl-family protein inhibitor comprises a Cbl-family TKB domain binding peptide of Syk.
35. The Cbl-family protein inhibitor or peptide or pharmaceutical composition for use according to any one of 27 to 34, wherein the fungal infection is a yeast infection, preferably a candidiasis, especially a Candida albicans, Candida glabrata, Candida krusei, or a Candida dubliniensis infection.
36. The Cbl-family protein inhibitor or peptide or pharmaceutical composition for use according to 27 to 34, wherein the fungal infection is a dermatophyte or an aspergillus infection.
37. The Cbl-family protein inhibitor or peptide or pharmaceutical composition for use according to any of 27 to 36, wherein the fungal infection is acute, systemic or cutaneous.
38. The Cbl-family protein inhibitor or peptide or pharmaceutical composition for use according to any one of 27 to 37, wherein the patient has or is at risk of developing fungal sepsis.
39. The Cbl-family protein inhibitor or peptide or pharmaceutical composition for use according to any one of 27 to 38, wherein the Cbl-family protein inhibitor, peptide or composition is administered to the patient intravenously, intraarterially, intramuscularly, intravascularly, subcutaneously, intraperitoneally, intraperitoneally or intrathecally.
40. The Cbl-family protein inhibitor, peptide or pharmaceutical composition for use according to any one of 27 to 39, wherein the patient is a mammal, preferably a human.
41. The Cbl-family protein inhibitor or peptide for use according to any one of 27 to 40, wherein the inhibitor or peptide is administered to the patient using a pharmaceutically acceptable carrier, especially vesicles, micelles, liposomes or microsomes.
42. A vaccine, comprising as an adjuvant a Cbl-family protein inhibitor, preferably wherein the Cbl-family protein inhibitor comprises a Cbl-family protein binding peptide of Syk, and at least one antigen, preferably the Cbl-family protein inhibitor being defined as in any one of 1 to 22 and 27 to 40.
43. A method for regulating, especially increasing, innate immunity, comprising the inhibition of a Cbl-family protein, preferably by administering to a patient the fusion peptide or a Cbl-family protein inhibitor as set forth in any one of 1 to 22 and 27 to 40.
44. The method according to 43, wherein the innate immunity is anti-parasitic, anti-fungal immunity, or anti-tumor immunity.
45. A kit comprising the peptide according to 1 to 31 and a pharmaceutically acceptable excipient, preferably suitable for intracellular administration in the patient.
46. An in vitro or in vivo method for stimulating or activating an immune cell, comprising contacting or treating the cell with a fusion peptide or a Cbl-family TKB domain binding peptide as set forth in any one of 1 to 22.
47. The in vitro or in vivo method for stimulating or activating an immune cell according to 46, wherein cytokines are produced, wherein the cytokines are preferably selected from IL-6, IL-1β, IL-23 and TNF.

The present invention is further illustrated by the following figures and examples, without being restricted thereto.

### FIGURES

FIG. 1: Cbl-b regulates anti-fungal immunity.

(a,b) Cbl-b^{+/+}, Cbl-b^{Δ/Δ}, and Cbl-b^{C373A/C373A} mice were infected intravenously with Candida albicans (10⁵ CFU/21.5 g body weight) and monitored for the indicated time periods. Plots depict (a) weight loss over time after infection as compared to starting weight (p values assessed by two-way ANOVA) or (b) survival over time after infection (p values assessed with log rank test). n=6 for Cbl-b^{+/+} and Cbl-b^{C373A/C373A}, n=7 for Cbl-b^{Δ/Δ} mice. (c) Candida albicans fungal load in depicted organs at day 7 after infection, plotted as colony forming units (CFU) per gram organ weight. Each dot represents an individual mouse. (d,e) Representative kidney sections from mice 7 days after systemic Candida albicans infection, stained with periodic-acid-Schiff (PAS) (d). Insets show regions of renal inflammation from boxed areas at increased magnification. (e) Combined inflammatory score based on the extent of renal immune cell infiltration, inflammation and tissue destruction. (f,g) Representative kidney sections from mice 7 days after Candida albicans infection, stained with Gomori methenamine silver (GMS) (f). Insets show regions of fungal growth from boxed areas at increased magnification. Yellow arrowhead indicates fungal hyphae. (g) Plot depicts intralesional fungal load, as scored by histopathologic assessment of GMS stained kidney sections. (h,i) Representative kidney sections stained for the neutrophil marker Ly-6G (h). Insets show regions of neutrophil infiltration from boxed areas. (i) Plot depicts the percentage of kidney area scored positive for Ly-6G staining using Definiens Tissue Studio software. For panels (d-g) n=4 for Cbl-b^{+/+}, and n=5 for Cbl-b^{Δ/Δ} mice; for panels (h,i) n=4 for Cbl-b^{+/+}, n=4 for Cbl-b^{Δ/Δ} mice; for panels (d-i) 4 sections per mouse were analyzed. For panels (c,e,g,i) data are shown as means±standard deviation. For all experiments, 1 representative experiment of 3-6 independent repeats is shown. *P<0.05, **P<0.01, ***P<0.001 as calculated with Student's t-test, unless stated otherwise.

FIG. 2: Candida ssp. and Listeria monocytogenes infection.

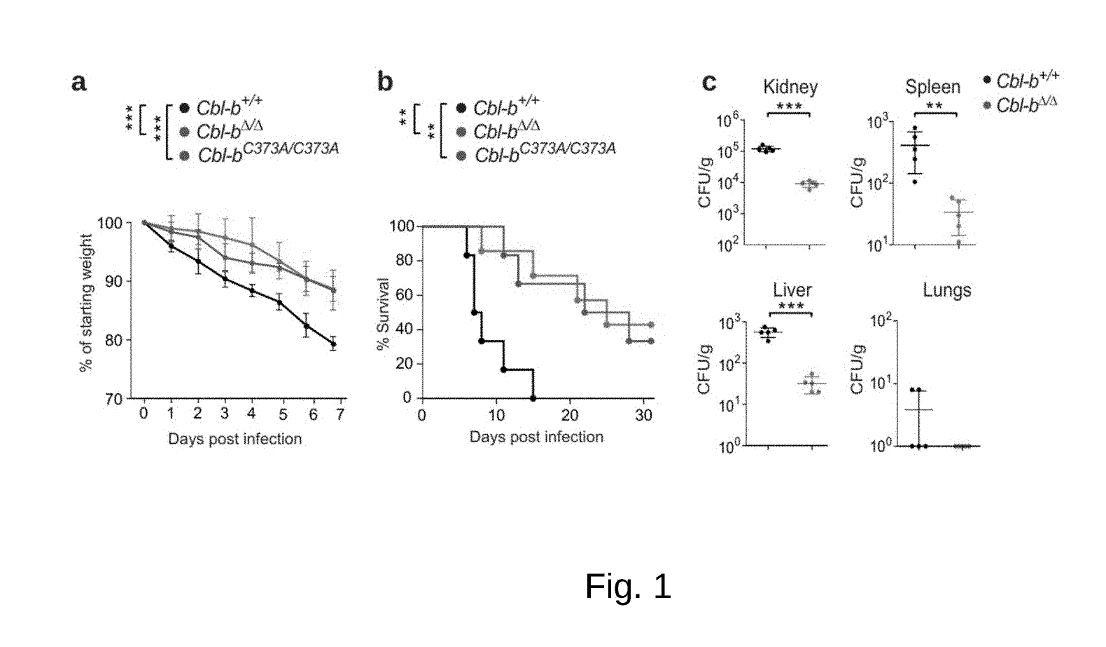

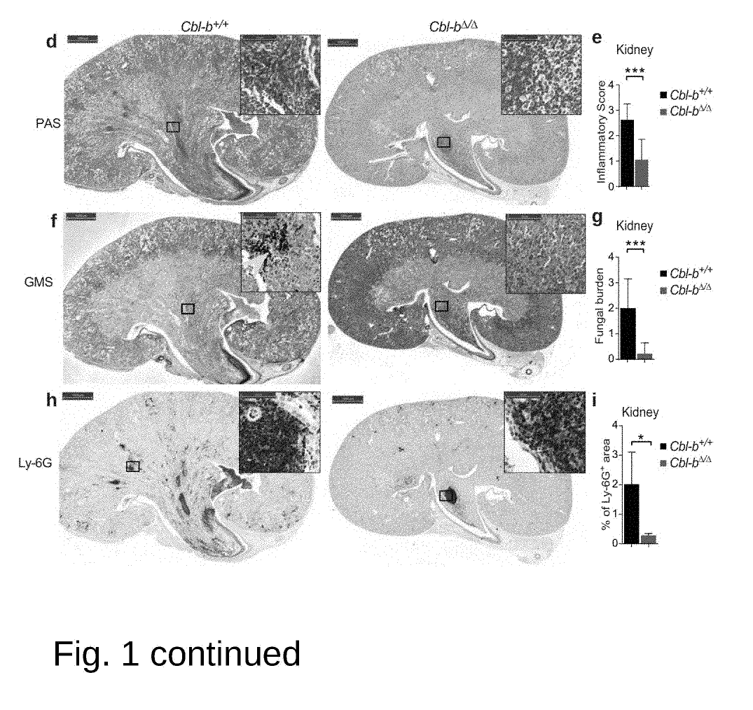

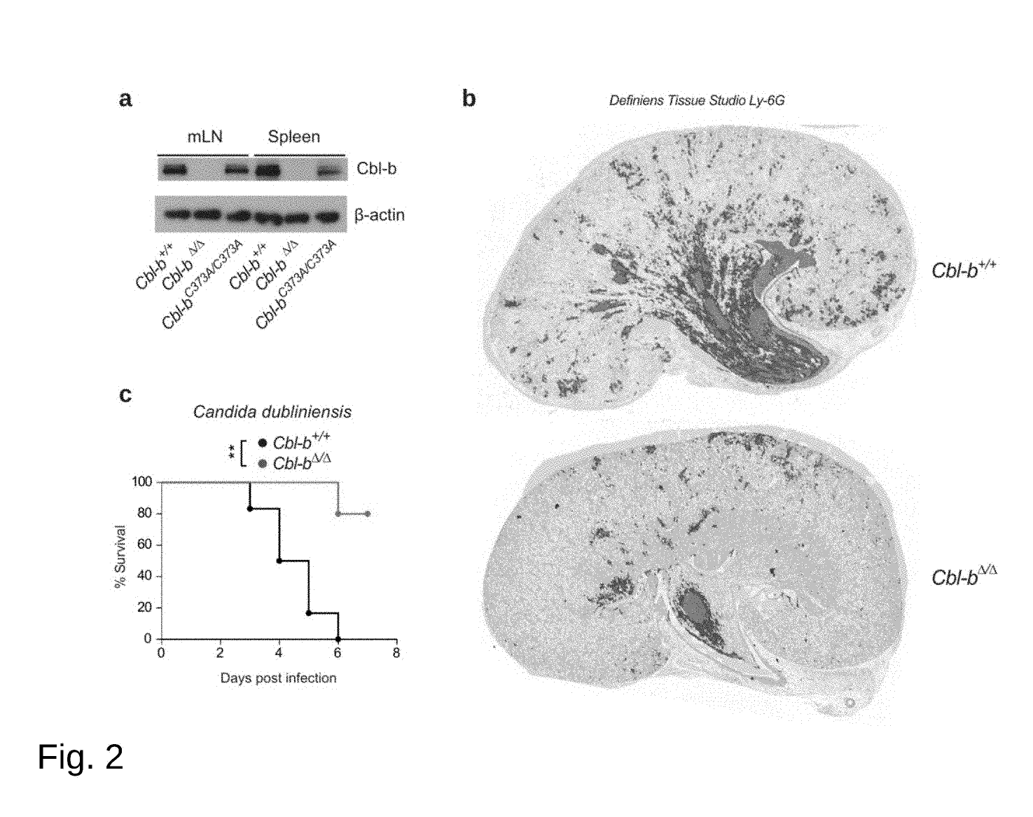

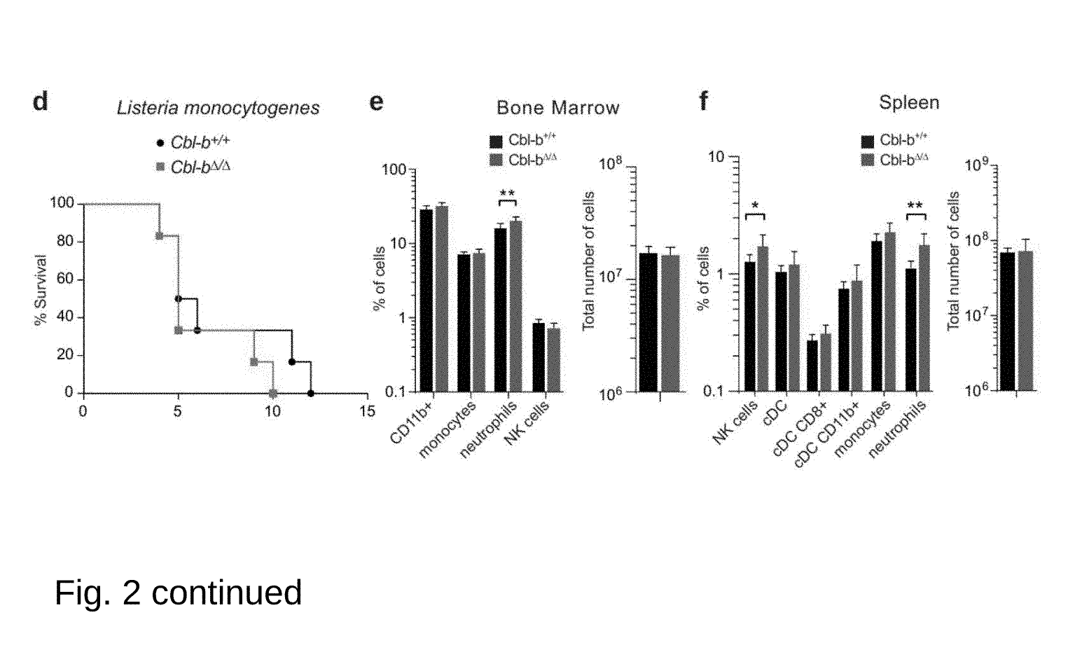

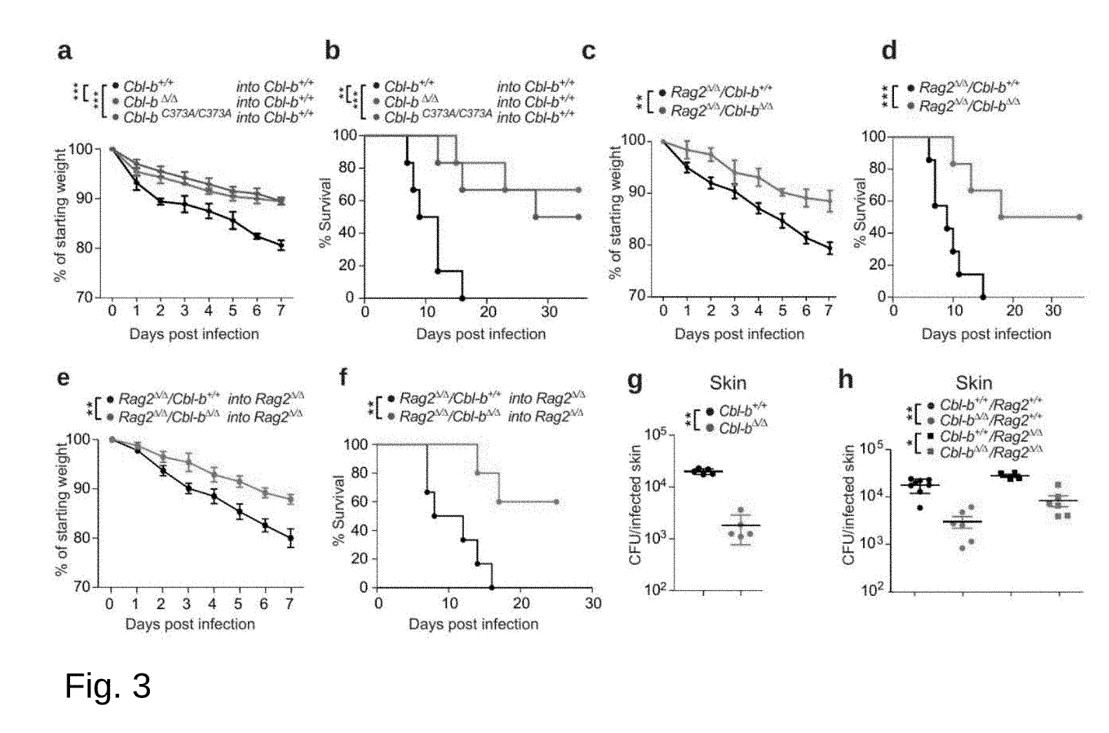

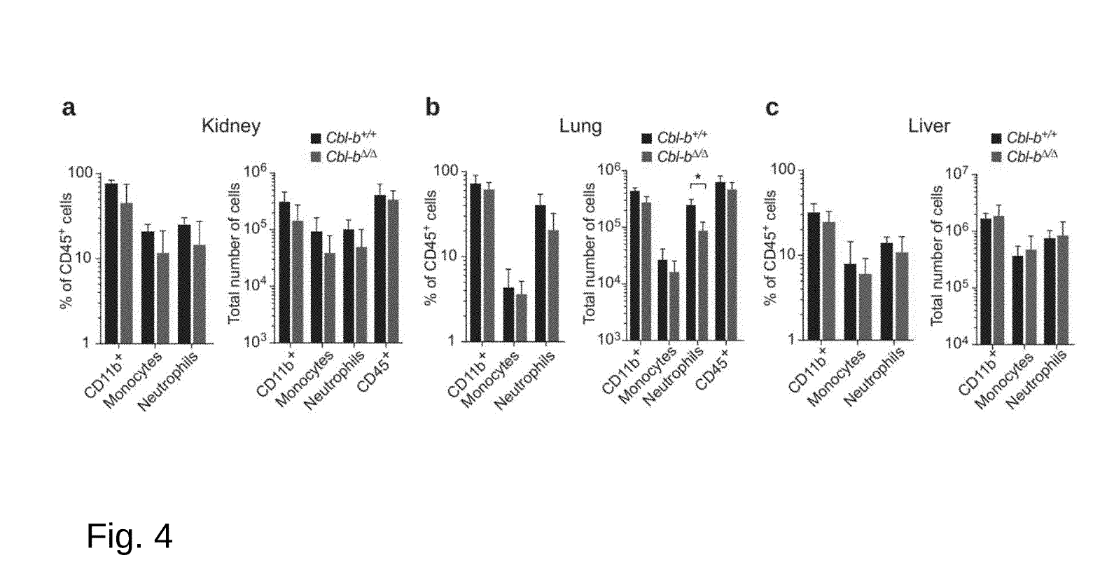

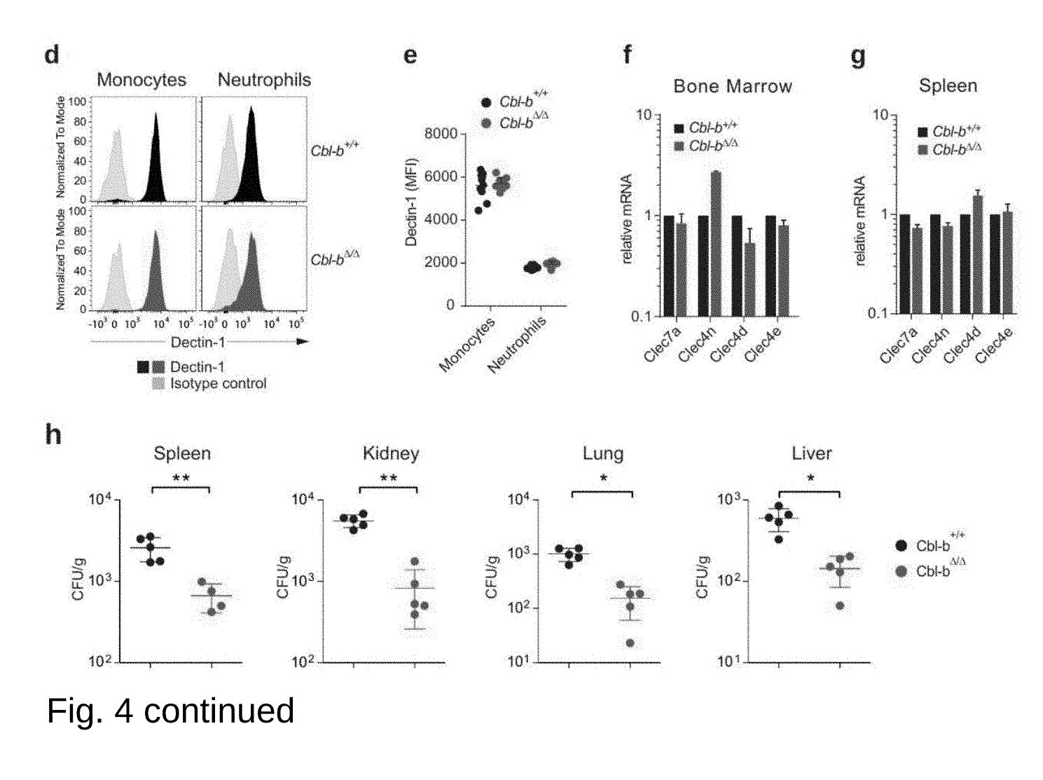

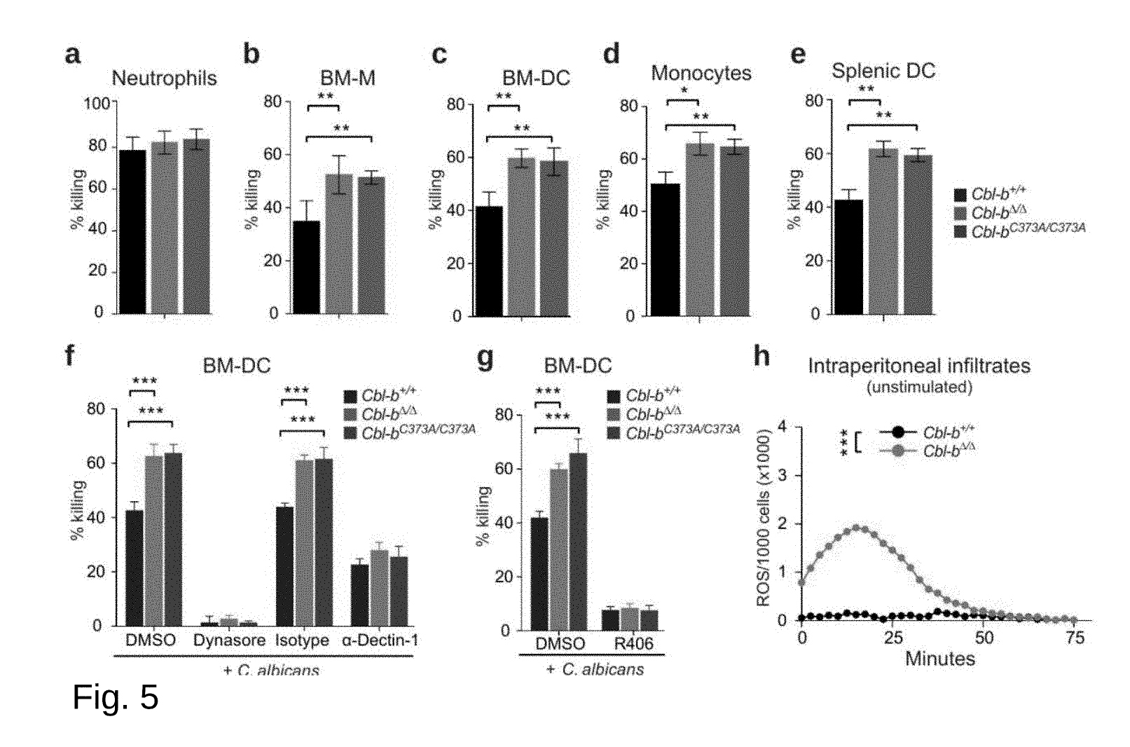

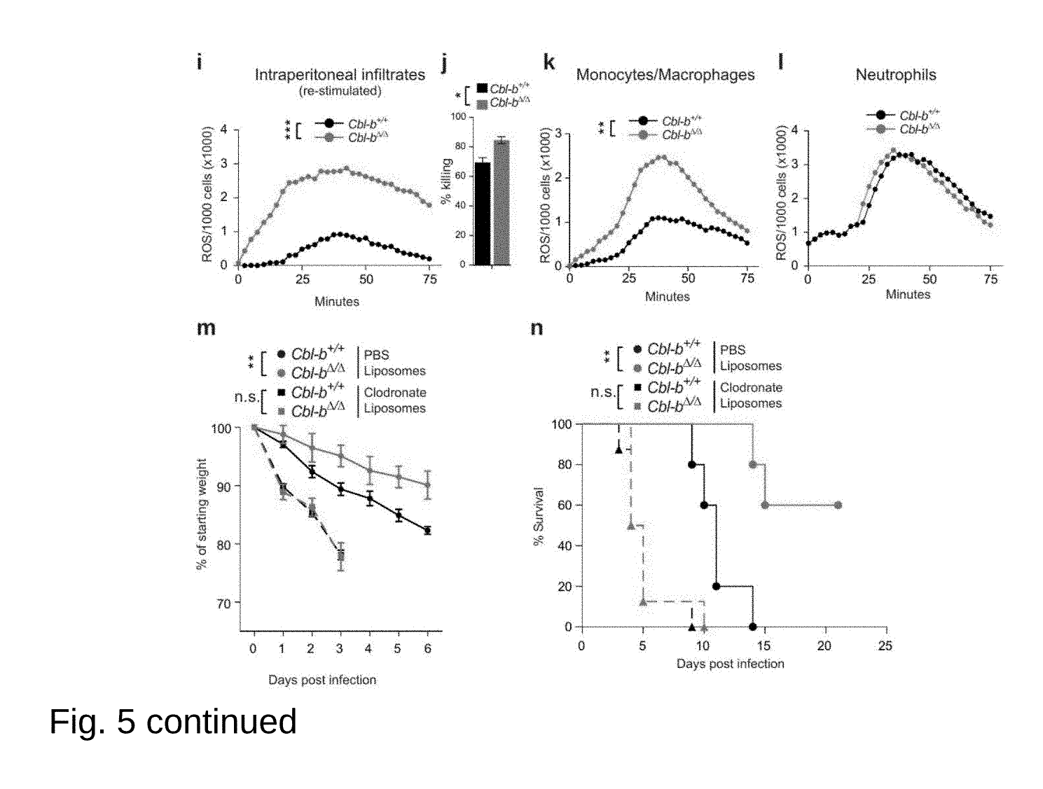

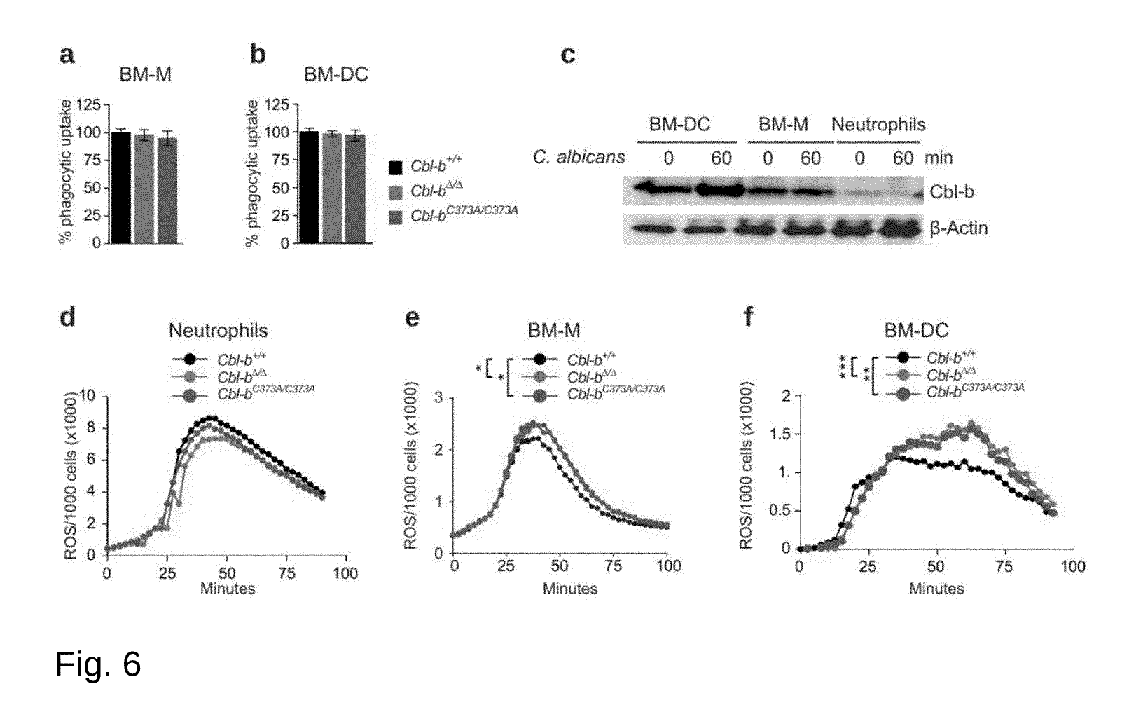

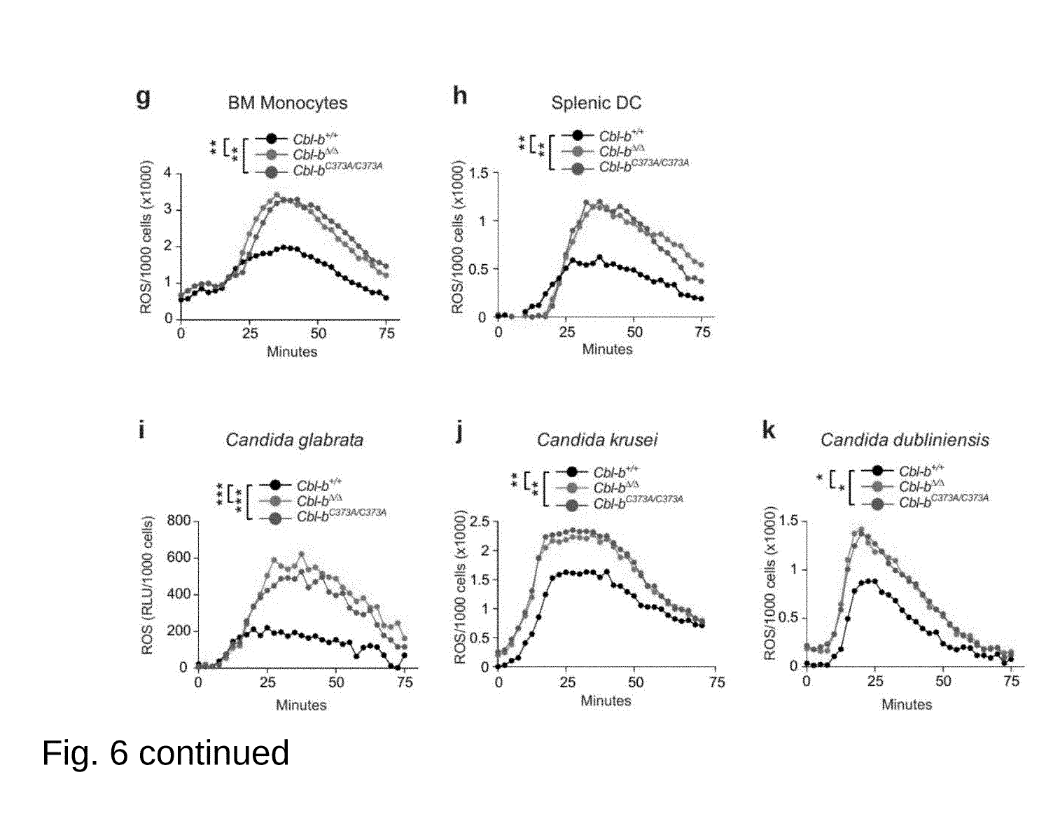

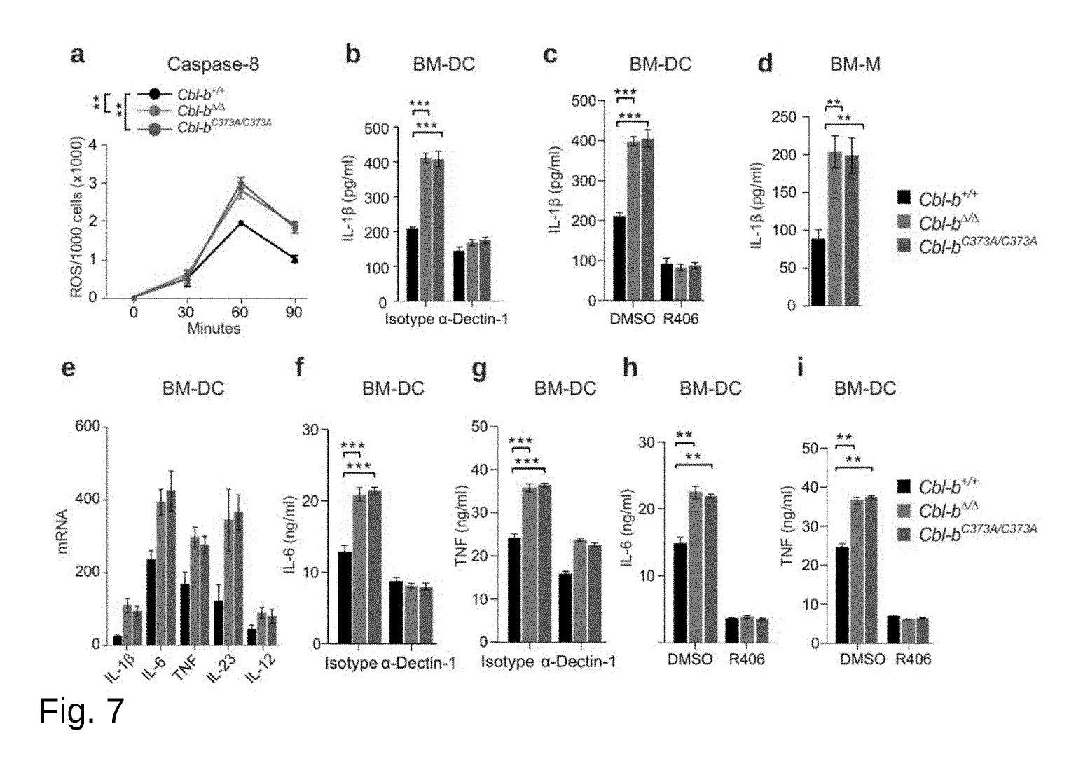

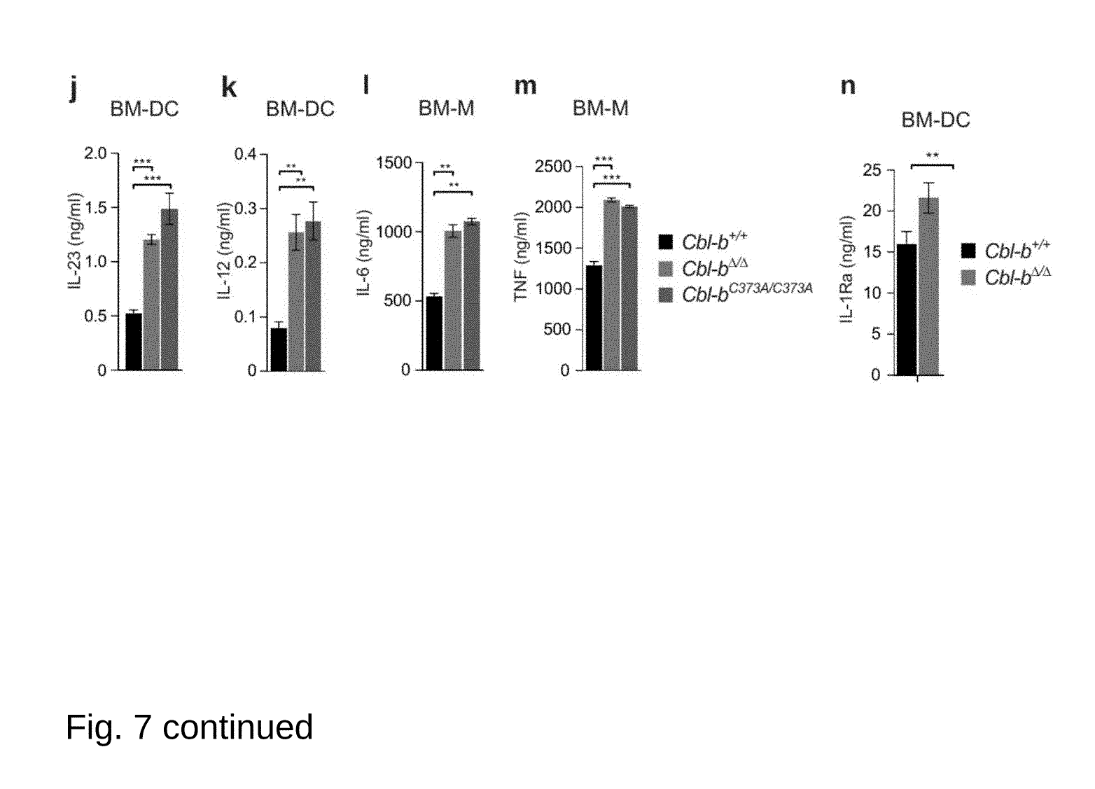

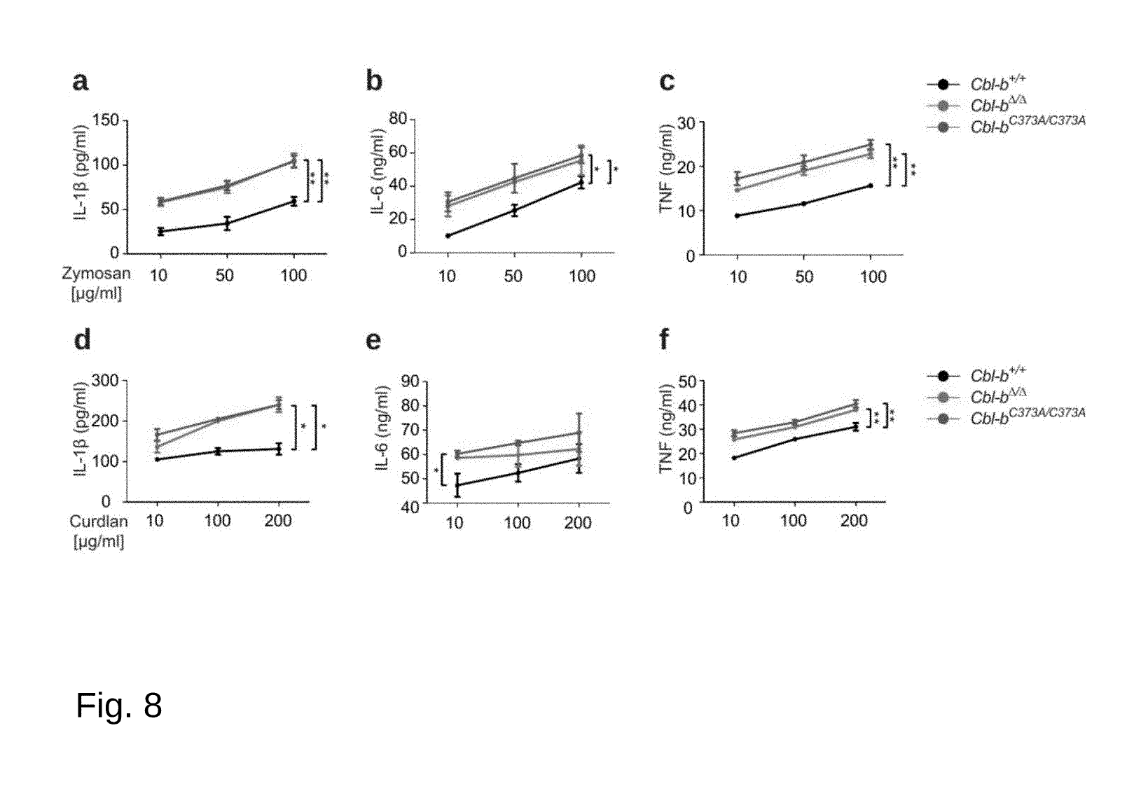

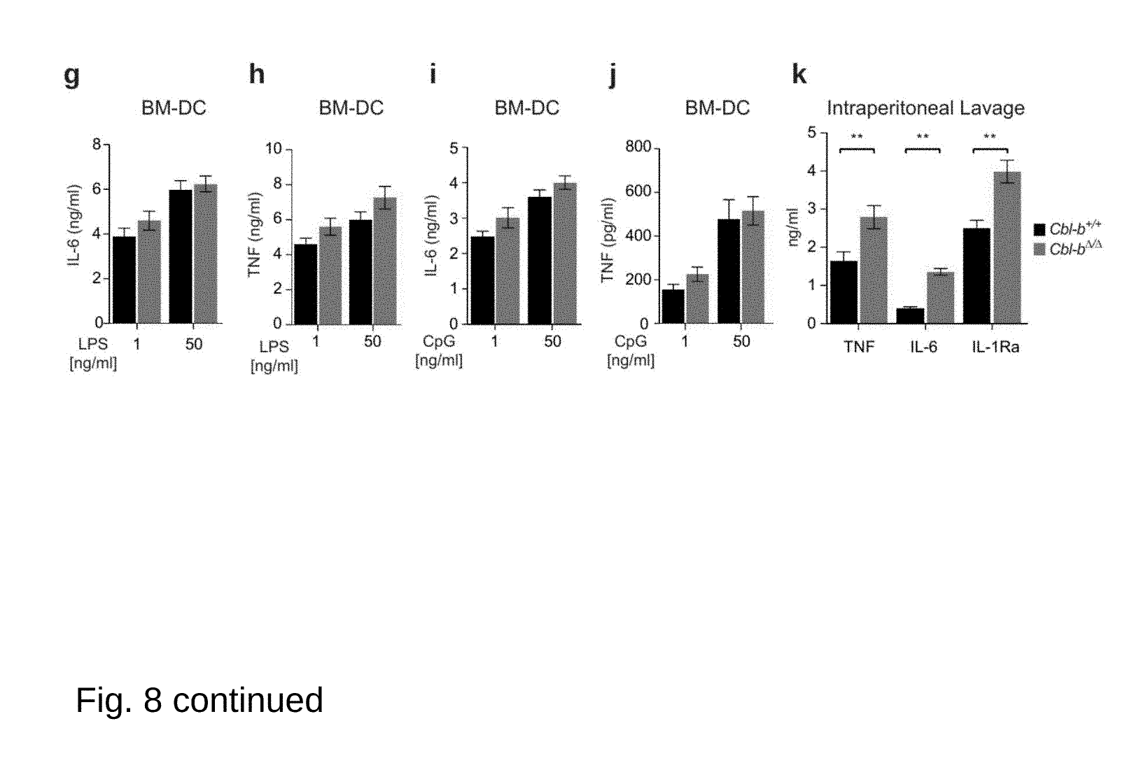

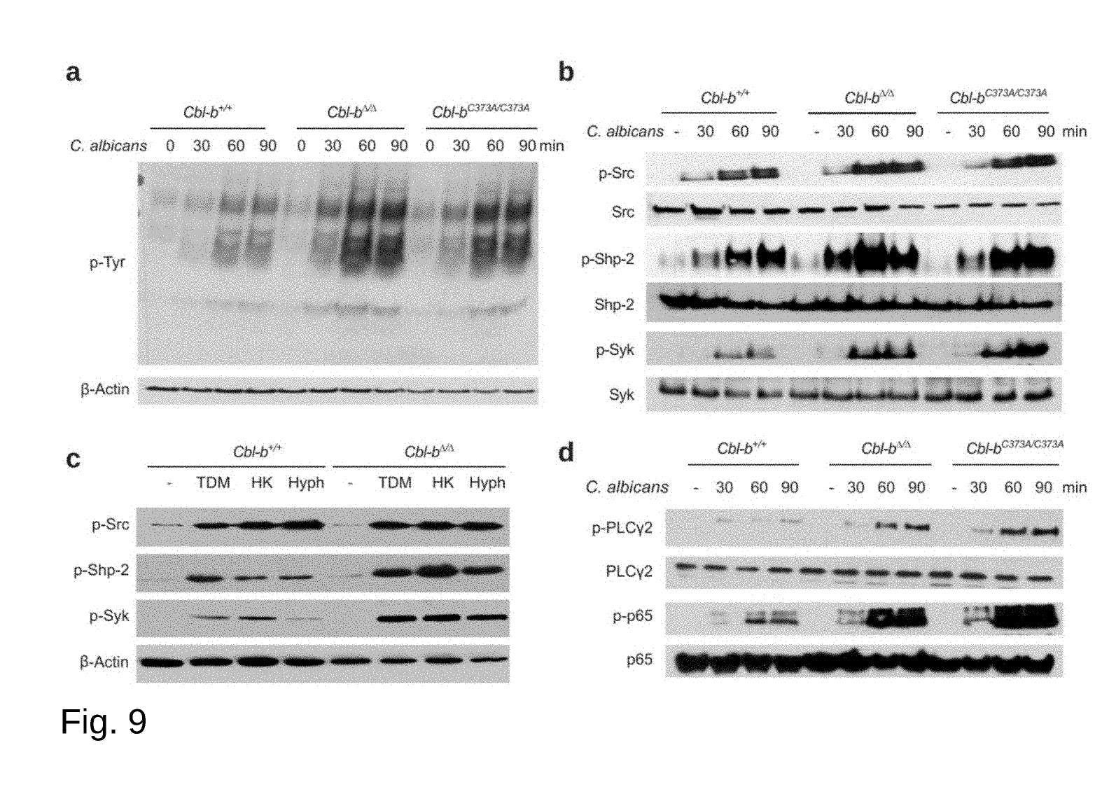

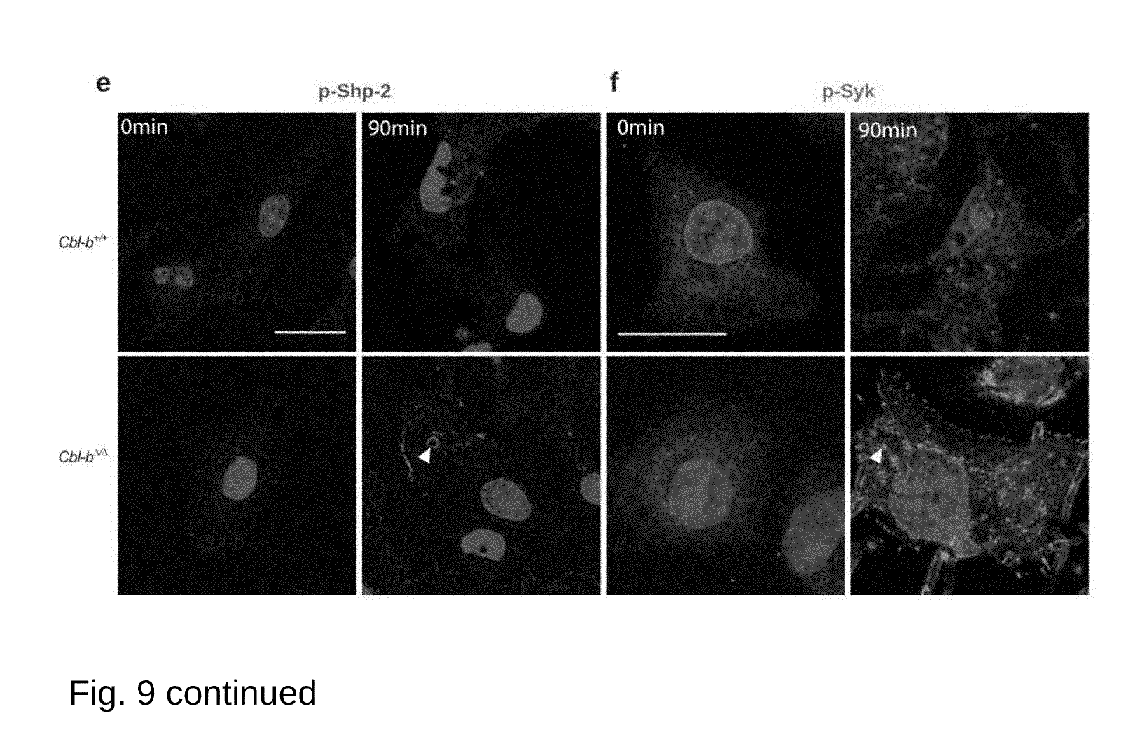

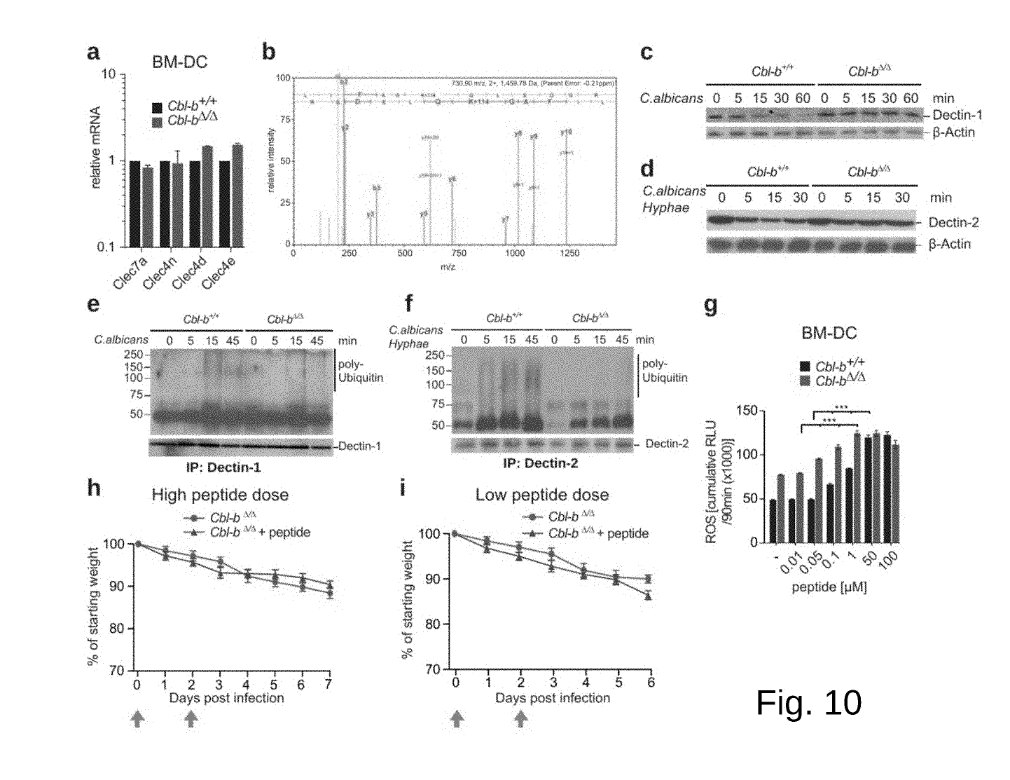

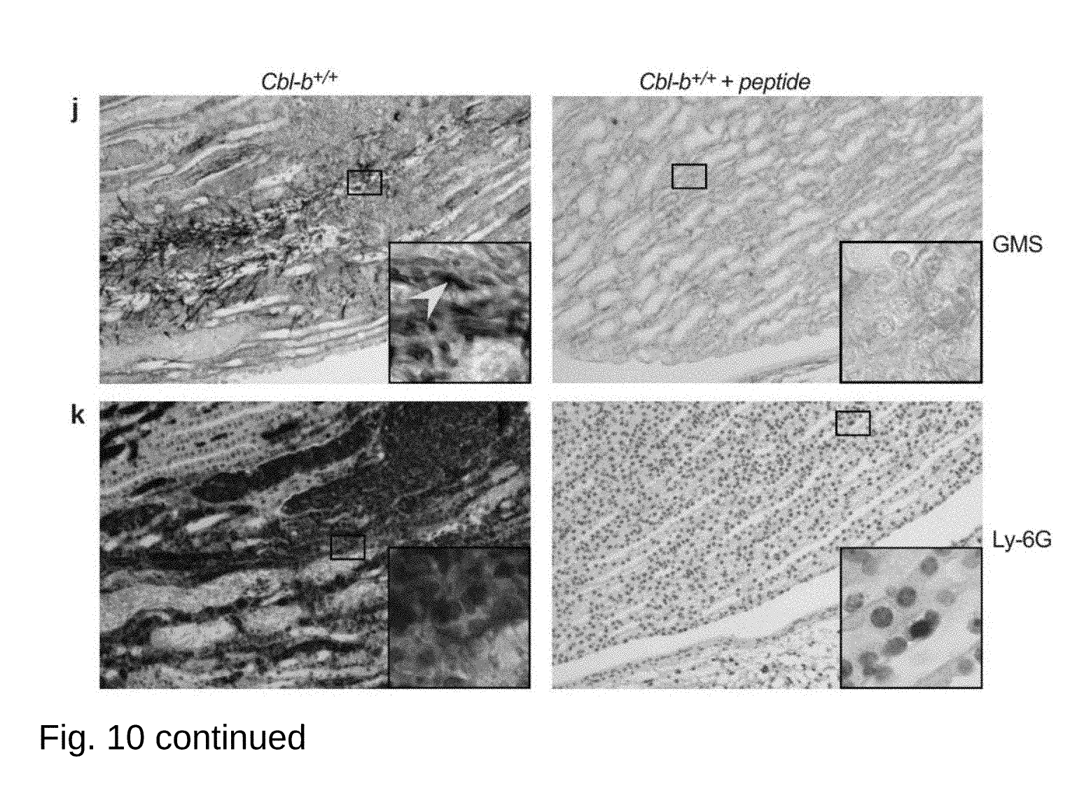

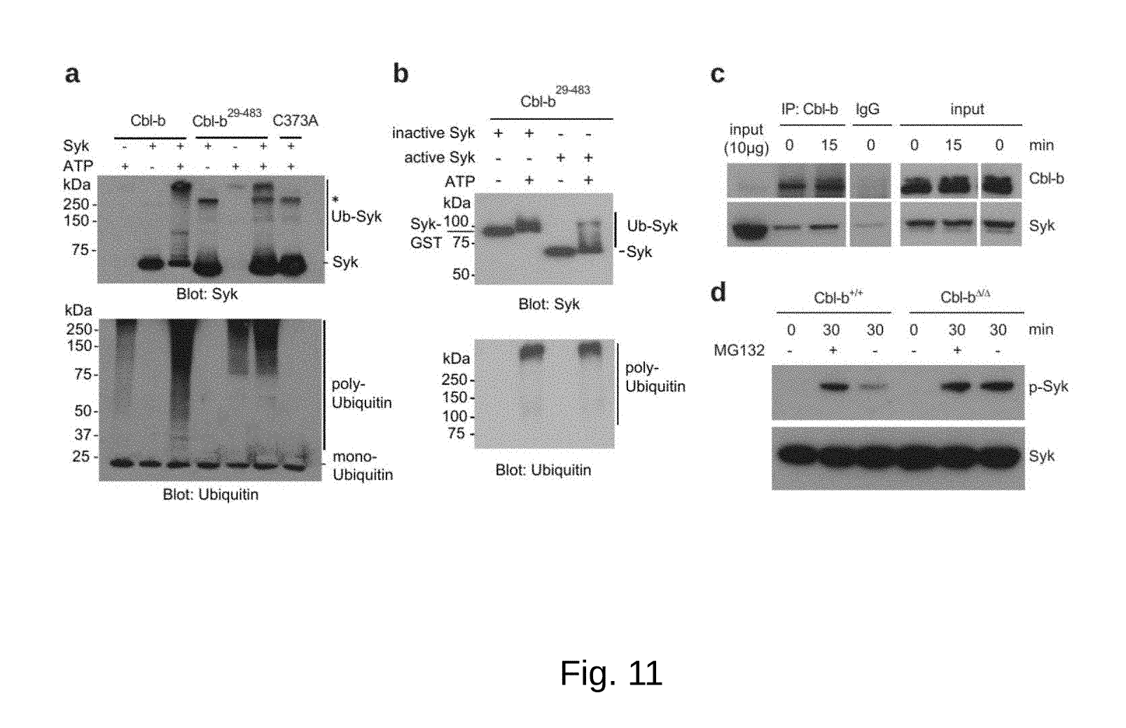

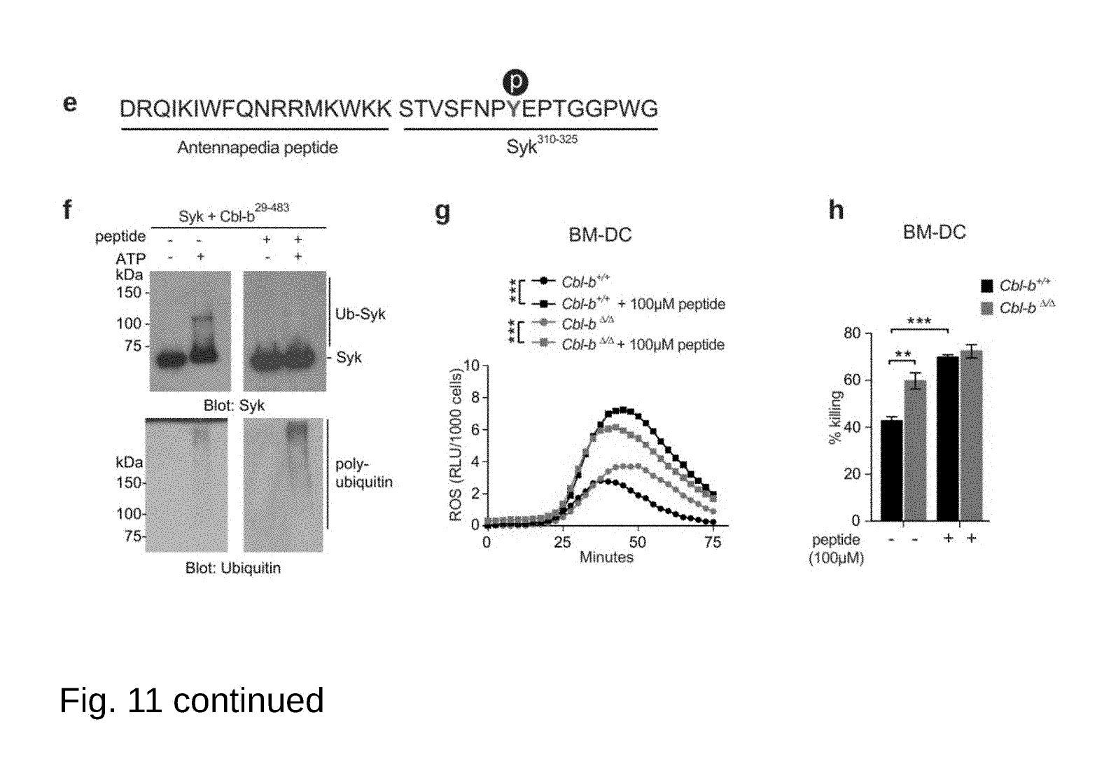

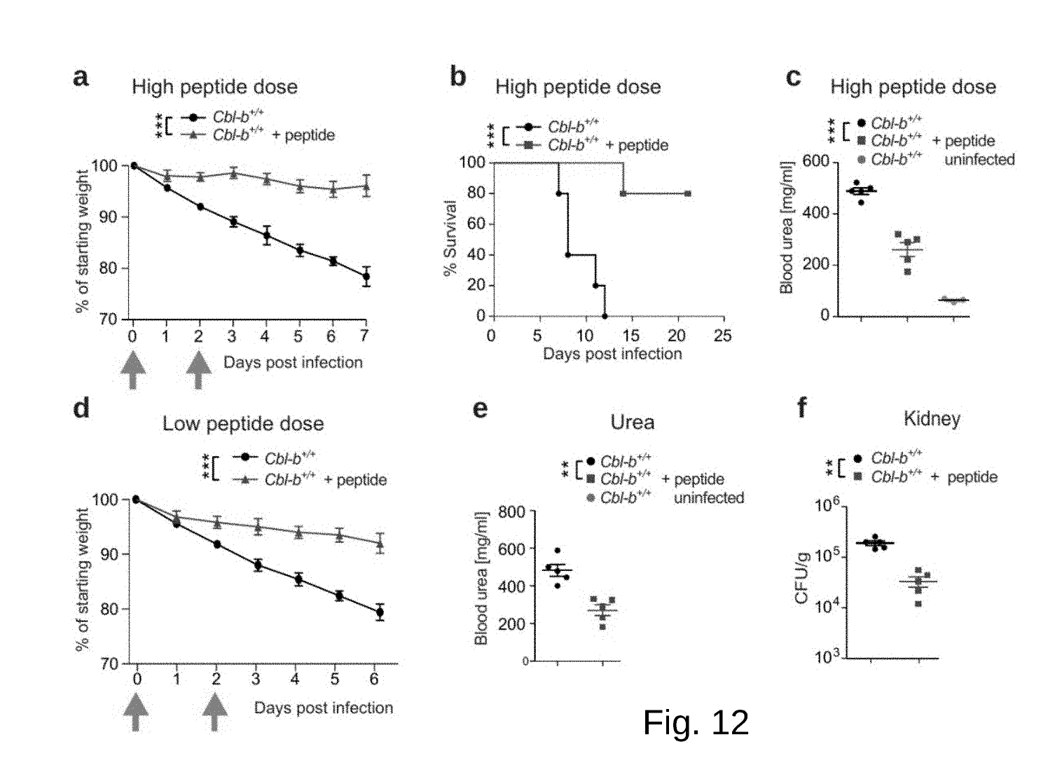

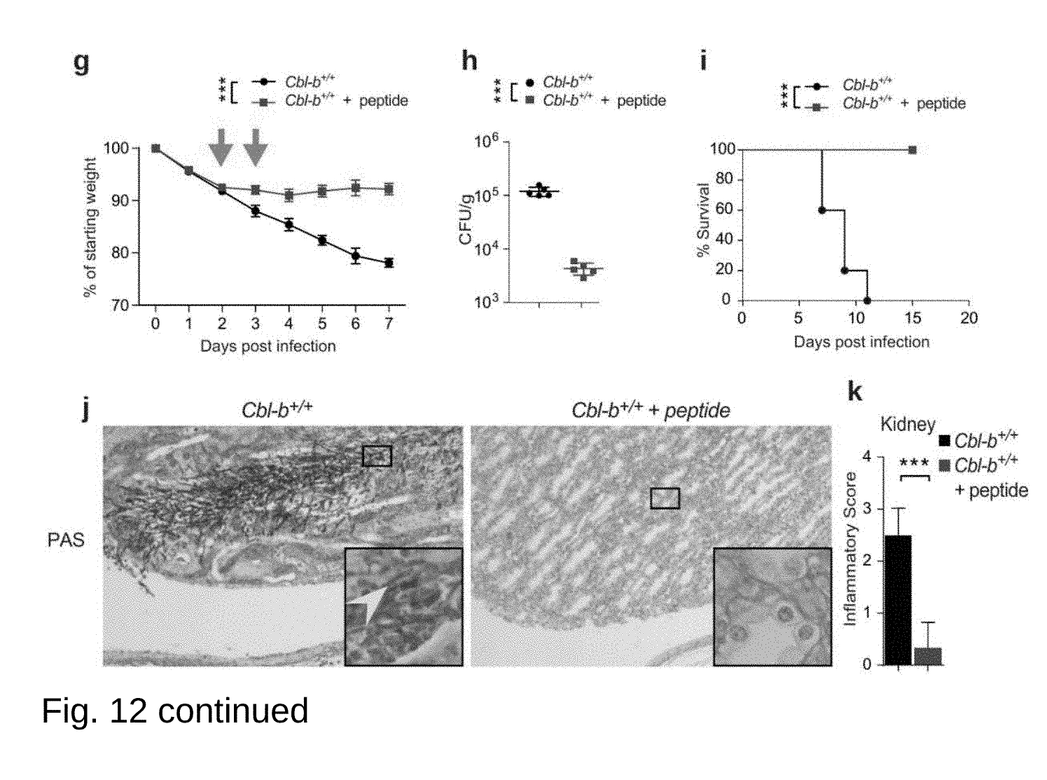

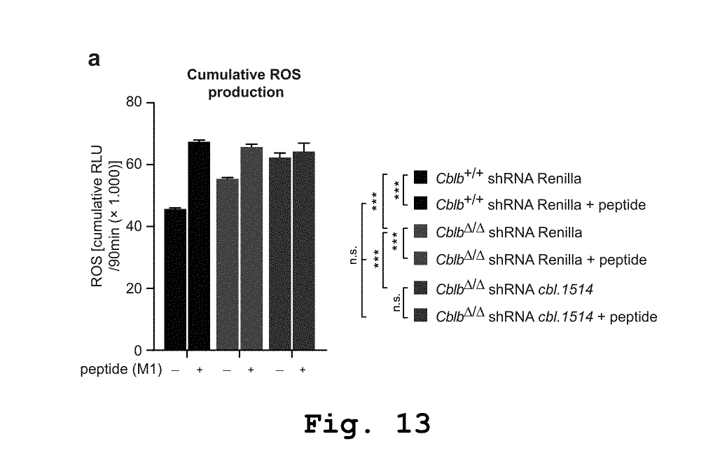

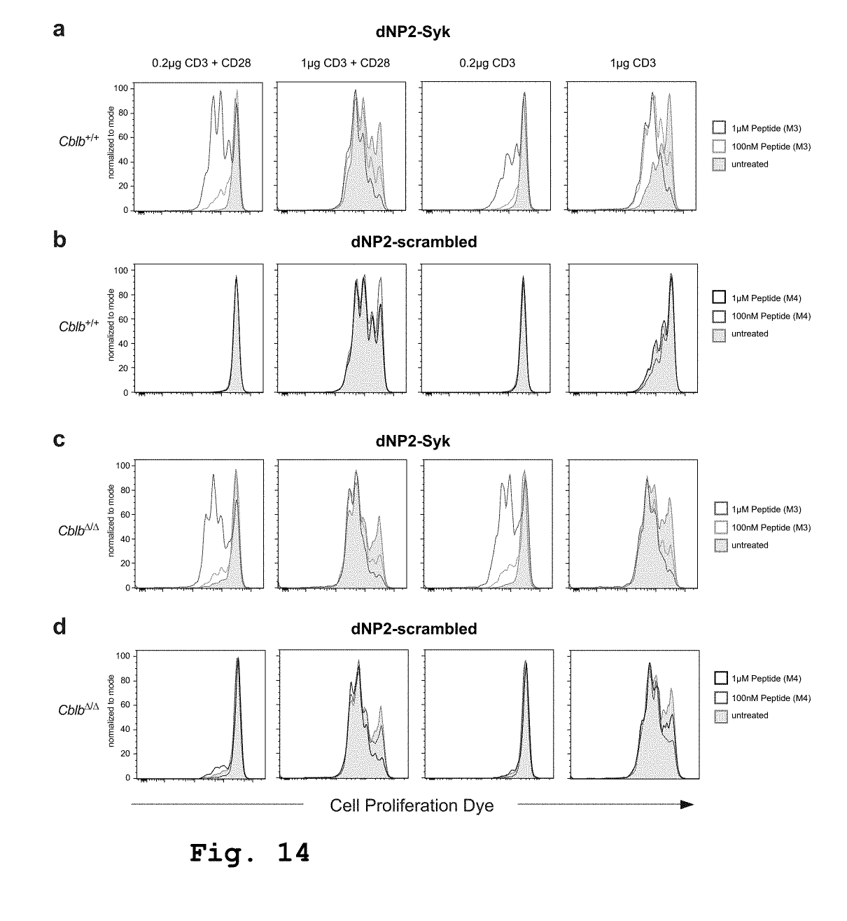

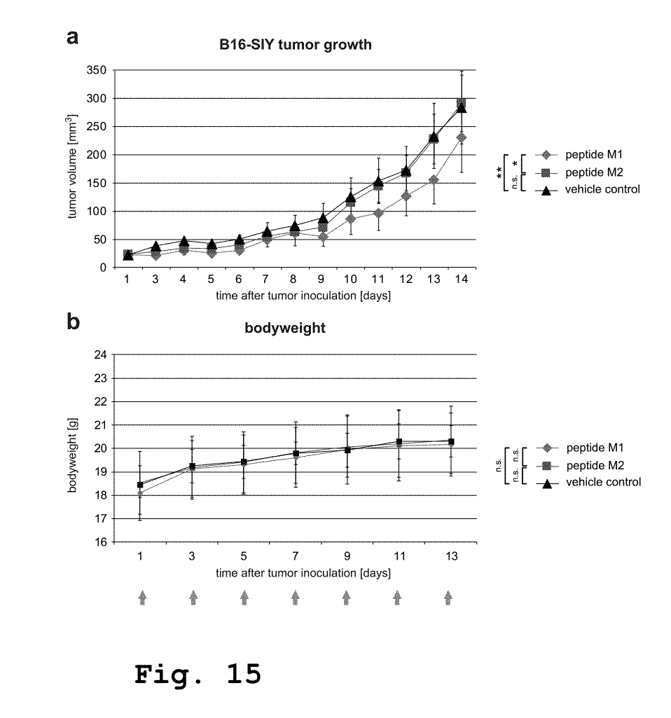

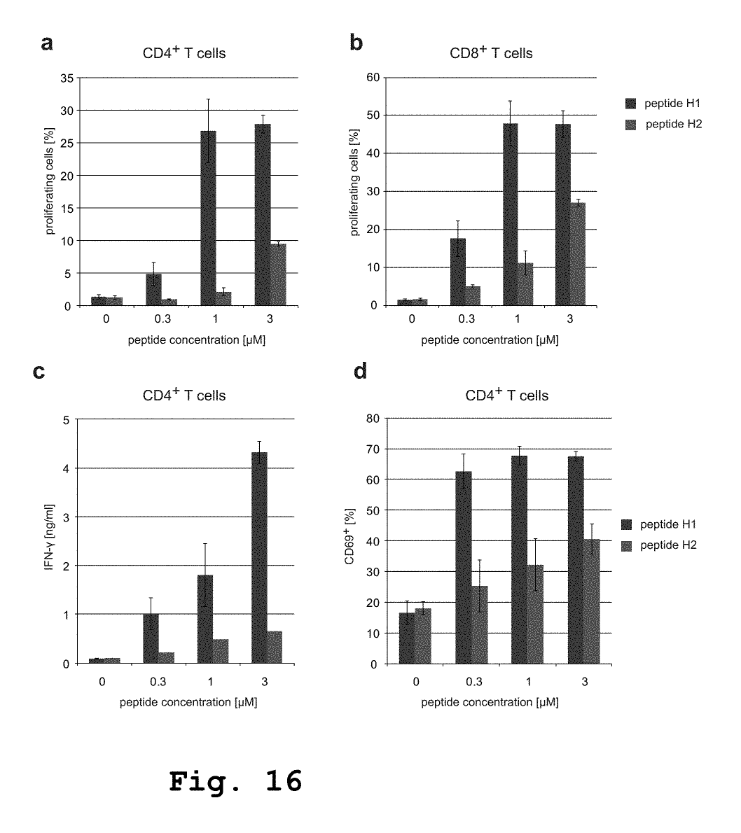

(a) Protein levels of Cbl-b in lysates prepared from mesenteric lymph nodes (mLN) or spleens of mice of the indicated genotypes as assessed with Western blotting. β-Actin is shown as loading control. (b) Representative Definiens Tissue Studio software analysis of Ly-6G stained kidney sections from FIG. 1h. (c) Cbl-b+/+ and Cbl-bΔ/Δ mice were infected intravenously with C. dubliniensis (10⁶ CFUs) and monitored for the indicated period of time. Plot depicts survival over time after infection (P values assessed with log rank test) n=5 for Cbl-b+/+ and n=6 for Cbl-bΔ/Δ cohort. (d) Cbl-b+/+ and Cbl-bΔ/Δ mice were infected intravenously with Listeria monocytogenes (10⁵ CFUs) and monitored for the indicated period of time. Plot depicts survival over time after infection (e,f) Frequency of indicated innate immune cell lineages and total immune cell counts of (e) femurs or (f) spleens of mice of the indicated genotypes as assessed by flow cytometry. n=8 for Cbl-b+/+, n=11 for Cbl-bΔ/Δ littermates. Data are shown as means±standard deviation. For panels (a,b,d,e,f) 1 representative of 3, for panel (c) 1 representative of 2 independent experiments is shown. *P<0.05, **P<0.01 as calculated with Student's t-test, unless stated otherwise.

FIG. 3: Cbl-b in immune cells protects against fungal pathology.

(a,b) Cbl-b^{+/+} mice were reconstituted with bone marrow from Cbl-b^{+/+}, Cbl-b^{Δ/Δ}, or Cbl-b^{C373A/C373A} mice after lethal irradiation. Eight weeks after reconstitution, mice were infected intravenously with Candida albicans (10⁵ CFU/21.5 g body weight) and monitored for the indicated time periods. Plots depict (a) weight loss over time after infection, as compared to starting weight (P values assessed by two-way ANOVA) or (b) survival over time after infection (P values assessed with log rank test). n=6 for all cohorts. (c,d) Cbl-b^{+/+}/Rag2^{Δ/Δ} or Cbl-b^{Δ/Δ}/Rag2^{Δ/Δ} mice were infected and scored as in (a) for (c) weight loss and (d) mortality. n=7 for Cbl-b^{+/+}/Rag2^{Δ/Δ}, n=6 for Cbl-b^{Δ/Δ}/Rag2^{Δ/Δ} mice. (e,f) Rag2^{Δ/Δ} mice were reconstituted with bone marrow from Cbl-b^{+/+}/Rag2^{Δ/Δ} or Cbl-b^{Δ/Δ}/Rag2^{Δ/Δ} mice after lethal irradiation. Eight weeks after reconstitution mice were infected and scored as in (a) for (e) weight loss and (f) mortality. n=6 for Cbl-b^{+/+}/Rag2^{Δ/Δ} into Rag2^{Δ/Δ}, n=5 for Cbl-b^{Δ/Δ}/Rag2^{Δ/Δ} into Rag2^{Δ/Δ} mice. (g,h) Cbl-b^{+/+}, Cbl-b^{Δ/Δ}, Cbl-b^{+/+}/Rag2^{Δ/Δ}, or Cbl-b^{Δ/Δ}/Rag2^{Δ/Δ} mice were infected on their shaved dorsum with 2×10⁸ CFU Candida albicans. Plot depicts fungal load in the skin 3 days after infection, plotted as CFU/total infected skin area. Dots represent individual mice. For panels (a,b,e,f,g,h) 1 representative of 3, for panels (c,d) 1 representative of 4 independent experiments is shown. *P<0.05, **P<0.01, ***P<0.001 as calculated with Student's t-test, unless stated otherwise.

FIG. 4: Immune infiltrations, CLR expression, and early fungal loads.

(a,b,c) Cellular recruitment of the indicated immune cell lineages to kidneys (a), lungs (b), and livers (c) of Cbl-b+/+ and Cbl-bΔ/Δ, mice 24 h after infection with Candida albicans (10₅ CFU/21.5 g body weight). Plots depict percentages of CD45+ hematopoietic cells (left panels) or total numbers of recruited cells per organ (right panels). n=5 for Cbl-b+/+, n=6 for Cbl-bΔ/Δ mice. (d,e) Dectin-1 protein expression on peripheral blood monocytes and neutrophils isolated from Cbl-b+/+ and Cbl-bΔ/Δ mice as assessed by blow cytometry. (d) Representative stainings and (e) Dectin-1 mean fluorescence intensities (MFI) are shown. Dots in (e) represent individual mice. (f,g) mRNA expression levels of the indicated C-type lectin receptors in (f) bone marrow cells and (g) splenocytes of Cbl-b+/+ and Cbl-bΔ/Δ mice as assessed by quantitative PCR. Experiments were done in triplicates. (h) Candida albicans fungal load in depicted organs 24 h after infection, plotted as colony forming units (CFU)/gram organ weight. Each dot represents an individual mouse. For panels (a,b,c,e,f,g,h) data are shown as means±standard deviation. For panels (a,b,c,f,g) 1 representative of 3, for panels (d,e) 1 representative of 5 independent experiments is shown. *P<0.05, **P<0.01 as calculated with Student's t-test.

FIG. 5: Cbl-b controls anti-fungal activities of dendritic cells and macrophages.

(a-e) Killing capacity of BM-neutrophils (a), BM-M (b), BM-DC (c), BM-monocytes (d), or splenic DC (e) as assessed by co-culture with Candida albicans. Assays performed in quadruplicates. (f,g) Killing capacity of BM-DC in the presence of (f) the phagocytosis inhibitor Dynasore or an anti-Dectin-1 antibody, or (g) the Syk inhibitor R406. (h,i) ROS production by intraperitoneal immune infiltrates of Cbl-b^{+/+} or Cbl-b^{Δ/Δ} mice 24 h after intraperitoneal Candida albicans infection (5*10⁶ CFU/21.5 g body weight). Plots depict (h) ROS production by immune infiltrates without re-stimulation or (i) after re-stimulation with Candida albicans. Experiments performed in triplicates. (j) Killing capacity of immune infiltrates from (h,i). (k,l) ROS production of (k) monocytes/macrophages or (l) neutrophils FACS sorted from infiltrates in (h,i). (m,n) Cbl-b^{+/+} or Cbl-b^{Δ/Δ} mice were injected with PBS liposomes or clodronate liposomes 24 h before and 24 h after intravenous infection with Candida albicans (10⁵ CFU/21.5 g body weight) and monitored for (m) weight loss, as compared to starting weight (P values assessed by two-way ANOVA), or (n) survival (P values assessed with log rank test). n=5 for PBS liposome treated, n=8 for clodronate liposome treated cohorts. For panels (a-g, j, m) data are shown as means±standard deviation. For panels (a-f) 1 representative of 5, for panels (g-n) 1 representative of 3 independent experiments is shown. *P<0.05, **P<0.01, ***P<0.001 as calculated with Student's t-test, unless stated otherwise.

FIG. 6: Phagocytosis and ROS production in response to fungal stimulation.

(a,b) Rate of phagocytosis by (a) BM-M or (b) BM-DC of the indicated genotypes co-cultured with Candida albicans. (c) Cbl-b protein expression as assessed by Western blotting from lysates prepared from C. albicans stimulated or unstimulated BM-DC, BM-M or bone marrow neutrophils derived from Cbl-b^{+/+} mice. ß-Actin blots are shown as loading control. (d-h) Reactive oxygen species (ROS) production by (d) bone marrow neutrophils, (e) BM-M, (f) BM-DC, (g) bone marrow monocytes, or (h) splenic DC co-cultured with Candida albicans and monitored in real time over the indicated time periods using the luminol assay. Experiments were performed in triplicates. Values are expressed as relative light units per 1.000 cells. (i-k) ROS production by BM-DC of the indicated genotypes stimulated with (i) C. glabrata, (j) C. krusei, or (k) C. dubliniensis, monitored and analyzed as in (d-h). Data in panels (a,b) are shown as means±standard deviation. P values for panel (a,b) were calculated with Student's t-test, for panels (d-k) with two-way ANOVA. For all experiments 1 representative of 3 independent experiments is shown. * P<0.05, **P<0.01, ***P<0.001.

FIG. 7: Cytokine release in response to fungal stimulation.

(a) Activation of Caspase-8 in BM-DC of the indicated genotypes monitored at the indicated time points after stimulation with C. albicans, using the CaspGLOW assay (P values assessed by two-way ANOVA). (b,c) Secretion of IL-1β by BM-DC of the indicated genotypes stimulated with C. albicans for 18 h in the presence of (b) an anti-Dectin-1, or isotype control antibody, or (c) the SYK inhibitor R406, or DMSO as assessed by ELISA. (d) Secretion of IL-1β by BM-M upon 18 h co-culture with C. albicans as assessed with ELISA. (e) Induction of cytokine RNA expression in BM-DC of the indicated genotypes stimulated for 2 h with C. albicans, as assessed by quantitative PCR. Experiments were done in triplicates. (f-i) Secretion of the indicated cytokines by BM-DC of the indicated genotypes upon 18 h co-culture with C. albicans in the presence of (f,g) an anti-Dectin-1, or isotype control antibody, or (h,i) the SYK inhibitor R406, or DMSO as assessed with ELISA. (j-n) Secretion of the indicated cytokines by (j,k,n) BM-DC, or (l,m) BM-M of the indicated genotypes upon 18 h co-culture with C. albicans as assessed with ELISA. Data in all panels are shown as means±standard deviation. P values for all panels except (a) were calculated with Student's t-test, for panel (a) with two-way ANOVA. For panels (a-d,f-n) 1 representative of 4, for panels (e) 1 representative of 5 independent experiments is shown. **P<0.01, *** P<0.001.

FIG. 8: Zymosan, Curdlan, LPS, CpG, and C. albicans triggered cytokine release by BM-DC and intraperitoneal inflammatory infiltrates.

(a-f) Secretion of the indicated cytokines by Cbl-b+/+, Cbl-bC373A/C373A or Cbl-bΔ/Δ BM-DC stimulated with increasing doses of either Zymosan (a-c), or Curdlan (d-f) for 18 h, as assessed by ELISA. (g-j) Secretion of the indicated cytokines by Cblb^{+/+} or Cblb^{Δ/Δ} BM-DC stimulated with the indicated doses of either LPS (g,h), or CpG (i,j) for 18 h, as assessed by ELISA. (k) Expression of the indicated cytokines in the intraperitoneal lavages of Cblb^{+/+} or Cblb^{Δ/Δ} mice 24 h after intraperitoneal infection with C. albicans as in FIG. 5h-i as assessed by ELISA. Data in all panels are shown as means±standard deviation. P values as calculated with (a-f) two-way ANOVA, or (k) Student's t-test. For all panels 1 representative of 3 independent experiments is shown. *P<0.05, **P<0.01

FIG. 9: Increased CLR mediated signaling in the absence of Cbl-b.

(a) Cbl-b^{+/+}, Cbl-b^{Δ/Δ} or Cbl-b^{C373A/C373A} BM-DC were stimulated with Candida albicans for the indicated periods of time and analyzed by Western blotting for levels of phosphorylated tyrosine. (b) BM-DC of the indicated genoytpes were stimulated as in (a) and analyzed for phosphorylated or total amounts of the indicated signaling molecules. (c) Cbl-b^{+/+} and Cbl-b^{Δ/Δ} BM-DC were stimulated for 90 min with Trehalos-6,6-dimycolate (TDM), heat killed Candida albicans (HK), or Candida albicans hyphae (Hyph), and lysates were analyzed for levels of phosphorylated Src, phosphorylated Shp-2, and phosphorylated Syk by Western blotting. β-Actin is shown as a loading control. (d) BM-DC of the indicated genoytpes were stimulated as in (a) and analyzed for phosphorylated or total amounts of the indicated molecules. (e,f) Cbl-b^{+/+} or Cbl-b^{Δ/Δ} BM-DC were stimulated with Candida albicans for 90 min or left unstimulated, fixed with 4% paraformaldehyde, stained for (e) phosphorylated Shp-2 or (f) phosphorylated Syk, and analyzed by confocal microcopy. Representative images for each genotype and time point are shown. Arrowheads depict phagosome localized (e) phosphorylated Shp-2 or (f) phosphorylated Syk. Cells were counterstained with DAPI (blue). For all panels, 1 representative of 4 independent experiments is shown.

FIG. 10: SYK and Dectin-1, -2 ubiquitination and CBL-B inhibitory peptide treatment.

(a) mRNA expression levels of the indicated C-type lectin receptors in Cblb^{+/+} and Cblb^{Δ/Δ} BM-DC assessed by qPCR. Experiments were done in triplicates. (b) Mass spectrometric analysis of the ubiquitin chains generated by in vitro ubiquitination by CBL-B. A representative MS/MS spectrum acquired for a peptide derived from K48-linked Ubiquitin chains, using a sample processed identically and in parallel to the sample shown in FIG. 11a, is shown. Both p-type (red) and γ-type (blue) ions of the selected peptide are indicated. (c,d) Immunoblot analysis of (c) Dectin-1 and (d) Dectin-2 protein expression in Cblb^{+/+} and Cblb^{Δ/Δ} BM-M upon infection with (c) C. albicans or (d) C. albicans hyphae with an anti-Dectin-1 or anti-Dectin-2 antibody, respectively. β-Actin blots are shown as loading control. (e,f) Immunoblot analysis of (e) Dectin-1 and (f) Dectin-2 ubiquitination in E64-pre-treated Cblb^{+/+} and Cblb^{Δ/Δ} BM-M after immunoprecipitation with an anti-Dectin-1 or anti-Dectin-2 antibody, respectively, with an anti-ubiquitin antibody. Dectin-1 and Dectin-2 immunoblots are shown as controls. (g) ROS production by Cblb^{+/+} and Cblb^{Δ/Δ} BM-DC co-cultured with C. albicans in the presence of the indicated concentrations of the TKB binding peptide. Plot depicts cumulative ROS production over 75 minutes for the indicated peptide concentrations. Experiments were performed in triplicates. Values are expressed as relative light units per 1.000 cells. (h,i) Cblb^{Δ/Δ} mice were infected intravenously with C. albicans (10⁵ CFU/21.5 g body weight), injected intraperitoneally with (h) 100 μg or (i) 20 μg of the TKB binding peptide at day 0 and day 2 after infection, and monitored for weight loss over time after infection as compared to starting weight (P values assessed by two-way ANOVA). n=5 for treated and untreated cohorts. (j,k) Representative kidney sections from Cblb^{+/+} mice treated as in FIG. 12g, and stained with (j) Gomori methenamine silver (GMS) or (k) for the neutrophil marker Ly-6G. Yellow arrowhead in (j) indicates fungal hyphae. n=3 for peptide treated and untreated cohorts. 4 kidney sections per mouse were analyzed. Data in (a,g) are shown as means±standard deviation. For panel (a,b) 1 representative of 4, for panel (c-k) 1 representative of 3 independent experiments is shown. ***P<0.001 as calculated with Student's t-test.

FIG. 11: Cbl-b Ubiquitinates Syk.

(a) In vitro ubiquitination of active SYK by recombinant Cbl-b, Cbl-b29-483 or C373A Cbl-b²⁹⁻⁴⁸³. Assays analyzed by Western blotting for Syk (upper panel) or Ubiquitin (lower panel). Note, that Cbl-b forms non substrate attached poly-ubiquitin chains. * denotes unspecific bands. (b) In vitro ubiquitination of inactive Syk (Syk-GST) or active Syk by Cbl-b²⁹⁻⁴⁸³. Control, no ATP. Assays were analyzed as in (a). (c) Lysates from BM-DC stimulated with Candida albicans immunoprecipitated with an anti-Cbl-b antibody or IgG control. Precipitates were probed for Cbl-b and Syk. Aliquots were blotted for Cbl-b and Syk as a loading control. (d) BM-DC were stimulated with Candida albicans in the presence or absence of MG132 (10 μM) and analyzed by Western blotting. (e) Sequence of the TKB binding peptide. Antennapedia homeodomain derived sequence and Syk derived sequences are indicated. Tyrosine 317 is shown in red, p in black circle symbolizes phosphorylation of Tyrosine 317. (f) In vitro ubiquitination of active recombinant Syk by Cbl-b²⁹⁻⁴⁸³ in the presence or absence of 100 μM TKB binding peptide. Control, no ATP. Assays were analyzed by Western blotting for Syk (upper panels) or ubiquitin (lower panels). (g) BM-DC of the indicated genotypes were stimulated with Candida albicans in the absence or presence of 100 μM TKB binding peptide and monitored for ROS production in real time, using the luminol assay. Experiments were performed in triplicates. Values are expressed as relative light units per 1000 cells (P values calculated with two-way ANOVA). (h) Killing capacity of BM-DC of the indicated genotypes as assessed by 24 h co-culture with Candida albicans in the presence or absence of 100 μM TKB binding peptide. Assays were performed in quadruplicates. Data are shown as means±standard deviation. P values were calculated with Student's t-test. For all panels, 1 representative of 3 independent experiments is shown **P<0.01, ***P<0.001.

FIG. 12: Cbl-b inhibition enhances anti-fungal immunity.

(a,b) Cbl-b^{+/+} mice were infected intravenously with Candida albicans (10⁵ CFU/21.5 g body weight), and injected intraperitoneally with 100 μg TKB binding peptide twice. Plots depict (a) weight loss (P values assessed by two-way ANOVA) and (b) survival (P values assessed with log rank test). (c) Blood urea concentration 7 days after infection. N=5 for all cohorts. (c,d,e) Cbl-b^{Δ/Δ} mice were treated as in (a-c) with 20 μg peptide. Plots depict (d) weight loss (P values assessed by two-way ANOVA; n=5 for all cohorts), (e) Blood urea concentration and (f) kidney fungal load 7 days after infection. (g,h,i) Cbl-b^{+/+} mice infected as in (a-c), were injected intraperitoneally with 100 μg of peptide twice. Plots depict (g) weight loss (P values assessed by two-way ANOVA), (h) kidney fungal load 48 h after the second peptide treatment, and (i) survival (P values assessed with log rank test). (a,d,g) Blue arrows indicate time points of peptide treatment. (j) PAS stained kidney sections from mice treated as in (g) and sacrificed 2 days after the second peptide treatment. Boxed areas shown at increased magnification. (k) Inflammatory score of infected kidneys from (j). n=3 for both cohorts. 4 sections per mouse were analyzed. (a,b,c,d,e,f,g,h,i) Data shown as means±standard deviation. Dots represent individual mice. For panels (a-c), 1 representative of 5, for panel (c-j) 1 representative of 3 independent experiments is shown. **P<0.01, ***P<0.001 as calculated with Student's t-test.

FIG. 13. CBL family TKB-domain binding peptide inhibits both CBL-B and CBL in murine DC. (a) Reactive Oxygen Species (ROS) production of dendritic cells (DC) of the indicated genotypes stably expressing the indicated shRNAs in the presence or absence of 100 μM CBL family TKB-domain containing peptide (M1) after infection with Candida albicans. Plot depicts cumulative ROS production over 90 minutes after infection. Experiments were performed in quadruplicates. Values are expressed as cumulative relative light units (RLU) per 1.000 cells. Data are shown as means±standard deviation. ***P<0.001 as calculated with Student's t-test.

FIG. 14. CBL family TKB-domain binding peptide treatment enhances the proliferative response of murine CD4⁺ T cells. CD4⁺ T cells MACS purified from spleens and pooled lymph nodes of cblb^{+/+} (a,b) or cblb^{Δ/Δ} mice (c,d) were labeled with cell proliferation dye and stimulated with the indicated concentrations of plate bound CD3 and soluble CD28 antibodies or only plate bound CD3 antibodies in the absence or presence of the indicated concentrations of CBL family TKB-domain binding peptides (M3) (a,c) or scrambled control peptides (M4) (b,d) for 3 days. Histogram overlays depict the proliferation dependent dilution of cell proliferation dye in response to T cell receptor stimulation in the presence or absence of the indicated concentrations of peptides after 3 days of stimulation.

FIG. 15. CBL family TKB-domain binding peptide treatment enhances the in vivo anti-tumor immune response in a melanoma model. (a,b) Three cohorts of C57BL/6 mice (n=10 per cohort) were inoculated with 10⁶ B16-SIY tumor cells subcutaneously, treated with vehicle, peptide M1, or peptide M2 every other day and monitored for tumor volume (a) and bodyweight (b) over time. Blue arrows indicate time points of peptide treatment. Data are shown as means±SEM. Statistical significance was assessed using two-way ANOVA. *P<0.05, **P<0.01.

FIG. 16. CBL family TKB-domain binding peptide treatment enhances the proliferation, cytokine production, and activation of human T cells. Human CD4⁺, or CD8⁺ T cells were purified from PBMC, labeled with the cell proliferation dye CFSE and stimulated with plate bound CD3/CD28 antibodies for 3 days. (a,b) Graphs depict the proliferative response of CD4⁺ (a) or CD8⁺ (b) T cells in response to CD3/CD28 stimulation in the presence of the indicated concentrations of peptides H1 or H2, as assessed by quantifying the proportion of cells with diluted CFSE content. (c) Supernatants from cells treated as in (a,b) were assayed for IFN-γ levels using ELISA. (d) Cells treated as in (a,b) were stained for the activation marker CD69. The graph depicts the percentage of CD69⁺ cells as a fraction of total CD4⁺ cells.

### EXAMPLES

### Example 1—Materials & Methods

Mice: Cbl-b^{Δ/Δ}, Cbl-b^{C373A/C373A} and Rag2^{Δ/Δ} deficient mice have been described previously and were bred on a C57BI/6 background. Mouse genotypes were assessed by PCR. Of note, only age- and sex-matched littermates from respective breedings were used for experiments. All mice were bred and maintained in accordance with ethical animal license protocols complying with the current Austrian law.

Generation of Bone Marrow Chimeric Mice:

To generate bone marrow chimeric mice, 8-week-old wild type or Rag2^{Δ/Δ} deficient C57BL6 mice were split dose irradiated (2×6 Gy) and reconstituted 18 h after the second irradiation with 3 million donor cells by intravenous injection. Experiments were carried out 8 weeks later to allow for full reconstitution. Reconstitution efficiency under the applied conditions was tested by using congenic CD45.1 recipient mice and staining of splenocytes for CD45.1 (recipient derived) and CD45.2 (donor derived), 8 weeks after reconstitution, and was routinely >98%.

Isolation and differentiation of neutrophils, monocytes, and dendritic cells: Neutrophils were isolated from tibias and femurs of mice using a Percoll-gradient (GE Healthcare) as previously described (Boxio et al., 2004) or were purified using MACS technology (Miltenyi) after Ly-6G labeling. Bone marrow monocytes were purified by FACS sorting after labeling with antibodies specific for CD11b, Ly-6G, and Ly-6C. Splenic dendritic cells were FACS sorted after labeling with antibodies to CD19, TCRβ, Ly-6G, and CD11c. Purified neutrophils, monocytes, and splenic dendritic cells were cultured in RPMI-1640 medium (PAA). BM-DC or BM-M were obtained by differentiation from bone marrow precursor cells. Peripheral blood monocytes or neutrophils were obtained by sub-mandibular bleeding and collected in potassium EDTA tubes (Sarstedt, Germany). Red blood cells were lysed using an Ammonium-Chloride-Potassium (ACK) buffer.

Flow Cytometry:

Antibody labeling of leukocytes was carried out in FACS staining buffer (PBS supplemented with 2% FBS and 2 mM EDTA) on ice for 30 min after blocking of Fc-receptors. Cell viability was assessed with the Fixable Viability Dye eFluor780 (eBioscience). Immune cell recruitment was assessed by Collagenase 4 (Worthington)/DNAse (Roche) digestion of organs, viability staining, Fc-blocking, antibody staining, and subsequent flow cytometric analysis. Samples were acquired with a BD LSRII flow cytometer and data were analyzed using FlowJo software (Treestar).

Measurements of Cytokine Release, ROS Production, Caspase-8 Activity:

Cytokine secretion by cell culture supernatants or in the peritoneal lavage was assessed with ELISA kits purchased from eBioscience (IL-1β, TNF, IL-6, IL-12, IL-23) or R&D Systems (IL-1Ra) according to the manufacturer's instructions. Production of reactive oxygen species (ROS) was measured with as assay using luminol as the probe in real-time over 90 min as described previously (Bourgeois et al., 2009). Caspase-8 activity was assessed using the CaspGLOW assay (Promega) according to the manufacturer's instructions.

Phagocytosis Assay:

For phagocytosis assays Candida albicans (strain SC5314) were Alexa 488 labeled in 100 mM HEPES buffer (pH 7.5). BM-DC or BM-M were co-cultured with labeled Candida albicans at 37° C. for 45 minutes. Fluorescence by fungal cells adherent to, but not phagocytosed by, phagocytes, were quenched with trypan blue (Sigma Aldrich), and the rate of phagocytosis was assessed by flow cytometry.

Killing Assay:

To assess the fungicidal activity, the indicated immune cells were plated in replicates at a density of 1×10⁵ cells/well in 96-well plates. Cells were incubated with Candida albicans at a multiplicity of infection of 1:500 for 24 h. After incubation, phagocytes were fixed by the addition of paraformaldehyde at a final concentration of 4%. Subsequently, Candida albicans were stained with Crystal Violet (Sigma) and killing was assessed by a comparison of colony numbers in wells with or without phagocytes.

Mouse Infection and Clodronate Liposome Treatment:

8-10 week old mice were infected with 10⁵ colony forming units (CFU) of C. albicans (strain SC5314), 10⁵ colony forming units (CFU) of C. dubliniensis, or 10⁵ CFUs of Listeria monocytogenes (strain EGD) intravenously. C. albicans CFUs were assessed from various organs as described (Wirnsberger et al., 2014). For clodronate or PBS liposome treatment experiments, mice were injected intraperitoneally with 100 ul/10 g mouse weight of liposomes both 24 h before and 24 h after intravenous C. albicans infections. Clodronate or PBS control liposomes were obtained from clodronateliposomes.org, and handled according to the manufacturer's instructions. All animal infection experiments were evaluated by the ethics committee of the Medical University of Vienna and approved by the Federal Ministry of Science and Research, Vienna, Austria.

BM-M and BM-DC Stimulation Experiments:

BM-M and BM-DC were stimulated with C. albicans (strain SC5314), Zymosan (Invivogen), Curdlan (Invivogen), TDM (Invivogen), CpG (Invivogen), LPS (Invivogen), or C. albicans hyphae. For inhibition experiments cells were treated with the Syk inhibitor R406 (Selleckchem), anti-Dectin-1 antibody (Invivogen), MG132 (Calbiochem), or Dynasore (Sigma-Aldrich), 30 min prior to stimulation (MOI: Immune cell: C. albicans=1:2). Fungal isolates of Candida krusei, Candida glabrata, and Candida dubliniensis were kindly provided by the Allgemeines Krankenhaus Vienna (AKH). Fungal isoates were tested by PCR to verify the respective species.

Quantitative PCR:

mRNA was isolated using the SV-Total RNA Isolation System (Promega) or Rneasy Kit (Qiagen), reverse transcribed using the Reverse Transcription System (Promega) or the iScript cDNA synthesis Kit (Biorad). QPCR was performed with KAPA SYBR Fast Universal (Peqlab).

Western Blotting and Immunoprecipitation:

Western blotting was performed according to standard protocols. Blots were blocked for 1 hour with 5% bovine serum albumin in 1×TBS/0.1% Tween-20, or 4% BSA in 1×TBS/0.1% Tween-20 for phosphorylated epitopes, followed by overnight incubation at 4° C. with primary antibodies. Subsequently, blots were washed three times in 1×TBS/0.1% Tween-20 for 15 min, followed by incubation with HRP-conjugated secondary antibodies (1:2500; GE Healthcare # NA9340V) for 45 min at room temperature, washed three times in 1×TBS/0.1% Tween-20 for 15 min and visualized using enhanced chemiluminescence (ECL Plus, Pierce). Anti-β-Actin mAbs were used to control for protein loading. For Cbl-b-immunoprecipitation experiments, cells were stimulated with C. albicans, lysed in RIPA buffer (Cell Signaling Technology) containing phosphatase and proteinase inhibitors (Halt Protease and Phosphatase inhibitor Cocktail; Thermo Scientific), pre-cleared with Protein A/G Plus Agarose beads (Santa Cruz Biotechnology), and incubated overnight with an anti-Cbl-b antibody. Precipitates were analyzed by Western blotting using the indicated antibodies. For Dectin-1 and Dectin-2 immunoprecipitates, BMM of the indicated genotypes were pre-treated with E-64 (10 μM) for 30 min, infected with C. albicans yeast or hyphae for the indicated periods of time, lysed in RIPA buffer containing 2% SDS on ice for 30 min, and diluted with RIPA buffer without SDS for a final SDS concentration of 0.4%. After brief sonication, lysates were immunoprecipitated with an anti-Dectin-1 aor anti-Dectin-2 antibody at a concentration of 1 μg/ml. Precipitates were analyzed by Western blotting with the indicated antibodies.

In vitro ubiquitination assays: Reaction mixtures for in vitro ubiquitination assays contained 100 nm Ubel (purified in-house), 500 Nm UbcH5b (Enzo Life Sciences), 150 nm full length human Cbl-b (Abnova) or human Cbl-b²⁹⁻⁴⁸³, or a catalytically inactive human C373A Cbl-b29-483 mutant, and 100 mM ubiquitin (Sigma-Aldich) with 300 nm of substrate in reaction buffer (20 mM Tris-Hcl pH 7.5, 150 mM NaCl, 10 mM MgCl₂ and 1 mM DTT). Substrates were active (phosphorylated) human Syk (Merck Millipore) and inactive (non-phosphorylated) human Syk-GST (Life technologies). 1 MM ATP was added to initiate the reaction and incubated at 37° C. for 30 min. For TKB binding peptide mediated Cbl-b inhibition, Cbl-b was pre-incubated with the TKB binding peptide at a concentration of 100 μM for 20 min at 24° C., before addition to the reaction mixture. Assays were resolved by SDS-PAGE, transferred on a nitrocellulose membrane (GE Healthcare) and blotted with an anti-ubiquitin antibody, or an anti-Syk antibody.

Production of Recombinant Cbl-b²⁹⁻⁴⁸³ and C373A Cbl-b²⁹⁻⁴⁸³:

Cbl-b²⁹⁻⁴⁸³ and C373A Cbl-b10²⁹⁻⁴⁸³ coding sequences were cloned into the pGEX-2TK vector to obtain Gluthathione-S-transferase (GST) fusion proteins. Expression in Escherichia coli BL21 was induced with 0.3 mM isopropyl-D-thiogalactopyranoside at OD600 of 1.0 in Terrific Broth medium, and incubated overnight at 20° C. Cells were lysed in lysis buffer (20 mM Tris-Hcl pH 7.5, 100 mM NaCl, 1 mM EDTA, 0.5% Triton X-100), sonicated briefly three times, and incubated overnight with Glutathione Sepharose 4B (GE Healthcare) at 4° C. Subsequently, Sepharose beads were washed 5 times with lysis buffer, and bound proteins were eluted with elution buffer (50 mM Tris-Hcl, pH 8.0, and 20 mM reduced glutathione). Eluates were tested for size and purity with SDS-PAGE and Western blotting.

Histopathology:

For histopathology analyses, kidneys were fixed in 10% neutral buffered formalin, processed according to standard procedures, embedded in paraffin, and sectioned. 2 μm thick sections were stained with haematoxylin and eosin (H&E), periodic-acid-Schiff (PAS), or anti-Ly-6G (Biolegend; clone 1A8). Immunohistochemistry was performed with a primary antibody dilution of 1:100 with DAB enhancement after antigen retrieval (BOND Epitope Retrieval kit, Leica). 4 μm sections were stained with Gomori Methenamine Silver (GMS; Merck). Stained slides were scanned using a Pannoramic slide scanner (3D Histech) and evaluated for severity of inflammation and intra-lesional fungal burden based on the following criteria:

| | | INTRALESIONAL |
|---|---|---|
| | RENAL INFLAMMATION | FUNGAL BURDEN |
| | (Based on H&E and PAS) | (Based on PAS and GMS) |
| | Proportion of renal | Extent of presence of intact |
| | parenchyma and/or pelvis | fungal bodies (hyphae, |
| | involved by | pseudohyphae or spores) |
| | tubulointerstitial nephritis | within and around foci of |
| SCORE | and/or pyelonephritis | inflammation |
| 0 | None/Not significant | None/Not significant |
| 1 | Less than 10% | Scant presence in less than |
| | | 10% of inflammatory foci |
| 2 | 10% to 25% | Mild to moderate presence in |
| | | 10% to 25% of inflammatory |
| | | foci |
| 3 | 25% to 50% | Moderate to significant |
| | | presence in 25% to 50% of |
| | | inflammatory foci |
| 4 | Greater than 50% | Significant presence in |
| | | greater than 50% of |
| | | inflammatory foci |

Definiens Tissue Studio™ software was applied to evaluate the extent of infiltration by Ly-6G⁺ cells in affected kidneys. Regions and cells of interest were manually classified in representative areas of slides to train the software algorithm to identify Ly-6G positive target objects and exclude non-target objects. Target objects were identified on the basis of morphology and positive immunohistochemical staining. The optimized algorithm was then applied to analyze all slides automatically. The validity of the analysis was confirmed by histopathologic verification of representative slides from both groups.

Peptide Synthesis:

Solid phase peptide synthesis (SPPS) was used for TKB binding peptide synthesis on an ABI 433a Peptide Synthesizer (Applied-Biosystems). Peptides were purified with an Ultimate 3000 RP-HPLC system (Thermo Scientific), and analyzed with a 4800 MALDI TOF/TOF analyzer (ABSciex). Lyophilized peptides were resolved in PBS and stored at −80° C.

Immunofluorescence:

BM-CD were seeded on μ-slides (IBIDI) 24 h before infection with C. albicans. After infection cells were fixed in 4% PFA on ice for 20 min, permeabilized (0.2% Triton X-100 in PBS) for 10 min at room temperature, and blocked (3% BSA in PBS) for 1 hour at room temperature. Subsequently, cells were incubated with primary antibodies against Syk (1:100) or Shp-2 (1:100) in 3% BSA at overnight. Cells were then washed in PBS and incubated with an anti-rabbit-Alexa555 antibody (1:500) in 3% BSA at room temperature for 1 h. After washing in PBS cells were counterstained with DAPI, and directly imaged with a Zeiss LSM780 Confocal microscope.

NanoLC-MS Analysis:

The nano HPLC system used for this study was an UltiMate 3000 HPLC RSLC nano system (Thermo Scientific) coupled to a Q Exactive Plus mass spectrometer (Thermo Scientific), equipped with a Proxeon nanospray source (Thermo Scientific). Peptides were loaded onto a trap column (Thermo Fisher Scientific, Amsterdam, Netherlands, PepMap C18, 5 mm×300 μm ID, 5 μm particles, 100 A pore size) at a flow rate of 25 μL min⁻¹ using 0.1% TFA as mobile phase. After 10 min the trap column was switched in line with the analytical column (Thermo Fisher Scientific, Amsterdam, Netherlands, PepMap C18, 500 mm×75 μm ID, 2 μm, 100 Å). Peptides were eluted using a flow rate of 230 nl min′, and a binary 2 h gradient for 165 min. The gradient was started with 98% water/formic acid (99.9/0.1; v/v) and 2% water/acetonitrile/formic acid (19.92/80/0.08; v/v/v), and increased to 35% water/formic acid (99.9/0.1; v/v) and 65% water/acetonitrile/formic acid (19.92/80/0.08; v/v/v) over 120 min, followed by a gradient to 10% water/formic acid (99.9/0.1; v/v) and 90% water/acetonitrile/formic acid (19.92/80/0.08; v/v/v) over 5 min, kept at this ratio for 5 min and subsequently decreased over 2 min back to 98% water/formic acid (99.9/0.1; v/v) and 2% water/acetonitrile/formic acid (19.92/80/0.08; v/v/v) for equilibration at 30° C. The Q Exactive mass spectrometer was operated in data-dependent mode, using a full scan (m/z range 370-1650, nominal resolution of 70 000, target value 3E6) followed by MS/MS scans of the 12 most abundant ions. MS/MS spectra were acquired using normalized collision energy 27%, isolation width of 2 and the target value 1E5. Precursor ions selected for fragmentation (charge state 2 and higher) were put on a dynamic exclusion list for 10 sec. The underfill ratio was set to 20% resulting in an intensity threshold of 4E4. The peptide match feature and the exclude isotopes feature were enabled.

Mass Spectrometry Data Analysis:

For peptide identification the RAW-files were loaded into Proteome Discoverer (version 1.4.0.288, Thermo Scientific). All created MS/MS spectra were searched using the search engine node MSAmanda (Dorfer et al., 2014) against the flybase sequence database (22,256 sequences; 20,222,850 residues) and also against the Swissprot sequence database, using the taxonomy human (20,170 sequences, 11,318,213 residues). The following search parameters were used: Beta-methylthiolation on cysteine was set as a fixed modification, oxidation on methionine, acetylation on lysine, phosphorylation on serine, threonine and tyrosine, deamidation on asparagine and glutamine, and ubiquitinylation on lysine were set as variable modifications. Monoisotopic masses were searched within unrestricted protein masses for tryptic peptides. The peptide mass tolerance was set to ±5 ppm and the fragment mass tolerance to ±0.03 Da. The maximal number of missed cleavages was set to 2. The result was filtered to 1% FDR using Percolator algorithm integrated in Proteome Discoverer. The localization of the sites of variable modifications within the peptides was performed with the tool ptmRS, integrated in Proteome Discoverer and based on phosphoRS (Taus et al., 2011).

Data Analysis and Statistics.

All values in the paper are given as means±standard deviation unless stated otherwise. All in vitro experiments were reproduced 3-5 independent times. In vivo experiments were reproduced 2-6 times. Figures and statistical analyses were generated using PraphPad Prism software (GraphPad Software). No mice were excluded from the analyses and histopathologic analyses using Definies Tissue studio software were performed blindly. Mice were allocated to experimental groups based upon their genotypes and randomized within their sex- and age matched groups. Since mice were inbred and sex- and age-matched, similar variance between experimental cohorts was assumed. An a priori sample size estimation was not performed. Group sizes were based upon the empirically assessed variability of the model systems or assays used, and groups contained as many mice as possible to minimize type I and type II errors. Data were analyzed using the unpaired two-tailed Student's t-test, or two way ANOVA, as indicated. For survival analyses, log rank tests were performed. P values <0.05 were accepted as statistically significant. The following antibodies were used in this study:

| Antigen | Clone | Distributor |
|---|---|---|
| Syk/SYK | D115Q | Cell Signaling Technologies |
| phospho Syk | C87C1 | Cell Signaling Technologies |
| Src | 36D10 | Cell Signaling Technologies |
| phospho Src | D7F2Q | Cell Signaling Technologies |
| Plc-γ2 | Q-20 | Santa Cruz Biotechnology |
| phospho-Plc-γ2 | Tyr1217 | Cell Signaling Technologies |
| NF-κB p65 | E498 | Cell Signaling Technologies |
| phospho-NF-κB p65 | 93H1 | Cell Signaling Technologies |
| Shp-2 | C-18 | Santa Cruz Biotechnology |
| phospho-Shp-2 | Tyr542 | Cell Signaling Technologies |
| β-Actin | 13 E 6 | Cell Signaling Technologies |
| Ubiquitin | P4D1 | Santa Cruz Biotechnology |
| Ly-6G FITC | 1A8 | BD Biosciences |
| CD8 Brilliant Violet 605 | 53-6.7 | Biolegend |
| TCRβ Brilliant violet 421 | H57-597 | BD Biosciences |
| NK-1.1 APC | PK136 | BD Biosciences |
| CD19 PE | 6D5 | BD Biosciences |
| CD11c FITC | HL3 | Biolegend |
| CD16/32 purified | 2.4G2 | BD Biosciences |
| Ly-6C PerCP-Cy5.5 | AL-21 | BD Biosciences |
| anti-rabbit Alexa555 | polyclonal | Invitrogen |

The following PCR primers were used in this study:

| | | | amplicon |
|---|---|---|---|
| Target | fwd | rev | size |
| Clec4n | CTGCCCAAATCACTGGAAGT (SEQ | GAAATTCTGCTCCGCTTCAG (SEQ | 142 bp |
| | ID NO: 18) | ID NO: 19) | |
| Clec7a | GAACCACAAGCCCACAGAAT (SEQ | TAGGAAGGCAAGGCTGAGAA (SEQ | 95 bp |
| | ID NO: 20) | ID NO: 21) | |
| Clec4d | TACGCTGGACGAGAGGAAGT (SEQ | ACAGGACAGCAGGTCCAAGT (SEQ | 112 bp |
| | ID NO: 22) | ID NO: 23) | |
| Clec4e | CAAGTGCTCTCCTGGACGAT (SEQ | CTGTAAGTTCTGCCCGGAAA (SEQ | 114 bp |
| | ID NO: 24) | ID NO: 25) | |
| TNF | CAAAATTCGAGTGACAAGCCTG | GAGATCCATGCCGTTGGC (SEQ ID | 88 bp |
| | (SEQ ID NO: 26) | NO: 27) | |
| IL-6 | GAGGATACCACTCCCAACAGACC | AAGTGCATCATCGTTGTTCATACA | 140 bp |
| | (SEQ ID NO: 28) | (SEQ ID NO: 29) | |
| IL-12 | GGAAGCACGGCAGCAGAATA (SEQ | AACTTGAGGGAGAAGTAGGAATGG | 159 bp |
| | ID NO: 30) | (SEQ ID NO: 31) | |
| IL-23 | GCAGATTCCAAGCCTCAGTC (SEQ | TTCAACATATGCAGGTCCCA (SEQ | 86 bp |
| | ID NO: 32) | ID NO: 33) | |
| IL-1β | CAACCAACAAGTGATATTCTCCATG | GATCCACACTCTCCAGCTGCA (SEQ | 120 bp |
| | (SEQ ID NO: 34) | ID NO: 35) | |

Human T Cell Assays:

Peripheral blood mononuclear cells (PBMC) were purified from human blood using a Lymphoprep™ PBMC isolation kit (Stemcell Technologies), according to the manufacturer's instructions. Human CD4+ or CD8+ T cells were purified from PBMC using CD4 or CD8 MicroBeads (Miltenyi Biotec), respectively, and MACS technology (Miltenyi Biotec). For proliferation assays, flat-bottom 96-well plates were coated with CD3 (OKT3, 0.1 μg/ml) and CD28 (CD28.2, 0.1 μg/ml) antibodies in PBS at 37 degrees C. for 2 h. To monitor proliferative expansion, assays were carried out with carboxyfluorescein succinimidyl ester (CFSE; eBioscience) labeled T cells. T cells were cultured in X-VIVO 15 chemically defined, serum free hematopoietic cell medium (Lonza), with the indicated concentrations of peptide or vehicle control. After 72 h days, cells were labeled with antibodies against CD4 (RPA-T4, Brilliant Violet 510 labeled, Biolegend) or CD8 (RPA-T8, APC-eFluor 780 labeled, eBioscience), and CD69 (FN50, PE/Cy7 labeled, Biolegend), and analyzed on a Beckman Coulter Gallios flow cytometer. Data were analyzed using Beckman Coulter Kaluza software. IFN-γ secretion into cell culture supernatants of the proliferation assays was determined by enzyme-linked immunosorbent assays (ELISA) according to the manufacturer's (BD Biosciences) instructions.

Murine T Cell Assays:

Murine CD4+ T cells were purified from pooled spleens and lymph nodes using MACS technology (Miltenyi Biotec). For proliferation assays, flat-bottom 96-well plates were coated with CD3 (145-2C11, Biolegend) and CD28 (37.51, Biolegend) antibodies. Purified T cells were labeled with the cell proliferation dye eFluor 670 (eBioscience) and cultured in IMDM cell culture medium (Gibco), supplemented with 10% fetal bovine serum (Sigma), L-Glutamine (Thermo Fisher), Penicillin and Streptomycin (Thermo Fisher), in the presence of 5 ng/ml of recombinant IL-2 (Biolegend) and the indicated concentrations of peptide. After 3 days, cells were stained with the fixable viability dye eFluor 780 (Thermo Fisher), and stained with a CD4 antibody (RM4-5, Brilliant Violet 711, Biolegend). Cells were analyzed with a BD LSR Fortessa II flow cytometer (Becton Dickinson), subsequently, and data were analyzed using the FlowJo v10.0.8 software (Tree Star).

B16-SIY In Vivo Tumor Model:

B16-SIY tumor cells as described in (Spranger, 2014) were cultured in DMEM cell culture media (Gibco) supplemented with 10% fetal bovine serum (Sigma), L-Glutamine (Thermo Fisher), Penicillin and Streptomycin (Thermo Fisher). 10⁶ tumor cells were transplanted intradermally into C57Bl/6 mice. Mice were injected intraperitoneally with 100 μg of the indicated peptides or vehicle control every other day starting 24 h after tumor cell inoculation. Bodyweight and tumor volume were measured at the indicated time points, subsequently.

Generation of Murine Hoxb8-Hematopoietic Progenitors and shRNA Mediated Cbl Knock-Down:

Flt3-Hoxb8-hematopoietic progenitors were generated, cultured, and differentiated into GM-CSF dendritic cells (DC) as previously described (Redecke, 2013) from total cblb^{+/+} or cblb^{Δ/Δ} bone marrow cells. Lentiviral vectors containing the MiR-E backbone for the generation of stable Hoxb8-hematopoietic progenitor shRNA lines targeting cbl or renilla luciferase were described previously (Fellmann, 2013). ShRNAs targeting cbl or renilla luciferase were cloned into these lentiviral vectors by PCR amplification from 97mer oligonucleotides (cb1:5′-TGCTGTTGACAGTGAGCGCTACGACGATGATGATGATGAATAGTGAAGCCACAGATGTATTCATCATCAT CATCGTCGTAATGCCTACTGCCTCGGA-3′ (SEQ ID NO: 37); renilla luciferase: 5f-TGCTGTTGACAGTGAGCGCAGGAATTATAATGCTTATCTATAGTGAAGCCACAGATGTATAGATAAGCAT TATAATTCCTATGCCTACTGCCTCGGA-3′ (SEQ ID NO: 38); IDT) using mir-E fwd (5′-AGAAGGCTCGAGAAGGTATATTGC-3′ (SEQ ID NO: 39)), or mir-E rev (5′-GCTCGAATTCTAGCCCCTTGAAGTCCGAGG-3′ (SEQ ID NO: 40)) primers as described previously (Fellmann et al., 2013). Cell lines stably expressing cbl or control shRNAs were obtained by lentiviral transduction and FACS sorting of infected cell lines based upon eGFP reporter gene expression. ShRNA expressing cell lines were differentiated into dendritic cells (DC) in the presence of GM-CSF and assayed for ROS production as described above.

### Peptide Sequences Used for Murine or Human Experiments:

| | | |
|---|---|---|
| M1 | DRQIKIWFQNRRMKWKKSTVSFNPYEPTGGPWG | antennapedia CPP-CBL- |
| | * | family TKB domain |
| | (SEQ ID NO: 41) | binding peptide |
| | | (*phosphorylated |
| | | tyrosine) |
| M2 | DRQIKIWFQNRRMKWKKSTPNFGVYGTSPWGPE | antennapedia CPP- |
| | * | scrambled peptide |
| | (SEQ ID NO: 42) | (*phosphorylated |
| | | tyrosine) |
| M3 | KIKKTKKKGRKKIKKTKKKGRKSTVSFNPYEPTGGPWG | dNP2 CPP-CBL-family |
| | * | TKB domain binding |
| | (SEQ ID NO: 43) | peptide |
| | | (*phosphorylated |
| | | tyrosine) |
| M4 | KIKKTKKKGRKKIKKTKKKGRKSTPNFGVYGTSPWGPE | dNP2 CPP-scrambled |
| | * | peptide |
| | (SEQ ID NO: 44) | (*phosphorylated |
| | | tyrosine) |
| H1 | DRQIKIWFQNRRMKWKKSTVSFNPYEPELAPWA | antennapedia CPP-CBL- |
| | * | family TKB domain |
| | (SEQ ID NO: 45) | binding peptide |
| | | (*phosphorylated |
| | | tyrosine) |
| H2 | DRQIKIWFQNRRMKWKKEAVPLYAWYPNPSTFES | antennapedia CPP- |
| | * | scrambled peptide |
| | (SEQ ID NO: 46) | (*phosphorylated |
| | | tyrosine) |

The table above lists additional peptides used in this study. The first column depicts the name and species origin (M for mouse, H for human) of SYK derived peptide sequences in single letter amino acid code. Bold sequences in the second column depict amino acid sequences of cell permeable peptides (CPP) in single letter code. Shown with asterisk (*) in the second column is the phosphorylated central tyrosine of SYK derived or scrambled peptide sequences. Scrambled peptides were derived by scrambling the amino acid sequence of either mouse or human SYK derived sequences around the central phospho-tyrosine, but leaving CPP moieties intact.

### Example 2—Loss of Cbl-b or Cbl-b Catalytic Activity Ameliorates Fungal Pathology

To test for a potential role of Cbl-b in anti-fungal immunity, Cbl-b^{Δ/Δ} and control Cbl-b^{+/+} littermates were systemically challenged with a lethal dose of Candida albicans. In addition, Cbl-b^{C373A/C373A} mice, carrying catalytically inactive Cbl-b (Paolino et al., 2011), were infected with Candida albicans. Loss of Cbl-b protein (FIG. 2a) or Cbl-b catalytic function markedly ameliorated fungal pathology as shown by decreased weight loss after infection (FIG. 1a) and by significantly delayed mortality (FIG. 1b) when compared to Cbl-b^{+/+} littermate animals. In line with these results, significantly reduced fungal burdens in kidneys, spleens, livers, and lungs of Cbl-b^{Δ/Δ} mice could also be observed, when compared to their Cbl-b^{+/+} littermate controls (FIG. 1c). Histopathologic analysis of affected kidneys 7 days after systemic infection confirmed these results; Cbl-b^{Δ/Δ} mice manifested reduced renal inflammation, as assessed by Periodic-acid-Schiff staining (FIG. 1d,e) and reduced numbers of Candida albicans yeasts and hyphae (FIG. 1f,g), as assessed with Gomori Methenamine Silver (GMS) staining. In line with a decreased fungal burden, a significant reduction in neutrophil infiltration was detected, as assessed by staining for the neutrophil specific marker Ly-6G on kidney sections of infected Cbl-b^{Δ/Δ} mice when compared to Cbl-b^{+/+} littermate controls (FIG. 1h,i). The protective effect of Cbl-b deficiency was not limited to infections with Candida albicans, since systemic infection with a lethal dose of a clinical isolate of Candida dubliniensis yielded similar results with respect to mortality rates and kinetics, when Cbl-b^{Δ/Δ} mice were compared to Cbl-b^{+/+} littermate controls (FIG. 2c). To assess, whether the protective effect of Cbl-b deficiency in severe fungal infection was the result of a broad deregulation of innate immune function, Cbl-b deficient and littermate control mice were infected with the gram-positive bacterium Listeria monocytogenes. Loss of Cbl-b did not affect the mortality of Listeria monocytogenes infected mice (FIG. 2d). The results show that loss of Cbl-b results in marked resistance to systemic Candida albicans and Candida dubliniensis infections.

To gain insight into the cellular basis of the observed protective effect, bone marrow chimeric animals were generated by lethally irradiating C57Bl/6 recipient mice and reconstituting them with bone marrow from syngeneic Cbl-b^{C373A/C373A} or Cbl-b^{+/+} littermate mice. After reconstitution, the cohorts were infected with a lethal dose of Candida albicans. Loss of Cbl-b in the radiosensitive hematopoietic compartment mirrored full body Cbl-b deficiency (FIG. 3a,b). Due to the role of Cbl-b in adaptive immunity, it was next tested for a potential contribution of adaptive immune cells to the observed anti-fungal activity. To this end, Cbl-b^{+/+}/Rag2^{Δ/Δ} and Cbl-b^{Δ/Δ}/Rag2^{Δ/Δ} double deficient animals that lack an adaptive immune system were generated. Importantly, Cbl-b deficiency in the innate immune system, and in the absence of adaptive immunity, closely resembled the full body knock-out phenotype upon lethal infection with Candida albicans (FIG. 3c,d). Additionally, bone marrow chimeric animals were generated by lethal irradiation of Rag2^{Δ/Δ} recipients and reconstitution with Cbl-b^{Δ/Δ}/Rag2^{Δ/Δ} or Cbl-b^{+/+}/Rag2^{Δ/Δ} bone marrow grafts, to restrict potential effects of Cbl-b deficiency to the innate immune system. Again it could be observed that loss of Cbl-b confers a protective effect to systemic infection with Candida albicans (FIG. 3e,f), indicating that Cbl-b controls innate anti-fungal immunity.

To test for potential effects of Cbl-b deficiency on the development or maintenance of innate immune cells relevant for the anti-fungal defense, cellularity and cellular composition of the main innate immune lineages in the bone marrow and spleen of Cbl-b^{Δ/Δ} and Cbl-b^{+/+} littermates were assessed. Slightly elevated levels of both bone marrow and splenic Ly-6G⁺ neutrophils and splenic NK cells could be observed (FIG. 2e,f). To address whether this might affect the infiltration of monocytes or neutrophils, their recruitment in response to systemic Candida albicans challenge 24 h after infection was quantified. At this early time point after infection, comparable, or even slightly decreased immune infiltration into kidneys, lungs, and livers of the Cbl-b^{Δ/Δ} mice could be observed (FIG. 4a,b,c). Recognition of Candida albicans cell wall β-glucan by the CLR family member Dectin-1 has been shown to be of crucial importance in the anti-fungal immune response (Brown et al., 2001). Comparable levels of Dectin-1 expression on peripheral blood monocytes and neutrophils of Cbl-b^{+/+} and control Cbl-b^{+/+} littermate mice were detected (FIG. 4d,e). Moreover, mRNA expression of Dectin-1 (Clec7a), as well as Dectin-2 (Clec4n), and Dectin-3 (Clec4d) as well as Mincle (Clec4e) were comparable in splenocytes and bone marrow cells of Cbl-b^{Δ/Δ} and Cbl-b^{+/+} animals, as assessed by qPCR (FIG. 4f,g). Importantly, although Cbl-b^{Δ/Δ} and Cbl-b^{+/+} animals exhibited comparable early cellular recruitment patterns in response to systemic Candida albicans challenge, fungal burdens in affected organs were already significantly decreased 24 hours after infection (FIG. 4h), supporting the notion that loss of Cbl-b results in an increased fungicidal activity of innate immune cells.

### Example 3—Loss of Cbl-b Confers Resistance to Both Systemic and Cutaneous Fungal Infections

To test whether the protective effect of Cbl-b deficiency could be extended to other routes of infection, a model of cutaneous Candida albicans infection was used (Igyarto et al., 2011). In this model an increased resistance of Cbl-b^{Δ/Δ} mice to skin colonization by Candida albicans was again observed, when compared to littermate controls, as evidenced by a significantly decreased fungal burden (FIG. 3g). Since immune reactions against cutaneous infection with Candida albicans are characterized by a crosstalk between adaptive and innate immune cells (Igyarto et al., 2011; Kashem et al., 2015), Candida albicans skin infections were performed on a Rag2 deficient background. Cbl-b^{+/+}/Rag2^{Δ/Δ} deficient animals harbored a slightly increased fungal burden when compared to Cbl-b^{+/+}/Rag2^{+/+} mice, although this did not reach statistical significance. Importantly, it was again observed that deletion of Cbl-b in the absence of an adaptive immune system had a protective effect with regard to fungal colonization, as evidenced by the decreased fungal load in the skin of Cbl-b^{Δ/Δ}/Rag2^{Δ/Δ} mice, when compared to their Cbl-b^{+/+}/Rag2^{Δ/Δ} littermate controls (FIG. 3h). Thus, loss of Cbl-b in innate immune cells confers resistance to systemic as well as cutaneous Candida albicans infections.

### Example 4—Cbl-b Controls Anti-Fungal Activities of Dendritic Cells and Macrophages

Fungicidal activity within the innate immune system is mainly attributed to neutrophils, macrophages and DC (Drummond et al., 2015). To directly investigate the effect of Cbl-b deficiency on the anti-fungal activity of these cell types, in vitro killing assays were performed by co-culturing bone marrow neutrophils from uninfected mice, bone marrow-derived macrophages (BM-M), bone marrow-derived dendritic cells (BM-DC), bone marrow monocytes, and splenic DC, isolated from the bone marrow or the spleen, respectively, of uninfected mice, with Candida albicans. No significant difference in fungicidal activity could be observed, when comparing Cbl-b^{Δ/Δ}, Cbl-b^{C373A/C373A}, and Cbl-b^{+/+} bone marrow neutrophils (FIG. 5a). However, BM-M, and BM-DC, bone marrow monocytes and splenic DC, both isolated from uninfected mice, showed an increased potency to kill Candida albicans in the absence of Cbl-b or Cbl-b ligase function (FIG. 5b,c,d,e). Of note, rates of phagocytosis were comparable between BM-M or BM-DC of all three genotypes (FIG. 6a,b). Compatible with a more prominent role for Cbl-b in anti-fungal responses by DC and macrophages, higher levels of Cbl-b protein in BM-DC and BM-M were observed, when compared to bone marrow neutrophils, both in lysates from Candida albicans stimulated as well as in uninfected cells (FIG. 6c). Thus, Cbl-b controls the fungicidal activity of both BM-M and BM-DC as well as isolated bone marrow derived monocytes and splenic DC.

In line with previous observations, pharmacologic inhibition of phagocytosis using Dynasore abrogated BM-DC mediated killing. Likewise, antibody mediated blocking of Dectin-1 significantly reduced Candida albicans killing and reduced the increased fungal killing in both Cbl-b^{Δ/Δ} and Cbl-b^{C373A/C373A} BM-DC to levels observed in Cbl-b^{+/+} cells (FIG. 5f). In addition, pharmacologic inhibition of Syk with R406 almost completely abrogated Candida albicans killing (FIG. 5g). It was next measured whether a lack of Cbl-b or defective Cbl-b ligase function affects the ability of BM-M, BM-DC, bone marrow-derived monocytes, and splenic DC to produce ROS. Cbl-b^{Δ/Δ} or Cbl-b^{C373A/C373A} bone marrow neutrophils did not significantly differ from Cbl-b^{+/+} bone marrow neutrophils with respect to both the kinetics and amplitude of ROS production in response to Candida albicans (FIG. 6d). However, the increased fungicidal activity of Cbl-b^{Δ/Δ} or Cbl-b^{C373A/C373A} BM-M, BM-DC, bone marrow monocytes and splenic DC correlated with an increased Candida albi cans-induced ROS production as compared to Cbl-b sufficient cells (FIG. 6e-h). To test whether this de-regulated cellular response to fungal challenge is specific to stimulation with Candida albicans, BM-DC were stimulated with clinical isolates of Candida glabrata, Candida krusei, and Candida dubliniensis. It was again observed, that loss of Cbl-b significantly enhanced the production of ROS in response to stimulation with these distinct fungi (FIG. 6i-k). Thus, loss of Cbl-b enhances the immune response to several clinically relevant fungal pathogens.

Candida albicans mediated activation of BM-DC also triggers Syk dependent activation of the inflammasome, that enables cleavage of pro-interleukin-1β (IL-1β) by Caspase-β (Gringhuis et al., 2012; Zwolanek et al., 2014). Significantly increased activation of Caspase-(FIG. 7a) and enhanced release of mature IL-1β in Candida albicans stimulated Cbl-b^{Δ/Δ} or Cbl-b^{C373A/C373A} BM-DC were indeed observed when compared to control Cbl-b^{+/+} BM-DC, which could be reduced by a Dectin-1 blocking antibody (FIG. 7b). Pharmacologic inhibition of Syk also almost completely abrogated IL-1β secretion from BM-DC of all three genotypes (FIG. 7c). Similarly increased production and release of mature IL-1β from Candida albicans-stimulated Cbl-b′ ° or Cbl-b^{C373A/C373A} BM-M were found, when compared to Cbl-b^{+/+} BM-M (FIG. 7d).

To monitor the transcriptional response to Candida albicans infection, mRNA levels of pro-inflammatory cytokines were assessed. Significantly increased transcript levels of IL-1β, IL-6, TNF, IL-23, and IL-12 in Cbl-b^{Δ/Δ} and Cbl-b^{C373A/C373A} BM-DC when compared to Cbl-b^{+/+} control cells were observed (FIG. 7e). This increase in cytokine mRNA expression correlated with significantly increased secretion of the pro-inflammatory cytokines IL-6 and TNF by Cbl-b^{Δ/Δ} and Cbl-b^{C373A/C373A} BM-DC when compared to BM-DC (FIG. 7f,g). Blocking Dectin-1 reduced the release of IL-6 and TNF for all three genotypes and, importantly, also reduced the increased cytokine release in the Cbl-b deficient BM-DC (FIG. 7f,g). Again, pharmacologic inhibition of Syk almost completely abrogated the release of IL-6 and TNF by Candida albicans stimulated BM-DC of all three genotypes (FIG. 7h,i). Candida albicans stimulation also led to a substantially increased release of IL-23 and IL-12 by BM-DC (FIG. 7j,k) and IL-6 and TNF by BM-M (FIG. 7l,m), when Cbl-b^{Δ/Δ} and Cbl-b^{C373A/C373A} cells were compared to Cbl-b^{+/+} control cells. Additionally the secretion of the anti-inflammatory mediator IL-1 receptor antagonist (IL-1Ra) by Candida albicans stimulated BM-DC was tested. A significant increase in secretion of IL-1Ra was observed when Cbl-b^{Δ/Δ} and Cbl-b^{+/+} BM-DC were compared (FIG. 7n).

To further test for the contribution of Dectin-1, cytokine secretion by BM-DC of all three genotypes in response to increasing doses of zymosan, a Candida albicans cell wall homogenate, or curdlan, a purified β-glucan was compared. Stimulation with zymosan and the Dectin-1 agonist curdlan resulted in an increased release of IL-1β, IL-6, and TNF by Cbl-b^{Δ/Δ} and Cbl-b^{C373A/C373A} BM-DC (FIG. 8a-f). Since loss of Cbl-b did not affect the mortality rates in lethal Listeria monocytogenes infections (see FIG. 2d), a potential role of Cbl-b in the response of BM-DC to stimulation with the TLR4 agonist Lipopolysaccharide (LPS) or the TLR9 agonist CpG was tested, both important pathogen associated molecular pattern receptors involved in Listeria monocytogenes infections. Importantly, no significant differences in the secretion of the pro-inflammatory cytokines IL-6 and TNF in response to stimulation with a high or low dose of LPS or CpG were observed, when Cbl-b^{Δ/Δ} and Cbl-b^{+/+} BM-DC were compared (FIG. 8g-j). Thus, Cbl-b is a key regulator of Dectin-1/Syk-mediated responses of BM-DC and BM-M to Candida albicans infections.

The data so far showed that Cbl-b deficiency or a lack of Cbl-b activity leads to a severe de-regulation of the anti-fungal immune response in vitro. To address, whether the observed differences could be recapitulated in immune cells recruited to inflammatory sites, Cbl-b^{Δ/Δ} and Cbl-b^{+/+} mice were challenged by intraperitoneal infection with Candida albicans and their anti-fungal activity was assessed 24 h later. Loss of Cbl-b led to an enhanced ROS response by total infiltrating immune cells into the peritoneal cavity. Increased ROS production was already observed in Cbl-b^{Δ/Δ}, when compared to Cbl-b^{+/+}, infiltrating cells without further re-stimulation with Candida albicans (FIG. 5h,i). Likewise, the fungicidal activity of Cbl-b^{Δ/Δ} immune infiltrates was increased, when compared to immune cell infiltrates from Cbl-b^{+/+} littermate control mice (FIG. 5j). Intraperitoneal infection with Candida albicans leads to the recruitment of both inflammatory monocytes/macrophages and neutrophils. To address the contribution of either cell type to the observed differences, neutrophils and monocytes/macrophages were FACS purified from total infiltrating immune cells and tested for their ability to produce ROS after re-stimulation with Candida albicans. An increase in ROS production by Cbl-b^{Δ/Δ} monocytes/macrophages was observed, but not neutrophils, recruited to the peritoneal cavity when compared to Cbl-b^{+/+} cells (FIG. 5k,l). Additionally, intraperitoneal lavages were tested for the inflammatory cytokines IL-6 and TNF, as well as IL-1Ra. As seen in the in vitro experiments, an increase in both IL-6 and TNF was observed, as well as IL-1Ra, when peritoneal lavages from Cbl-b^{Δ/Δ} and Cbl-b^{+/+} mice were compared (FIG. 8k).

The data indicated that the protective effect of Cbl-b deficiency to fungal challenge was mediated by tissue resident or recruited phagocytic immune cells, but not neutrophils. To test for neutrophil intrinsic effects phagocytic immune cells were depleted in vivo using clodronate liposomes. Clodronate liposomes have been reported to deplete macrophages, sub-populations of monocytes, and subsets of DC, while leaving neutrophils unaffected (Buiting et al., 1994; Buiting et al., 1996; Qian et al., 1994; Sunderkotter et al., 2004; van Rooijen et al., 1992). Phagocytes were depleted 24 h before, and 24 h after systemic infection with Candida albicans and both weight loss and survival were monitored over time. Depletion of phagocytes led to a significant increase in weight loss in both Cbl-b^{+/+} and Cbl-b^{Δ/Δ} mice compared to the control PBS liposome treated cohorts (Qian et al., 1994) (FIG. 5m). Importantly, there was no significant difference between Cbl-b^{Δ/Δ} and Cbl-b^{+/+} animals treated with clodronate liposomes (FIG. 5m). Likewise, while Cbl-b deficiency led to a prolonged survival when PBS liposome treated Cbl-b^{Δ/Δ} and Cbl-b^{+/+} cohorts were compared, depletion of phagocytes by clodronate liposomes completely abolished any survival benefit due to the loss of Cbl-b (FIG. 5n). These data show that Cbl-b deficiency in phagocytic cells is critical for the observed beneficial effect to systemic Candida albicans infections.

### Example 5—Loss of Cbl-b Enhances CLR-Mediated Signaling in Response to Candida albicans

To assess whether loss of Cbl-b or Cbl-b ligase function altered the magnitude of the signaling response mounted by BM-DC, tyrosine phosphorylation was analyzed 30, 60, and 90 minutes after stimulation with Candida albicans. A strong increase in tyrosine phosphorylation in response to Candida albicans stimulation was observed in lysates prepared from Cbl-b^{Δ/Δ} and Cbl-b^{C373A/C373A} BM-DC, when compared to control Cbl-b^{+/+} BM-DC for all stimulation time points tested (FIG. 9a). Canonical signaling by CLRs involves proximal activation of Src kinase and Shp-2 that subsequently results in the activation of Syk (Deng et al., 2015). Comparable Src tyrosine phosphorylation was observed after stimulation of BM-DC with Candida albicans, but increased levels of phosphorylated Shp-2 and Syk (FIG. 9b). Due to the increased anti-fungal effects caused by Syk inhibition as compared to Dectin-1 blockade, the role of Cbl-b deletion in Dectin-1, Dectin-2/3, and Mincle signaling was further tested. Although these surface receptors were expressed at comparable levels in Cbl-b^{Δ/Δ} and Cbl-b^{+/+} BM-DC (FIG. 10a), an increase in Syk and Shp-2 phosphorylation in response to heat killed Candida albicans (HK; to activate Dectin-1), Candida albicans hyphae (Hyph; to activate Dectin-2/3), as well as Trehalose-6,6-dimycolate (TDM; to activate Mincle) in the absence of Cbl-b (FIG. 9c) was observed. Additionally, an increase in phosphorylation of the distal CLR signaling molecules PLCy2 and p65 NF-κB in Candida albicans-stimulated Cbl-b^{Δ/Δ} and Cbl-b^{C373A/C373A} BM-DC was observed (FIG. 9d). In agreement with the immunoblots, Cbl-b^{Δ/Δ} BM-DC showed increased levels of both phosphorylated Shp-2 (FIG. 9e) and phosphorylated Syk (FIG. 9f) following stimulation with Candida albicans, as analyzed by confocal microscopy. These data identify a central function for Cbl-b in regulating proximal CLR signaling in response to Candida albicans infection.

### Example 6—Cbl-b Targets Syk and Dectin-1, and -2 for Ubiquitination

To test whether the E3 ligase Cbl-b could poly-ubiquitinate molecules critical for proximal CLR signaling, ubiquitination assays with recombinant proteins were performed. No poly-ubiquitination of recombinant Shp-2 was detected in these assays, but observed robust poly-ubiquitination of recombinant active Syk was observed (FIG. 11a). The chain linkage type formed by Cbl-b was analyzed by mass spectrometry and unique peptides (FAGKQLED) (SEQ ID NO: 36) derived specifically from K48-linked ubiquitin chains were identified (FIG. 10b). Cbl-b substrate specificity is mainly mediated by the amino-terminal tyrosine kinase binding (TKB) domain. In agreement with this notion, a truncated Cbl-b protein (Cbl-b²⁹⁻⁴⁸³) lacking the carboxy-terminal ubiquitin associated (UBA) domain, but containing the TKB and RING finger domains, still exhibited poly-ubiquitination activity towards Syk (FIG. 11a). As expected, the C373A mutant of Cbl-b²⁹⁻⁴⁸³ did not show Syk poly-ubiquitination activity (FIG. 11a). Importantly, non-phosphorylated recombinant Syk showed strongly diminished poly-ubiquitination by Cbl-d²⁹⁻⁴⁸³ (FIG. 11b), consistent with Cbl-b targeting activated Syk. To test for interactions between Syk and Cbl-b, co-immunoprecipitation experiments were performed. Increased co-immunoprecipitation of Syk with Cbl-b after stimulation of BM-DC with Candida albicans could be observed (FIG. 11c). Upon treatment with the proteasome blocker MG132, an increase in phosphorylated Syk, but not total Syk, was observed in lysates from Cbl-b^{+/+} BM-DC stimulated with Candida albicans. Importantly, levels of phosphorylated Syk did not change upon MG132 treatment of Cbl-b^{Δ/Δ} BM-DC stimulated with Candida albicans (FIG. 11d), indicating that Cbl-b mediates its effects via proteasomal targeting. These results show that Cbl-b can ubiquitinate active Syk.

To test for potential additional effects of Cbl-b deletion on proximal signaling initiated by CLR stimulation, the question was raised whether the CLR family members Dectin-1, and -2 might also serve as targets for Cbl-b mediated degradation. Thus, BM-M were stimulated with Candida albicans or Candida albicans hyphae, and tested for Dectin-1, and -2 protein levels, respectively, at various time points after stimulation. While Dectin-1, and -2 levels decreased over time in control cells, their expression levels remained constant in the absence of Cbl-b (FIG. 10c,d). To test whether Dectin-1, and -2 are ubiquitinated in response to fungal stimulation, and if so, whether ubiquitination is dependent on Cbl-b, Cbl-b^{Δ/Δ} and Cbl-b^{+/+} cells were stimulated, Dectin-1, or -2 were immuno-precipitated and ubiquitin was blotted. Ubiquitination of Dectin-1 and Dectin-2 in response to stimulation with Candida albicans or Candida albicans hyphae, respectively, was observed. This ubiquitination was reduced in the absence of Cbl-b (FIG. 10e,f). These data show that Cbl-b regulates signaling of CLRs by both regulating the protein levels of Dectin-1, and -2, as well as modulating the proximal signaling molecule Syk.

### Example 7—Inhibition of Cbl-b Enhances Anti-Fungal Immunity In Vitro and In Vivo

Based on the genetic and biochemical data it was reasoned that blocking Cbl-family TKB-mediated substrate recognition might de-regulate the activation of Syk downstream of CLR stimulation. To test this hypothesis, a fusion peptide consisting of a cell penetrating peptide derived from the Drosophila Antennapedia homeodomain was designed (May et al., 2000) and a 16aa long phosphorylated peptide derived from Syk that encompasses Tyrosine 317 (Y317), reported to be required for Cbl-family-ligase mediated targeting of Syk (Sohn et al., 2003; Yankee et al., 1999; Zou et al., 2009), and the surrounding flanking regions (FIG. 11e). Attachment of the Antennapedia peptide has been reported to allow for the efficient delivery of attached peptides into the cytosol (Jones et al., 2005). Pre-incubation of recombinant Cbl-b with this Cbl-family TKB-binding peptide completely abrogated in vitro ubiquitination of active Syk (FIG. 11f). Since activation of ROS production in BM-DC in response to fungal stimulation is Syk dependent, ROS production in the presence of the novel Cbl-b-TKB binding peptide was monitored. Intriguingly, peptide interference massively increased ROS production by BM-DC in response to Candida albicans stimulation (FIG. 11g). The observed effect even surpassed the increase in ROS production in Cbl-b^{Δ/Δ} cells. Since the peptide interference strategy potentially inhibits TKB mediated targeting of substrate proteins by all Cbl family ligases (c-Cbl, Cbl-b, and Cbl-3), it appears that Cbl family ligases could act in an at least partly redundant manner. In agreement with such a scenario, a significantly increased sensitivity to peptide interference in BM-DC lacking Cbl-b when compared to Cbl-b sufficient cells was observed (FIG. 10g). Most importantly, the Cbl-family TKB binding peptide also increased the fungicidal activity of BM-DC upon Candida albicans infection in vitro (FIG. 11h). Of note, addition of the Antennapedia peptide alone, or a phosphorylated scrambled peptide, did not affect ROS production, or the fungicidal activity of BM-DC.

Due to the observed in vitro efficacy of the inhibitory peptide it was investigated whether inhibition of Cbl family ligases might represent a novel strategy to increase immune reactivity and function towards fungal sepsis in vivo. Thus, Cbl-b^{Δ/Δ} and littermate control mice were infected with a lethal dose of Candida albicans and treated two times with a high dose of the inhibitory Cbl-family TKB-binding peptide (100 μg, i.p., day 0 and day 2), or left untreated. Strikingly, treating mice only twice had a significant protective effect on wild type Cbl-b^{+/+} mice, as evidenced by reduced weight loss and delayed mortality, when compared to the untreated control cohort (FIG. 12a,b). Additionally, peptide treatment led to a decrease in blood urea nitrogen levels, indicative of reduced kidney damage (FIG. 12c). Peptide treatment did not confer any protective effect on Cbl-b^{Δ/Δ} mice (FIG. 10h). Reducing the concentration of the TKB binding inhibitor by 5-fold (20 μg per injection) yielded comparable results (FIG. 12d,e; FIG. 10i). In line with the decreased morbidity observed upon low dose peptide treatment, a significantly reduced fungal load in the kidneys of peptide treated mice was detected (FIG. 12f). Treating mice with the Antennapedia peptide or a scrambled phosphorylated control peptide did not affect morbidity or mortality, when compared to the untreated control cohort. Due to the protective function of peptide treatment in Cbl-b^{+/+} mice, the therapeutic potential of the TKB-binding peptide in already established fungal sepsis was finally tested. Thus, mice were treated on day 2 and day 3 (100 μg per i.p. injection) after infection with a lethal dose of Candida albicans. Strikingly, this treatment regimen completely blocked further weight loss, significantly reduced kidney fungal load 2 days after the second peptide treatment, and even protected mice from mortality (FIG. 12g,h,i). Histopathologic analyses confirmed these findings, showing significantly reduced renal inflammation (FIG. 12j,k), fungal burden (FIG. 10j), and neutrophil infiltration (FIG. 10k). Thus, the Cbl-family TBK binding peptide enhances anti-fungal immunity and can protect from lethal Candida albicans sepsis.

### Example 8—CBL-Family TKB-Domain Binding Peptide Inhibits Both CBL-B and CBL in Murine DC

Active SYK is targeted for proteasomal degradation by both CBL-B and CBL in activated B cells (Kitaura, 2007; Sohn, 2003). The CLB-B TKB-domain binding peptide is derived from SYK and could thus serve as a peptide binding and inhibiting both CLB-B and CBL. As observed in FIG. 5g, stimulation of cblb^{+/+} BM-DC with Candida albicans in the presence of the CBL-B TKB-binding peptide (M1) led to a marked increase in ROS production when compared to controls that were not treated with the peptide. An increase in ROS production was still detectable in cblb^{Δ/Δ} BM-DC in cells that were treated with peptide (M1) when compared to untreated control cells. To test, whether the additional effect of peptide treatment on ROS production in cblb^{Δ/Δ} cells was mediated by inhibition of CBL, we derived Hoxb8-hematopoietic progenitors (Redecke, 2013) from cblb^{+/+} and cblb^{Δ/Δ} animals and introduced an shRNAs targeting either cbl or a control shRNA targeting renilla luciferase as a negative control. Hoxb8-hematopoietic progenitors sufficient for CBL-B, or lacking either CBL-B or both CBL and CBL-B were differentiated into dendritic cells (DC) and assayed for ROS production upon Candida albicans infection in the presence or absence of the CBL-B TKB-domain binding peptide (M1), subsequently. ROS production upon Candida albicans infection was increased in cblb^{Δ/Δ} DC, when compared to cblb^{+/+} cells. Deletion of both CBL-B and CBL further increased the production of ROS by DC when compared to CBL-B single deficient cells. Importantly, while CBL-family TKB-domain binding peptide (M1) treatment increased ROS production in cblb^{+/+} and cblb^{Δ/Δ} DC, it did not further increase ROS production in cells lacking both CBL-B and CBL (FIG. 13a). These results argue for an at least partially redundant function for CBL-B and CBL in regulating the SYK mediated production of ROS in response to Candida albicans infection. Furthermore, the CBL-family TKB-domain binding (M1) peptide appears to act on both CBL-B and CBL, since peptide treatment of Candida albicans infected cells deficient in both CBL-B and CBL did not result in a further upregulation of ROS production, when compared to peptide untreated controls. Peptide treatment thus phenocopies CBL/CBL-B double deficiency in SYK mediated ROS production in DC.

### Example 9—CBL-Family TKB-Domain Binding Peptide Treatment Enhances the Proliferative Response of Murine CD4⁺ T Cells

To test for the applicability of the CBL-family TKB-binding peptide to modulate cellular responses other than anti-fungal immune reactions as observed in myeloid immune cells, we carried out T cell proliferation assays, since T cell proliferation has been reported to be regulated by CBL-B in murine T cells, previously (Bachmaier, 2000; Chiang, 2000). We observed a dose dependent increase in the proliferative response of murine cblb^{+/+} CD4⁺ T cells in the presence of the CBL-family TKB-binding peptide (M3; FIG. 14a) in response to stimulation with either plate bound CD3 or plate bound CD3 plus soluble CD28 antibodies when compared to cblb^{+/+} CD4⁺ cells without peptide treatment. A scrambled variant of the CBL-family TKB domain-binding peptide (M4), with an identical amino acid composition and a central phospho-tyrosine fused to the unaltered antennapedia peptide, was used as a control and only mildly increased cblb^{+/+} CD4⁺ T cell proliferation when compared to peptide untreated cells (FIG. 14b). Peptide (M3) mediated inhibition of CBL-B activity showed more pronounced effects under suboptimal stimulatory conditions, i.e. low concentrations of CD3, both in the presence or absence of CD28 mediated co-stimulatory signals (FIG. 14a, b).

Analogous to the partly redundant regulation of B cell receptor triggered responses by Cbl family members (Kitaura, 2007), deficiency of both CBL-B and CBL in murine T cells further enhances T cell proliferation in response to T cell receptor stimulation (Naramura, 2002). In agreement with these data and a dual function for the CBL-family TKB-domain binding peptide, inhibiting both CBL-B and CBL, stimulation of CD4⁺ T cells, derived from cblb^{Δ/Δ} mice, led to a marked increase in the proliferative response in the presence of the TKB domain-binding peptide (M3), when compared to untreated control cells (FIG. 14c). Treatment of cells with scrambled control peptides again only mildly increased the proliferative response of cblb^{Δ/Δ} T cells when compared to peptide untreated control cells (FIG. 14d). In summary, the SYK derived CBL-family TKB domain-binding peptide functions as both an inhibitor of CBL-B and CBL in C-type lectin receptor mediated ROS production by DC, as well as T cell receptor mediated cellular proliferation in murine CD4⁺ T cells.

### Example 10—CBL-Family TKB-Domain Binding Peptide Treatment Enhances the In Vivo Anti-Tumor Immune Response in a Melanoma Model

Cblb is a checkpoint regulator of anti-tumor immune responses and cblb^{Δ/Δ} T cells were shown to mount an enhanced immune response towards tumors in various animal tumor models (Loeser, J E M, 2007; Chiang, 2007). To test for the efficacy of CBL-family TKB-domain binding peptide mediated immune modulation in a cancer immune response, we inoculated C57Bl/6 mice with 10⁶ B16-SIY melanoma cells subcutaneously and treated mice with engrafted tumors every other day with vehicle, 100 μg scrambled peptide (M2), or 100 μg CBL-family TKB-domain binding peptide (M1) and recorded tumor volume and bodyweight over time. While peptide treatment did not affect bodyweight gain over the observed period of time, we observed a significantly reduced tumor volume in CBL-family TKB-domain binding peptide (M1) treated mice, but not scrambled peptide (M2) treated mice, when compared to the vehicle treated control cohort (FIG. 15a, b). These results provide evidence for the efficacy of the TKB domain binding peptide treatment not only in anti-fungal immune responses, but also in the modulation of anti-tumor immunity.

### Example 11—CBL-Family TKB-Domain Binding Peptide Treatment Enhances the Proliferation and Activation of Human T Cells

CBL-B silencing experiments in human cells indicated similar immune-modulatory functions for CBL-B in mouse and human T cells (Pedraza-Alva, 2011; Stromnes, 2010) and CBL-B holds promise as a target for checkpoint blockade inhibition in human cancer. To test for the efficacy of peptide mediated inhibition of CBL-B in human T cells, we engineered a humanized variant of the CBL-B TKB-domain binding peptide used in murine assays (peptide H1). In agreement with results obtained with murine T cells, we observed a peptide (H1) dose dependent significant increase in the proliferative response of human CD4⁺ and CD8⁺ T cells in response to stimulation with plate bound CD3/CD28 antibodies (FIG. 16a, b). Likewise, production of the proinflammatory cytokine IFN-γ by CD4⁺ T cells increased significantly in the presence of the human CBL-B TKB-binding peptide (H1; FIG. 16c). In agreement with peptide treatment modulating T cell receptor stimulation, we additionally observed a dose dependent upregulation of the activation marker CD69 on T cells upon treatment with the humanized CBL-family TKB domain binding peptide (H1), when compared to untreated control cells (FIG. 16d). Slight effects of scrambled control peptide (H2) were noted, but were substantially smaller than effects observed in humanized CBL-family TKB domain binding peptide treated cells.

### REFERENCES

Armstrong-James et al. Trends in microbiology 22, 120-127 (2014).
Bachmaier et al. Nature 403, 211-216 (2000).
Bechara et al. FEBS letters 587, 1693-1702 (2013).
Bourgeois et al. Methods in molecular biology 470, 125-139 (2009).
Boxio et al. Journal of leukocyte biology 75, 604-611 (2004).
Brown et al. Nature 413, 36-37 (2001).
Brown et al. Science translational medicine 4.165 (2012):165rv113.
Brown et al. Trends in microbiology 22, 107-109 (2014).
Brugidou et al. Biochemical and Biophysical Research Communications 214(2):685 (1995).
Buiting et al. Journal of drug targeting 2, 357-362 (1994).
Buiting et al. Journal of immunological methods 192, 55-62 (1996).
Chiang et al. Nature 403, 216-220 (2000).
Chiang et al. The Journal of Clinical Investigation 117(4):1029 2007
Chiba et al. eLIFE 1-20 (2014).
Deng et al. Nature immunology 16, 642-652 (2015).
Denning Clin Infect Dis 26(4), 781-803 (1998).
Dorfer et al. Journal of proteome research 13, 3679-3684 (2014).
Drummond et al. Cold Spring Harbor perspectives in medicine 5(2015).
Enoch et al. Journal of Medical Microbiology 55, 809-818 (2006).
Fellmann et al., Cell Reports 5, 1704-1713 (2013).
Gringhuis et al. Nature immunology 13, 246-254 (2012).
Gross et al. Nature 459, 433-436 (2009).
Gustin et al. Journal of immunology 177, 5980-5989 (2006).
Han et al. Nature immunology 11, 734-742 (2010).
Igyarto et al. Immunity 35, 260-272 (2011).
Jain et al. J Pharm Bioallied Sci. 2(4), 314-320 (2010).
Jones et al. British journal of pharmacology 145, 1093-1102 (2005).
Kashem et al. Immunity 42, 356-366 (2015).
Kawai et al. Nature Immunology 11, 373-384 (2010).
Kawai et al. Immunity 34(5), 637-650 (2011).
Kawai et al., Biochimica et Biophysica Acta. Molecular Cell Research, 1853(5), 2015: 873-880
Kitaura et al. Immunity 26, 567-578 (2007).
Kullberg et al. The New England journal of medicine 373, 1445-1456 (2015).
Kumar et al., Journal of Medicinal Chemistry, 55(7) 2012: 3583-3587
Lanternier et al. Current opinion in pediatrics 25, 736-747 (2013).
LeibundGut-Landmann et al. Nature immunology 8, 630-638 (2007).
Lim et al. Nature Communications 6, 1-13 (2015).
Lindsay Curr. Opin. Pharmacol. 2, 587-594 (2002).
Loeser et al. Cell cycle 6, 2478-2485 (2007).
Loeser et al. JEM 204(4), 879-891 (2007).
Lortholary et al. Clinical microbiology reviews 10, 477-504 (1997).
Mancini et al. Journal of Biological Chemistry 277, 14635-14640 (2002).
May et al. Science 289, 1550-1554 (2000).
Mocsai et al. Nature reviews. Immunology 10, 387-402 (2010).
Nakao et al. Molecular and cellular biology 29, 4798-4811 (2009).
Naramura et al. Nature Immunology 3(12), 2002, 1192.
Ohno et al. Archives of Biochemistry and Biophysics 594, 1-7 (2016).
Paolino et al. Journal of immunology 186, 2138-2147 (2011).
Paolino et al. Nature 507, 508-512 (2014).
Park et al. Aids 23, 525-530 (2009).
Pedraza-Alva et al. Life, 63(10): 940-948 (2011).
Peschard et al. Mol. Cell 8, 995-1004 (2001).
Peschard et al. Journal of Biological Chemistry 279, 29565-71 (2004).
Qian et al. Journal of immunology 152, 5000-5008 (1994).
Qu et al. Blood 103, 1779-1786 (2004).
Ravikumar et al. Frontiers in microbiology 6, 1322 (2015).
Redecke et al., nature methods 10(8) 2013, 795
Ruland et al. Annals of the New York Academy of Sciences 1143, 35-44 (2008).
Ruzza et al., Expert Opinion on Therapeutic Patents, Informa Healthcare, GB, 19(10) 2009: 1361-1376
Snyder et al. PLoS Biology 2(2) 0186 (2004).
Sohn et al. The Journal of experimental medicine 197, 1511-1524 (2003).
Spranger et al., Journal for ImmunoTherapy of Cancer 2014, 2:3
Stromnes et al., The Journal of Clinical Investigation 2010 120(10), 3722.
Sunderkotter et al. Journal of immunology 172, 4410-4417 (2004).
Taus et al. Journal of proteome research 10, 5354-5362 (2011).
Tugyi et al. PNAS 102(2) 413-418 (2005).
Tsygankov et al. Oncogene 20, 6382-6402 (2001).
van Rooijen et al. Research in immunology 143, 215-219 (1992).
Wallner et al. PloS one 8, e65178 (2013).
Wang et al., Cancer Cell 31, 532-548 (2017).
Wirnsberger et al. Nature genetics 46, 1028-1033 (2014).
Yankee et al. Journal of immunology 163, 5827-5835 (1999).
Zou et al. The Journal of biological chemistry 284, 18833-18839 (2009).
Zwolanek et al. PLoS pathogens 10, e1004525 (2014).

All documents cited herein are included herein by reference in their entirety.

1. A fusion peptide comprising: a cell delivery portion and a casitas b-lineage lymphoma-family (Cbl-family) tyrosine kinase binding (TKB) domain binding peptide.

2. The fusion peptide of claim 1, wherein the cell delivery portion is a cell penetrating peptide.

3. The fusion peptide of claim 1, wherein the Cbl-family TKB domain binding peptide is a peptide fragment of spleen tyrosine kinase (Syk).

4. The fusion peptide of claim 1, wherein the Cbl-family TKB domain binding peptide is a Cbl-b binding peptide, preferably a binding peptide of the TKB domain of Cbl-b.

5. The fusion peptide according to claim 1, wherein the Cbl-family TKB domain binding peptide has the sequence motif N/DXpY, preferably N/DXipYX2P, wherein X, X1 and X2 are selected from any amino acid, N/D is either asparagine (N) or aspartic acid (D), and pY is phosphotyrosine, wherein the sequence motif optionally contains one or more of its amino acids in D-form or in retro-inverso-form.

6. The fusion peptide according to claim 1, wherein the Cbl-family TKB domain binding peptide comprises the following consensus sequence:
SFNPYEPX₁X₂X₃,
wherein X₁-X₃ are selected independently from any amino acid, preferably wherein the Cbl-family TKB domain binding peptide is a peptide fragment of Syk, more preferably wherein the Cbl-family TKB domain binding peptide comprises the sequence SFNPYEPTGG, SFNPYEPTGG, SFNPYEPEVA or SFNPYEPELA, wherein any of these sequences optionally contains one or more of its amino acids in D-form or in retro-inverso-form.

7. The fusion peptide according to claim 6, wherein the tyrosine in said sequence is phosphorylated.

8. The peptide according to claim 1, wherein the Cbl-family TKB domain binding peptide is of Syk and wherein the peptide is a regulator of innate immunity, preferably a regulator of innate anti-parasitic, anti-fungal or anti-tumor immunity.

9. A pharmaceutical composition, comprising the fusion peptide or the Cbl-family TKB domain binding peptide as set forth in claim 1, and a pharmaceutically acceptable carrier promoting cellular uptake of said peptide, preferably wherein the pharmaceutically acceptable carrier is selected from vesicles, micelles, liposomes and microsomes, more preferably wherein the pharmaceutical composition is prepared for parenteral administration, especially intravenous, intraarterial, intramuscular, intrathecal, subcutaneous or intraperitoneal administration or for topical administration, preferably further comprising a skin penetration enhancer.

10. The peptide or pharmaceutical composition according to claim 1, for use in therapy, wherein the peptide or composition is administered to a patient, preferably wherein the peptide is a Cbl-family TKB domain binding peptide of Syk.

11. The peptide or pharmaceutical composition for use according to claim 10 for use in the prevention or treatment of tumors, cancer, viral infections, bacterial infections, fungal infections, parasitic infections, especially intracellular parasites, or drug-induced immunosuppression in a patient.

12. A Cbl-family protein inhibitor for use in therapy in a patient as a vaccine adjuvant, preferably wherein the Cbl-family protein inhibitor is a Cbl-family TKB domain binding peptide, more preferably a Cbl-family TKB domain binding peptide of Syk.

13. A Cbl-family protein inhibitor for use in the prevention or treatment of a fungal infection in a patient, wherein the fungal infection is a yeast infection, preferably a candidiasis or a cryptococcosis.

14. A Cbl-family protein inhibitor for use in the prevention or treatment of a fungal infection in a patient, wherein the Cbl-family inhibitor comprising a Cbl-family binding peptide of Syk.

15. The Cbl-family protein inhibitor or peptide or pharmaceutical composition for use according to claim 10, wherein the fungal infection is a yeast infection, preferably a candidiasis, especially a Candida albicans, Candida glabrata, Candida krusei or a Candida dubliniensis infection, a dermatophyte or an aspergillus infection.

16. A vaccine, comprising as an adjuvant a Cbl-family protein inhibitor, preferably wherein the Cbl-family protein inhibitor comprises a Cbl-family TKB domain binding peptide of Syk, and at least one antigen, preferably the Cbl-family protein inhibitor being defined as claim 1.

17. A kit comprising the peptide according to claim 1 and a pharmaceutically acceptable excipient, preferably suitable for intracellular administration in the patient.

18. An in vitro or in vivo method for stimulating or activating an immune cell, comprising contacting or treating the cell with a fusion peptide or a Cbl-family TKB domain binding peptide as set forth in claim 1, preferably wherein cytokines are produced, more preferably wherein the cytokines are selected from IL-6, IL-1β, IL-23 and TNF.


SEQUENCE LISTING (46 sequences)

SEQ ID NO: 1 - protein, 10 aa, Artificial Sequence
  consensus sequence
  VARIANT 8..10: X is any amino acid
SFNPYEPXXX

SEQ ID NO: 2 - protein, 10 aa, Mus musculus
  also in Rattus norvegicus
SFNPYEPTGG

SEQ ID NO: 3 - protein, 10 aa, Macaca mulatta
SFNPYEPEVA

SEQ ID NO: 4 - protein, 10 aa, Homo sapiens
SFNPYEPELA

SEQ ID NO: 5 - protein, 16 aa, Artificial Sequence
  consensus sequence
  VARIANT 11,12,13,14,16: X is any amino acid
STVSFNPYEPXXXXWX

SEQ ID NO: 6 - protein, 16 aa, Mus musculus
STVSFNPYEPTGGPWG

SEQ ID NO: 7 - protein, 16 aa, Rattus norvegicus
STVSFNPYEPTGGAWG

SEQ ID NO: 8 - protein, 16 aa, Macaca mulatta
STVSFNPYEPEVAPWA

SEQ ID NO: 9 - protein, 16 aa, Homo sapiens
STVSFNPYEPELAPWA

SEQ ID NO: 10 - protein, 17 aa, Drosophila melanogaster
DRQIKIWFQNRRMKWKK

SEQ ID NO: 11 - protein, 13 aa, Human immunodeficiency virus 1
GRKKRRQRRRPPQ

SEQ ID NO: 12 - protein, 27 aa, Mammalia
GWTLNSAGYLLGKINLKALAALAKKIL

SEQ ID NO: 13 - protein, 11 aa, Artificial Sequence
  REPEAT 1-11: 1 to 5 times repeat
  consensus sequence
  VARIANT 10: X is K or R
KKDKKDERRXK

SEQ ID NO: 14 - protein, 12 aa, Artificial Sequence
  REPEAT 1..11: 1 to 5 times repeat
  consensus sequence
  VARIANT 5: X is V, I or T
KIKKXBKKKGRK

SEQ ID NO: 15 - protein, 5 aa, Artificial Sequence
  sequence motif
  VARIANT 1: X is N or D
  VARIANT 2,4: X is any amino acid
  MOD_RES 3: PHOSPHORYLATION
XXYXP

SEQ ID NO: 16 - protein, 5 aa, Mus musculus
  and in Homo sapiens
DGYTP

SEQ ID NO: 17 - protein, 5 aa, Mus musculus
  and in Homo sapiens
DGYMP

SEQ ID NO: 18 - DNA, 20 bp, Artificial Sequence
  targeting Clec4n
ctgcccaaatcactggaagt

SEQ ID NO: 19 - DNA, 20 bp, Artificial Sequence
  targeting Clec4n
gaaattctgctccgcttcag

SEQ ID NO: 20 - DNA, 20 bp, Artificial Sequence
  targeting Clec7a
gaaccacaagcccacagaat

SEQ ID NO: 21 - DNA, 20 bp, Artificial Sequence
  targeting Clec7a
taggaaggcaaggctgagaa

SEQ ID NO: 22 - DNA, 20 bp, Artificial Sequence
  targeting Clec4d
tacgctggacgagaggaagt

SEQ ID NO: 23 - DNA, 20 bp, Artificial Sequence
  targeting Clec4d
acaggacagcaggtccaagt

SEQ ID NO: 24 - DNA, 20 bp, Artificial Sequence
  targeting Clec4e
caagtgctctcctggacgat

SEQ ID NO: 25 - DNA, 20 bp, Artificial Sequence
  targeting Clec4e
ctgtaagttctgcccggaaa

SEQ ID NO: 26 - DNA, 22 bp, Artificial Sequence
  targeting TNF
caaaattcgagtgacaagcctg

SEQ ID NO: 27 - DNA, 18 bp, Artificial Sequence
  targeting TNF
gagatccatgccgttggc

SEQ ID NO: 28 - DNA, 23 bp, Artificial Sequence
  targeting IL-6
gaggataccactcccaacagacc

SEQ ID NO: 29 - DNA, 24 bp, Artificial Sequence
  targeting IL-6
aagtgcatcatcgttgttcataca

SEQ ID NO: 30 - DNA, 20 bp, Artificial Sequence
  targeting IL-12
ggaagcacggcagcagaata

SEQ ID NO: 31 - DNA, 24 bp, Artificial Sequence
  targeting IL-12
aacttgagggagaagtaggaatgg

SEQ ID NO: 32 - DNA, 20 bp, Artificial Sequence
  targeting IL-23
gcagattccaagcctcagtc

SEQ ID NO: 33 - DNA, 20 bp, Artificial Sequence
  targeting IL-23
ttcaacatatgcaggtccca

SEQ ID NO: 34 - DNA, 25 bp, Artificial Sequence
  targeting IL-1beta
caaccaacaagtgatattctccatg

SEQ ID NO: 35 - DNA, 21 bp, Artificial Sequence
  targeting IL-1beta
gatccacactctccagctgca

SEQ ID NO: 36 - protein, 8 aa, Artificial Sequence
  peptide
FAGKQLED

SEQ ID NO: 37 - DNA, 97 bp, Artificial Sequence
  97mer oligonucleotide for generation of shRNAs targeting cbl
tgctgttgacagtgagcgctacgacgatgatgatgatgaatagtgaagccacagatgtattcatcatcatcatcgtcgtaatgcctactgcctcgga

SEQ ID NO: 38 - DNA, 97 bp, Artificial Sequence
  97mer oligonucleotide for generation of shRNAs targeting renilla luciferase
tgctgttgacagtgagcgcaggaattataatgcttatctatagtgaagccacagatgtatagataagcattataattcctatgcctactgcctcgga

SEQ ID NO: 39 - DNA, 24 bp, Artificial Sequence
  mir-E fwd
agaaggctcgagaaggtatattgc

SEQ ID NO: 40 - DNA, 30 bp, Artificial Sequence
  mir-E rev
gctcgaattctagccccttgaagtccgagg

SEQ ID NO: 41 - protein, 33 aa, Mus musculus
  antennapedia CPP - CBL-B TKB domain binding peptide
  MOD_RES 25: PHOSPHORYLATION
DRQIKIWFQNRRMKWKKSTVSFNPYEPTGGPWG

SEQ ID NO: 42 - protein, 33 aa, Mus musculus
  MOD_RES 25: PHOSPHORYLATION
DRQIKIWFQNRRMKWKKSTPNFGVYGTSPWGPE

SEQ ID NO: 43 - protein, 38 aa, Mus musculus
  MOD_RES 30: PHOSPHORYLATION
KIKKTKKKGRKKIKKTKKKGRKSTVSFNPYEPTGGPWG

SEQ ID NO: 44 - protein, 38 aa, Mus musculus
  MOD_RES 30: PHOSPHORYLATION
KIKKTKKKGRKKIKKTKKKGRKSTPNFGVYGTSPWGPE

SEQ ID NO: 45 - protein, 33 aa, Homo sapiens
  MOD_RES 25: PHOSPHORYLATION
DRQIKIWFQNRRMKWKKSTVSFNPYEPELAPWA

SEQ ID NO: 46 - protein, 34 aa, Homo sapiens
  MOD_RES 26: PHOSPHORYLATION
DRQIKIWFQNRRMKWKKEAVPLYAWYPNPSTFES